(19) United States
(12) Patent Application Publication
(10) Pub. No.: US 2025/0325704 A1
(43) Pub. Date: Oct. 23, 2025

(54) VIRAL CAPSID PROTEINS WITH SPECIFICITY TO HEART TISSUE CELLS

(71) Applicant: Boehringer Ingelheim International GmbH, Ingelheim am Rhein (DE)

(72) Inventors: Thorsten Lamla, Ingelheim am Rhein (DE); Dragica BLAZEVIC, Ingelheim am Rhein (DE); Stefan MICHELFELDER, Ingelheim am Rhein (DE); Matthias DÜCHS, Ingelheim am Rhein (DE); Sebastian KREUZ, Ingelheim am Rhein (DE); Achim SAUER, Ingelheim am Rhein (DE); Florian MEIER, Ingelheim am Rhein (DE); Birgit STIERSTORFER, Ingelheim am Rhein (DE); Kai Christoph WOLLERT, Hannover (DE); Mortimer KORF-KLINGEBIEL, Duderstadt (DE)

(21) Appl. No.: 18/259,098

(22) PCT Filed: Dec. 23, 2021
(86) PCT No.: PCT/EP2021/087522

§ 371 Date: Jun. 23, 2023

(30) Foreign Application Priority Data
Dec. 23, 2020 (EP) 20217171.6
May 3, 2021 (EP) 21171861.4

(51) Int. Cl. A61K 48/00

(52) U.S. Cl. CPC A61K 48/0058; C12N 2750/14122; C12N 2750/14143

(57) ABSTRACT

This invention generally relates to the field of somatic gene therapy by using viral vectors, and in particular adeno-associated virus (AAV) vectors for the treatment of inherited or acquired diseases. More specifically, the invention relates to a viral capsid protein that provide for a specific transduction of murine endothelial cells for treating or preventing a heart disease in a primate. The viral capsid protein was found to specifically bind to primate heart tissue cells, and in particular primate heart muscle cells, and can be used to provide for an efficient and selective transduction of primate cardiomyocytes and ensure heart tissue-specific expression of one or more transgenes in the primate. The invention further relates to a recombinant viral vector, preferably an AAV vector, which comprises a capsid with at least one transgene packaged in the capsid. The viral vector is suitable for the therapeutic treatment of a cardiac disorder or disease in a primate. The invention further relates to cells and pharmaceutical compositions which comprise the viral vector according to the invention.

### RELATED APPLICATIONS

This application is the U.S. National Stage of International Patent Application No. PCT/EP2021/087522, filed Dec. 23, 2021, which is hereby incorporated by reference in its entirety, and which claims priority to European Patent Application No. 21171861.4, filed May 3, 2021, and European Patent Application No. 20217171.6, filed Dec. 23, 2020.

### SEQUENCE LISTING

The sequences listed in the accompanying Sequence Listing are presented in accordance with 37 C.F.R. 1.822. The Sequence Listing is submitted as an ASCII computer readable text file, entitled SequenceListing_59070.txt, created on Feb. 29, 2024, as 117,377 bytes in size, which is incorporated by reference herein.

### FIELD OF THE INVENTION

This invention generally relates to the field of somatic gene therapy by using viral vectors, and in particular adeno-associated virus (AAV) vectors, for the treatment of inherited or acquired diseases. More specifically, the invention relates to a viral capsid protein that provides for a specific transduction of murine endothelial cells for treating or preventing a heart disease in a primate. The viral capsid protein was found to specifically bind to primate heart tissue cells, and in particular primate heart muscle cells, and can be used to provide for an efficient and selective transduction of primate cardiomyocytes and ensure heart tissue-specific expression of one or more transgenes in the primate. The invention further relates to a recombinant viral vector, preferably an AAV vector, which comprises a capsid with at least one transgene packaged in the capsid. The viral vector is suitable for the therapeutic treatment of a cardiac disorder or disease in a primate. The invention further relates to cells and pharmaceutical compositions which comprise the viral vector according to the invention.

### BACKGROUND OF THE INVENTION

Due to their efficacy and favorable safety profile, vectors based on adeno-associated viruses (AAV) are presently the most widely used vector system for somatic gene therapy. AAV vectors are capable to introduce transgenes as single- or double-stranded DNA into dividing and non-dividing cells of various tissues leading to efficient and long-term stable expression. Clinical trials using recombinant AAV vectors have contributed significantly to the further advancement of gene therapy by achieving important milestones, such as the first market approved AAV-based therapies for the treatment of Leber congenital amaurosis (Luxturna) and spinal muscular atrophy (Zolgensma).

Cardiomyopathies (CMs) are a heterogeneous group of heart muscle diseases and the leading cause for heart failure (HF), the most common cause of morbidity and death in the western world. Upon diagnosis of HF even with the best available treatment, the 5-year survival rate is only about 50% (Writing Group et al, 2016). Current treatment options for CM are mainly symptomatic and cannot halt progression of disease leaving heart transplantation as the only option to prevent HF. For most cardiac illnesses the currently available symptomatic treatment modalities are inadequate. Yet, AAV-based gene therapy has emerged as a promising tool to reverse specific molecular changes for therapeutic intervention in inherited CMs (Chemaly et al, 2013; Tilemann et al, 2012).

Initially, the cardiac specific isoform of the sarcoplasmatic calcium ATPase (SERCA2a) delivered by AAV serotype 1 (AAV1) vector led to the first-in-man study of cardiac AAV gene therapy to treat patients with advanced HF (Jessup et al, 2011; Zsebo et al, 2014). Unfortunately, positive effects seen in a phase 1 clinical trial could not be confirmed in the following phase 2 study; the CUPID2b trial (Greenberg et al, 2016). Retrospective analysis of patient samples showed a very low transduction efficacy and the inability of AAV1 to deliver SERCA2a to cardiomyocytes. In fact, less than 1% of all cardiomyocytes contained virus vector genomes. Therefore, the inability of AAV1 to transduce cells is suggested as the main reason for the negative outcome of the trial.

Several AAV serotypes as well as engineered AAV variants have been tested and compared in preclinical models. Among these, AAV serotype 9 (AAV9) has proven to be most efficient to transduce cardiomyocytes when injected systemically. Consequently, a clinical phase 1 trial for Danon Diseases using AAV9 to express LAMP2B in cardiac tissue (RocketPharma) is currently in the recruiting phase (ClinicalTrials.gov Identifier: NCT03882437). Other variants including AAV6, AAV8 and AAVRh.10 or engineered capsid variants namely AAV-VNS (Ying et al, 2010), M41 (Yang et al, 2009), AAV218 (Asokan et al, 2010) have initially been reported to have superior targeting properties for cardiac gene transfer in mice, but these data could not be transferred into clinically more relevant larger animals yet (Chamberlain et al, 2017) (Tarantal et al, 2017).

Besides AAV9's ability to transduce cardiomyocytes, AAV9 also has a very broad und unspecific tropism to other tissues. This results in 1) a widespread distribution of the vector capsid proteins (including the cargo DNA) to a broad range of tissues and 2) expression of the therapeutic cargo including, but not restricted to liver, the central nervous system, kidney, lung and pancreas. Cardiomyocyte selective expression can be achieved by using cell-type specific promoters, regulatory elements or specific mRNA binding sites introduced in the 3′ prime end of the therapeutic transgene cassette to control gene expression to the intended target tissue (Powell et al, 2015; Qiao et al, 2011). However, most of the available cardiac-specific promoters have a lower activity compared to ubiquitous promoters such as CMV or CAG promoters and finally would require even higher vector doses to achieve sufficient expression levels of a therapeutic cargo (Korbelin et al, 2016a; Korbelin et al, 2016b). In addition to the fact that the packing capacities of AAV limits the use of larger elements, controlling gene expression to a certain tissue or cell-type by using regulatory elements bears always the risk of residual gene expression in off-target tissues caused by a frequently observed leakiness of respective systems. Both, higher vector doses as well as widespread capsid and transgene distribution and therapeutic payload expressed in non-relevant (“off-target”) tissues may cause activation of the immune system (e.g. Tcell activation via TLR9) (Colella et al, 2018), acute decline in platelets, complement activation, or even serious adverse events including acute hepatotoxicity (Wilson & Flotte, 2020).

Several therapeutic proteins have been shown to be useful for ameliorating heart failure and acute myocardial infarction. For example, WO 2014/111458 discloses the use of myeloid-derived growth factor (Mydgf) for treating acute myocardial infarction. Korf-Klingebiel et al. (2015) report that Mydgf is secreted by bone marrow cells after myocardial infarction and promotes cardiomyocyte survival and angiogenesis. Korf-Klingebiel shows that bone marrow-derived monocytes and macrophages produce this protein endogenously to protect and repair the heart after myocardial infarction. Moreover, Korf-Klingebiel shows that treatment with recombinant Mydgf reduces scar size and contractile dysfunction after myocardial infarction. Korf-Klingebiel et al. (2021) described that the transgenic overexpression of Mydgf in bone marrow-derived inflammatory cells attenuated pressure overload-induced hypertrophy and dysfunction. Specifically, the transduction of mice with lentiviral vectors is described. WO 2021/148411 A1 likewise describes the expression of Mydgf in mice that had been transduced with lentivirus. However, lentiviral vectors are associated with severe disadvantages.

Lentiviral vectors offer several attractive properties as gene-delivery vehicles, including: (i) sustained gene delivery through stable vector integration into host genome; (ii) the capability of infecting both dividing and non-dividing cells; (iii) broad tissue tropisms, including important gene- and cell-therapy-target cell types; (iv) no expression of viral proteins after vector transduction; (v) the ability to deliver complex genetic elements, such as polycistronic or intron-containing sequences; (vi) potentially safer integration site profile; and (vii) a relatively easy system for vector manipulation and production (Sakuma et al. 2012). Some of the properties are not always desired. The vector integration into host genome that might be mitigated by potentially safer integration site profile is a liability that should be avoided if possible, especially in connection to the broad tissue tropism that is also not always desired and that may pose a problem if the transgene to be delivered constitutes a risk when expressed outside the target organ. Moreover, while lentiviruses may be a relatively easy system for vector manipulation and production, there is still the need for viral platforms that are easier to handle. Robustness of the virus and the complexity of production beyond lab scale may be factors that have limited the use of lentiviruses so far as well.

Despite the progress that has been made in the field of somatic gene therapy over the last decades, there still is a need for novel viral vectors that allow for the efficient and selective transduction of heart tissue with minimal targeting of other tissues. Such vector should achieve relevant levels of therapeutically active proteins in patients, in particular humans, at a low vector dose, thereby preventing unwanted side effects for the patient. The vectors should also exhibit a low affinity to neutralizing IgGs to allow their use in patients with pre-existing immunity. Therefore, it is an objective of the present invention to provide novel viral vectors that are useful for treating or preventing a heart disease in a primate.

Specifically, these vectors should provide for the efficient and selective transduction of primate heart tissue, and in particular primate cardiomyocytes.

In particular, it is an object of the invention to overcome the shortcomings of lentiviruses. Specifically, it is an object of the invention to provide viral vectors that integrate into the genome less frequently than lentiviruses. Preferably, the viral vectors should only rarely integrate into the genome. Even more preferably, the viral vectors should not at all integrate into the genome at a rate that poses a risk for the patient to be treated, such as an adeno-associated virus (AAV, see Gill-Farina et al, (2016). It is a further object of the invention to provide viral vectors that specifically target the heart and wherein the off-target transduction is lower compared to the AAV known to have heart tropism. Most preferably, both objects are addressed by the viral vectors of the invention. It is a further object of the invention to provide vectors that are easier to manufacture than lentiviruses at a large scale, such as AAVs.

### BRIEF SUMMARY OF THE INVENTION

The present invention relates to a capsid protein which provides for the specific transduction of murine endothelial cells for use in a method of treating or preventing a heart disease in a primate. It has been found that capsid proteins which provide for a selective transduction of murine endothelial cells, and in particular murine endothelial cells of the brain or lung, exert a different tropism in a primate where they provide for a selective transduction of heart tissue cells (in rats the situation was similar to primates). Accordingly, a capsid protein which results in the specific transduction of murine endothelial cells is a highly useful tool for introducing transgenes into heart tissue cells, in particular cardiomyocytes, in a human or non-human primate. The capsid protein can be either unmodified, i.e. naturally occurring, or modified by the insertion of a peptide sequence that influences its tropism. The present invention also provides viral vectors, in particular AAV vectors, with an unmodified or modified capsid that specifically transduce endothelial cells upon administration into a mouse and are hence useful for the selective transduction of cardiomyocytes and transgene expression by systemic vector administration into primates, i.e. human or non-human primates (homo or NHP) in vivo. The vectors lead to a minimum transduction of off-target tissues like CNS, lung, kidney, pancreas and skeletal muscle in the primate. Compared to AAV9, which is the standard vector presently used for cardiac gene therapy and which is studied in an ongoing clinical Phase I trial, the observed liver detargeting of the viral vectors of the present invention resulted in a 43.3-fold lower liver expression, which represents a substantial improvement of the off-target profile. The remarkably improved off-targeting profile could allow for higher dosing, thereby increasing transgene expression and therapeutic efficiency. The viral vectors of the invention are hence particularly suited for the delivery of transgenes to primates suffering from a heart disease, in particular to human patients.

Viral vectors with a modified capsid that provide for a selective homing to and gene expression in a target tissue have been previously described in animal models. Specifically, it has been reported that the incorporation of peptide sequences into the viral capsid proteins is a suitable way of increasing the affinity of the vector system to a certain target tissue. For example, WO 2015/158749 describes an AAV2 variant with a modified capsid protein comprising the peptide NRGTEWD (provided herein as SEQ ID NO:1) that selectively guides the vector to the brain or spinal cord of mice after systemic administration. This AAV2 variant is referred to herein as AAV BI-15.1. Similarly, WO 2015/018860 describes an AAV2 variant with a modified capsid protein comprising the peptide ESGHGYF (provided herein as SEQ ID NO:3) that selectively guides the vector to the lung of mice after systemic administration. This AAV variant is referred to herein as AAV BI-15.2. Both, BI-15.1 and BI-15.2 transduce endothelial cells within their individual target tissues to which they are guided by their specific peptide (BI-15.1 small vasculature of the mouse brain and BI-15.2 pulmonary vasculature of the mouse lung). Such unique vector properties as described for BI-15.1 and BI-15.2 are highly attractive to be applied for the development of targeted gene therapies.

Both vectors, AAV BI-15.1 and AAV BI-15.2, were examined herein for their ability to deliver and express payloads, such as the enhanced green fluorescent protein (eGFP) reporter, in vivo after systemic administration in rats and NHPs. Specifically, AAV BI-15.1 and AAV BI-15.2 were tested in biodistribution studies in two different rat strains (WKY/KyoRj and Sprague Dawley rats) to explore their potency and off-target profile in dose-escalation studies using different promoters and in comparison to AAV9. Most importantly, AAV BI-15.1 and AAV BI-15.2 were analyzed in non-human primates (cynomolgus macaques, NHPs) to investigate the tissue tropism of both vectors in a translationally more relevant species. It was found that the administration of both variants led to the selective transduction of cardiomyocytes both in rats and NHPs. In addition, both variants were also found to selectively transduce iPSC-derived human cardiomyocytes.

While homing to cardiac tissue in rats was nearly at comparable levels for both vectors, AAV BI-15.1 had a more favourable heart to liver homing ratio compared to AAV BI-15.2 in NHPs (0.51 and 0.11). Importantly, both vectors clearly outperformed AAV9 in NHPs with regard to its cardiac to liver homing ratio of approximately 25.7-fold (AAV BI-15.1) and 5,9-fold (AAV BI-15.2), respectively, as compared to reported ratios in the literature (approximately 0.02) (Hordeaux et al, 2018). In addition to the favorable cardiac to liver tissue distribution of vector genomes, AAV BI-15.1 and AAV BI-15.2 also elicited significant transgene expression in cardiomyocytes. This was paralleled by minimal expression in the liver, skeletal muscles and various other tissues. Most importantly, with a dose of 10¹³ vg/kg body weight (BW) AAV BI-15.1 transduced up to 23% of the primate cardiac cells.

Due to the cross-species conservation of the heart-specific tropism in rats and NHPs as well as their efficacy in transducing human iPSCs-derived human cardiomyocytes, it is to be expected that the described tropism of the AAV vectors translates into human cardiomyocytes in vivo. The application of these vectors for the expression of therapeutically relevant molecules (e.g. Mydgf) is therefore useful for establishing effective gene therapy approaches for life-threatening cardiac diseases, where currently only limited treatment options are available.

### BRIEF DESCRIPTION OF THE DRAWINGS

FIG. 1 shows the results of the analysis of purified AAV vector stocks produced for in vivo biodistribution and expression studies using cryo- and negative stain transmission electron microscopy (A) BI-15.1 harbouring a transgene cassette with the enhanced green fluorescence protein (eGFP) under control of the cytomegalovirus early enhancer+chicken β-actin (CAG) promoter and BI-15.2 harbouring eGFP under control of the cytomegalovirus (CMV) promoter were imaged using cryo-transmission electron microscopy (cryoTEM). Representative cryoTEM images show full AAV particles (black arrow), and subpopulation of empty particles (white arrow) or doublets (white arrowhead). Large structures other than AAV (black dashed arrow) and some degree of ice contaminations (white dashed arrow) were observed (upper level A). Packaging statistics were generated by image based internal density analysis (lower level A, B, C) Negative stain transmission electron microscopy (nsTEM) illustrates sample purity and percentage of intact capsids. (B) Size distribution and relative concentrations were automatically detected by nsTEM based images. (C) Four particle classes were defined; primary particles (intact AAV, green), primary broken particles (internally stained, purple) proteasome like particles (small sized impurities, blue) and secondary particles (unknown varying size impurities, red).

FIG. 2 depicts the results obtained from vector distribution and gene expression studies using BI-15.1 and BI-15.2 produced in HEK or Sf9 cells in mice. Three weeks after i.v. administration of; low dose: 5×10¹² vg/kg BW, mid dose: 1×10¹³ vg/kg BW, and high dose: 2×10¹³ vg/kg BW in C57BL/6 mice, vector DNA copies of HEK produced (A) BI-15.1-CAG-eGFP or (B) BI-15.2-CMV-eGFP vector were quantified in homogenized tissue of brain, lung, heart, spleen and liver. Vector copy numbers of BI-15.1 are significantly increased in the brain compared to all other organs of same dose (left panel) ****p<0.0001 (brain vs. liver, spleen, heart and lung in the indicated dose). Vector copy numbers of BI-15.2 are significantly increased in the lung compared to all other organs of same dose ****p<0.0001 (lung vs. liver, spleen, heart and brain or for all doses, respectively **p<0.01 (lung vs. brain in the highest dose). Transgene expression analysis was performed by measurement of eGFP mRNA in the tissues of brain, lung, heart, spleen and liver normalized to the mRNA expression of polymerase II in the sample. (C) BI-15.1-mediated eGFP-mRNA expression in brain and off-target control organs ****p<0.0001 (brain vs. liver, spleen, heart and lung in the indicated dose). (D) BI-15.2 mediated eGFP-mRNA expression in target and off-target control organs ****p<0.0001 (lung vs. liver, spleen, heart and brain in the same dose). Two weeks after i.v. administration of 10¹¹ vg/mouse, vector DNA copies of Sf9 produced (E) BI-15.1-CAG-eGFP or (F) BI-15.2-CMV-eGFP vector were quantified in homogenized tissue of brain, lung and liver.

FIG. 3 shows the results obtained from vector distribution and gene expression studies in rats. The dose-dependent biodistribution and promoter-dependent eGFP expression mediated by BI-15.1 and BI-15.2 vectors in Wistar Kyoto rats are shown. Animals received a low dose (1×10¹³ vg/kg) and a high dose (5×10¹³ vg/kg) of (A) BI-15.1-CAG-eGFP vectors and (B) BI-15.2-CMV-eGFP vectors by intravenous (i.v.) application. Three weeks after AAV application vector DNA copies were quantified in homogenized tissue samples from heart, liver, lung, brain and skeletal muscle (sk.m.) tissue. Analysis of eGFP mRNA levels shows promoter dependent expression in the heart and skeletal muscle mediated by (C) BI-15.1-CAG-eGFP and (D) BI-15.2-CMV-eGFP vectors. Transgene expression analysis was performed by measurement of eGFP mRNA in the tissues of heart, liver, lung, brain and sk.m., normalized to the mRNA expression of polymerase II in the sample. All data are shown as bars (mean)±SD, n=6 animals per group. ****p<0.0001 (vs. liver, lung, brain and sk.m. or as indicated).

FIG. 4 shows the results from studying cardiac expression of eGFP mediated by BI-15.1 and BI-15.2 vectors. (A) Horizontal cuts of whole paraffin-embedded hearts were stained by immunohistochemistry with an eGFP antibody following visualization by DAB staining, three weeks after i.v. application of 1×10¹³ vg/kg (low dose) and 5×10¹³ vg/kg (high dose) of both vectors or of PBS (control) treated Wistar Kyoto rats. Each section represents the heart from one animal of the indicated group. (B) Immunohistochemistry-based area quantification indicates a dose-dependent increase of eGFP expression in cardiomyocytes. For area quantification analysis, data are shown as +SD with plotted data points each representing percentage of eGFP expressing area in heart sections of one individual animal (n=6 animals per vector and n=5 per vehicle treated group).

FIG. 5 shows the results from analyzing vector distribution and gene expression mediated by BI-15.1 and AAV9. Three weeks after i.v. application of 3×10¹³ vg/kg in Sprague Dawley rats, vector DNA copies of (A) BI-15.1-CAG-eGFP or (B) AAV9-CAG-eGFP vector were quantified in homogenized heart, liver, brain, skeletal muscle (sk.m.), lung, kidney, pancreas and spleen tissue samples. Data are shown as bars (mean)+SD, n=8 animals per group. ****p<0.0001 (vs. liver, brain, sk.m., lung kidney pancreas spleen. (C) Analysis of eGFP mRNA levels shows CAG-driven gene expression mediated by BI-15.1 (white bars) and AAV9 (black bars) vectors in various tissues. Data are shown as bars (mean)±SD, n=8 animals per group. P values indicate significant values of BI-15.1 vs. AAV9 as analyzed by student-t test ****p<0.0001; ***p<0.001; **p<0.01; *p<0.1. (D) Efficacy index illustrates the relative expression efficacy of BI-15.1 vs. AAV9 in individual tissues calculated based on their mean RNA expression levels.

FIG. 6 illustrates the heart specific expression pattern of BI-15.1 compared to the widespread expression profile of AAV9 in Wistar Kyoto rats. (A) Three weeks after i.v. application of 3×10¹³ vg/kg of AAV9 (upper panel) and BI-15.1 (lower panel) vectors whole paraffin-embedded tissue sections derived from heart, liver, CNS, lung, kidney and pancreas were stained by immunohistochemistry with an eGFP antibody following visualization by DAB staining. Each section is a representative staining obtained from one section of an animal from the indicated treatment group. (B) Immunohistochemistry-based area quantification of eGFP analyzed in individual tissue sections obtained from PBS injected control group, AAV9 and BI-15.1 injected animals. For area quantification analysis, data are shown as mean values (numbers depicted above the bars)+SD (n=6 animals per vector and n=5 per vehicle treated group). (C) The relative expression efficacy of BI-15.1 vs. AAV9 in individual tissues was calculated based on their mean percentage of transduced area by area quantification and is presented as efficacy index.

FIG. 7 summarizes the data of biodistribution and transgene expression profiles of BI-15.1 vectors in NHPs. Three weeks after intravenous infusion of 1×10¹³ vg/kg of BI-15.1 vectors, vector DNA copy numbers and vector-mediated transgene expression in tissues of cynomolgus macaques were quantified by quantitative real-time PCR (qRT-PCR). (A) Relevant copy numbers of BI-15.1 delivered vector genomes were detected in the atrium (left and right atrium), cardiac ventricle (left and right ventricle), liver and spleen, while other tissues were spared. Data are shown as bars (mean)±SD with plotted individual data points (n=3 animals per group). (B) Predominant BI-15.1 vector mediated eGFP-mRNA expression in the atrium (left and right atrium) as well as the cardiac ventricle (left and right atrium) followed by some expression in the liver. Data are shown as bars (mean)±SD with plotted individual data points (n=3 animals per group). (C) BI-15.1 vector mediated eGFP expression on paraffin-embedded tissue sections of cardiac, liver and brain tissue was stained by immunohistochemistry with an eGFP antibody following visualization by DAB. Each panel (a-o) displays a representative area of the tissue of interest. Each row displays the section of an individually treated animal. (a-c) atrium (d-f) ventricle (g-i) liver (j-o) brain. Size of the scale bars 100 μm. (D) BI-15.1 vector mediated transgene expression in cardiac tissues analyzed by immunohistochemistry-based area quantification and (E) ELISA-based quantitative analysis of eGFP expression in cardiac tissue (right panel). Data are shown as bars (mean)±SD with plotted individual data points (n=3 animals BI 15.1 treated and n=1 PBS treated).

FIG. 8 shows the biodistribution and transgene expression of BI-15.2 vectors in NHPs. Three weeks after intravenous infusion of 1×10¹³ vg/kg of BI-15.2 vectors, vector DNA copy numbers and vector-mediated transgene expression in tissues of cynomolgus macaques were assessed by quantitative real-time PCR (qRT-PCR). (A) Relevant copy numbers of BI-15.2 delivered vector genomes were detected in the liver and spleen followed by the atrium (left and right atrium), cardiac ventricle (left and right ventricle). Some genomic copy numbers was detected in the skeletal muscle (sc. muscle), while other tissues were spared. Data are shown as bars (mean)±SD with plotted individual data points (n=2 animals per group). (B) Predominant BI-15.2 vector mediated eGFP-mRNA expression in the atrium (left and right atrium) as well as the cardiac ventricle (left and right atrium) followed by some expression in the sc. muscle and liver. Data are shown as bars (mean)±SD with plotted individual data points (n=2 animals per group). (C) BI-15.2 vector mediated eGFP expression stained on paraffin-embedded tissue sections of cardiac, liver and brain tissue stained by immunohistochemistry with an eGFP antibody following visualization by DAB. Each panel (a-h) displays a representative area of the tissue of interest. Each row displays the section of an individually treated animal. (a-b) atrium (b-c) ventricle (e-f) liver (g-h) lung. Size of the scale bars 100 μm. (D) BI-15.2 vector mediated transgene expression in cardiac tissues analyzed by immunohistochemistry-based area quantification and (E) ELISA-based quantitative analysis of eGFP expression in cardiac tissue (right panel). Data are shown as bars (mean)±SD with plotted individual data points (n=2 animals BI 15.2 treated and n=1 PBS treated).

FIG. 9 illustrates the ability of AAV9, BI-15.1 and BI-15.2 to transduce and express eGFP in human cardiomyocytes differentiated from induced pluripotent stem cells.

FIG. 10 shows the AAV plasmids maps (pAAV) of (A) pAAV-CAG_huMydgf encoding human Mydgf expression under control of the CAG promoter and (B) pAAVCAG_huMydgf-RTEL encoding the mutant version Mydgf (in which the four Cterminally located amino acids RTEL are deleted) under control of the CAG promoter.

FIG. 11 shows induction of Mydgf and the mutant version Mydgf-RTEL in HEK-293 cells transfected with the sequence of human Mydgf or human Mydgf-RTEL under the control of the CAG-promoter cloned in pAAV expression constructs flanked by Inverted terminal repeats (ITRs; SEQ ID NOs: 16 and 17). 48 hours after transfection, an anti-MYDGF immunoblot was performed using lysed HEK-293 cells or medium. Lanes contain cell lysate (50 μg total protein) or conditioned medium (undiluted).

FIG. 12 shows histology staining for Mydgf in a vertically sliced whole heart of a Sprague Dawley rat three weeks after intravenous infusion of 2.5×10¹¹ vg/kg of BI-15.1-CAG-Mydgf.

FIG. 13 shows histology staining for Mydgf in a vertically sliced whole heart of a Sprague Dawley rat three weeks after intravenous infusion of 2.5×10¹² vg/kg of BI-15.1-CAG-Mydgf. Arrows indicate Mydgf positive stained areas throughout the heart.

FIG. 14 shows histology staining for Mydgf in a vertically sliced whole heart of a Sprague Dawley rat three weeks after intravenous infusion of 2.5×10¹³ vg/kg of BI-15.1-CAG-Mydgf. Arrows indicate Mydgf positive stained areas throughout the heart.

FIG. 15 shows Mydgf mRNA expression measured in heart tissue of Sprague Dawley rats. Three weeks after intravenous infusion of a low (2.5×10¹¹ vg/kg), a mid (2.5×10¹² vg/kg), and a high dose (2.5×10¹³ vg/kg) of BI-15.1-CAG-Mydgf heart tissue was analyzed for Mydgf mRNA expression. A dose dependent increase and measurable levels of Mydgf mRNA were assessed for 2.5×10¹² and 2.5×10¹³ vg/kg. Data are shown as bars (mean)±SD. Each dose was given to one animal, ddCT values were calculated using the housekeeper rat polymerase 2, n.d.=not detectable.

FIG. 16 shows the effects of Mydgf and Mydgf-RTEL protein therapy on left ventricle (LV) remodeling and systolic dysfunction in an ischemia/reperfusion myocardial infarction model. Cardiac function and LV remodeling was assessed by (A) fractional area change (FAC) and (B) LV end-systolic area (LVESA) vs LV end-diastolic area (LVEDA). n=6-8/group, data presented as Mean+/−SEM. ***P<0.001, **P<0.01, *P<0.05 vs. sham; ###P<0.001, ##P<0.01, Mydgf vs. placebo, #P<0.05 Mydgf-RTEL vs. placebo, 1-way ANOVA with Tukey's test.

FIG. 17 shows effects of Mydgf and Mydgf-RTEL protein therapy on angiogenesis and scar size in an ischemia/reperfusion myocardial infarction model. (A) Angiogenesis was assessed by assessing isolectin B4 (IB4)+proliferating endothelial cells in the infarct border zone. ***P<0.001, *P<0.05 vs sham; ##P<0.01 Mydgf vs placebo, ##P<0.01 Mydgf-RTEL vs placebo. (B) Scar size was assessed by analysis of representative tissue sections stained with Masson's trichrome and area is presented as % of left ventricle (LV) size. ##P<0.01 Mydgf WT vs. placebo, #P<0.05 Mydgf truncated vs placebo n=6-8/group, data presented as Mean+/−SEM. 1-way ANOVA with Tukey's test.

### DESCRIPTION OF THE INVENTION

The present invention relates to a capsid protein which provides for a specific transduction of murine endothelial cells for use in a method of treating or preventing a heart disease in a primate, such as a human. The capsid protein of the invention leads to a specific transduction of murine endothelial cells which means that after systemic administration of a viral vector comprising such capsid protein into a mouse, the vector genomes preferably accumulate in endothelial cells, such as endothelial cells of the brain or lung. Accordingly, the number of vector genomes in the endothelial cells of the mouse, such as endothelial cells of the brain or lung, is higher than the number of vector genomes that accumulate in non-endothelial cells. Preferably, the number of vector genomes that can be found in the endothelial cells of the mouse after administration of the vector is 50%, and more preferably 100%, 150%, 200%, 250%, 300%, 350%, 400%, 450%, 500%, 550%, 600%, 650%, 700%, 750%, 800%, 850%, 900%, 1000% or even up to 2000% higher that the number of vector genomes that accumulate in non-endothelial cells. The specificity of transduction can be measured by quantitative PCR methods.

The capsid protein can be an unmodified protein that naturally occurs in a virus. It is however preferred that the capsid protein has been modified to modulate its affinity to a particular target tissue, e.g. by insertion of a peptide sequence which provides for a homing to a target tissue. Suitable peptides which provide for a selective homing to primate heart tissue are provided herein as SEQ ID NO: 1 and SEQ ID NO:2.

Accordingly, it is particularly preferred that the capsid protein used for treating or preventing a heart disease in a primate comprises
(a) the amino acid sequence of SEQ ID NO:1;
(b) the amino acid sequence of SEQ ID NO:2; or
(c) a variant of (a) or (b) which differs from the sequence of SEQ ID NO:1 or SEQ ID NO: 2 by the modification of one amino acid.

In yet another aspect of the invention, a capsid protein is provided for use in a method of treating or preventing a heart disease in a primate, wherein said capsid protein comprises (a) the amino acid sequence of SEQ ID NO:1;
(b) the amino acid sequence of SEQ ID NO:2; or
(c) a variant of (a) or (b) which differs from the sequence of SEQ ID NO:1 or SEQ ID NO: 2 by the modification of one amino acid,
and wherein said capsid protein preferably transduces murine endothelial cells.

The capsid proteins of the invention may comprise a peptide sequence of SEQ ID NO:1 or SEQ ID NO:2. Alternatively, variants of the amino acid sequence of SEQ ID NO:1 or SEQ ID NO:2 can be used which differ from their corresponding reference amino acid sequence by the modification of one amino acid. The modification can be a substitution, deletion or insertion of an amino acid, as long as the variant retains the ability to mediate, as part of the capsid, the specific binding of the vector to the receptor structures of murine endothelial cells and/or primate cardiomyocytes.

The invention encompasses variants of the sequence of SEQ ID NO:1 or SEQ ID NO:2 in which the C- or N-terminal amino acid has been modified. The invention also encompasses variants in which one of the amino acids of SEQ ID NO:1 or SEQ ID NO:2 has been substituted by another amino acid. Preferably, the substitution is a conservative substitution, i.e., a substitution of one amino acid by an amino acid of similar polarity which gives the peptide similar functional properties. Preferably, the substituted amino acid is from the same group of amino acids as the amino acid which is used for the replacement. For example, a hydrophobic residue can be replaced with another hydrophobic residue, or a polar residue by another polar residue. Functionally similar amino acids which can be exchanged for each other by a conservative substitution include, for example, non-polar amino acids such as glycine, valine, alanine, isoleucine, leucine, methionine, proline, phenylalanine, and tryptophan. Examples of uncharged polar amino acids are serine, threonine, glutamine, asparagine, tyrosine, and cysteine. Examples of charged, polar (acidic) amino acids include histidine, arginine and lysine. Examples of charged, polar (basic) amino acids include aspartic acid and glutamic acid. The invention also encompasses variants in which an amino acid has been inserted into the peptide sequence of SEQ ID NO:1 or SEQ ID NO:2. Such insertions can be carried out in any position as long as the resulting variant retains its ability to bind specifically to the receptor structures of murine endothelial cells and/or primate cardiomyocytes. Also encompassed by the invention are variants of the amino acid sequences of the sequence of SEQ ID NO:1 or SEQ ID NO:2 in which a modified amino acid has been introduced. According to the invention, these modified amino acids can be amino acids that have been modified by biotinylation, phosphorylation, glycosylation, acetylation, branching and/or cyclization.

In the below examples, a heptamer sequence comprising the amino acid sequence of SEQ ID NO: 2 and two additional amino acids at the N-terminus, glutamic acid and serine, was used. The heptamer sequence is provided herein as SEQ ID NO:3. However, it could be demonstrated by an alanine scan that the two N-terminal amino acids are not relevant for the specificity of the transduction. As such, only the core structure of SEQ ID NO:2 is responsible for the heart tissue specificity in primates. It should be understood, however, that the heptamer sequence provided herein as SEQ ID NO:3 is merely one embodiment of the amino acid sequence of SEQ ID NO:2 which can be used in the same way as the amino acid sequence of SEQ ID NO:2 for modifying a capsid protein. Hence, in one preferred embodiment, the capsid protein used for treating or preventing a heart disease in a primate comprises the amino acid sequence of SEQ ID NO:3 or a variant thereof which differs from the sequence of SEQ ID NO:3 by the modification of one amino acid.

The present invention therefore provides a capsid protein which is particularly suited for directing therapeutic agents such as viral vectors to heart tissue of a primate. The capsid protein used in a method of the invention has a length of 300 to 800 amino acids, more preferably 400-800 amino acids, and more preferably 500 to 800 amino acids or 600 to 800 amino acids. For example, the capsid protein used in a method of the invention can have a length of at least 100 amino acids, at least 200 amino acids, at least 300 amino acids, at least 400 amino acids, at least 500 amino acids, at least 600 amino acids, or at least 700 amino acids. The

The capsid protein can be derived from any virus which has been used in the field of gene therapy, but it is preferred that the capsid protein used in a method of the invention is one that is derived from a virus belonging to the Parvoviridae family. It is particularly preferred that the capsid protein is derived from an adeno-associated virus (AAV). The AAV can be of any serotype described in the prior art, wherein the capsid protein is preferably derived from an AAV of one of the serotypes 2, 4, 6, 8 and 9. A capsid protein of an AAV of serotype 2 is particularly preferred.

The capsid of the AAV wild-type is made up of the capsid proteins VP1, VP2 and VP3, which are encoded by the overlapping cap gene regions. All three proteins have the same C-terminal region. The capsid of AAV comprises about 60 copies of the proteins VP1, VP2 and VP3, expressed in a ratio of 1:1:8. The peptide sequence of SEQ ID NO:1 or SEQ ID NO:2 or a variant of any of these as defined above can be inserted into any of the capsid proteins VP1, VP2 and VP3, but it is preferred that the peptide sequence is inserted into the capsid protein VP1, more preferably into the capsid protein VP1 of an AAV serotype 2.

In all three capsid proteins of AVV, sites have been identified at which peptide sequences can be inserted to provide for the homing function. Amongst others, the arginine occurring at position 588 (R588) in the VP1 protein of AAV2 has specifically been proposed for the insertion of a homing peptide. This amino acid position of the viral capsid is apparently involved in the binding of AAV2 to its natural receptor. It has been suggested in the prior art that R588 is one of four arginine residues which mediates the binding of AAV2 to its natural receptor. A modification in this region of the capsid is therefore helpful to weaken the natural tropism of AAV2 or to eliminate it completely.

It is therefore preferred according to the present invention that the peptide sequence of SEQ ID NO:1, SEQ ID NO:2, SEQ ID NO:3 or a variant thereof is inserted into the region of amino acids 550-600 of the VP1 protein of AAV2, and more particularly in the region of amino acids 560-600, 570-600, 560-590, or 570-590 of the VP1 protein of AAV2. The wild-type amino acid sequence of the VP1 protein of AAV2 is depicted in SEQ ID NO:4 herein.

It is particularly preferred herein that the peptide sequences are inserted into the peptide with the stuffer sequences exemplified in the below examples. For example, it is preferred that the amino acid sequence of SEQ ID NO:24 is engineered into the capsid protein, such as the VP1 protein of AAV2, in order to provide a capsid protein comprising the amino acid sequence of SEQ ID NO:1. Similarly, it is preferred that the amino acid sequence of SEQ ID NO:25 is engineered into the capsid protein, such as the VP1 protein of AAV2, in order to provide a capsid protein comprising the amino acid sequence of SEQ ID NO:2 or SEQ ID NO:3. As a result of the modification of the protein sequence, the capsid protein of the present invention preferably comprises the amino acid sequence of SEQ ID NO:26 or the amino acid sequence of SEQ ID NO:27.

It is preferred that a sequence is inserted into the amino acid sequence of a viral capsid protein, wherein said sequence comprises or consists of the amino acid sequence of SEQ ID NO: 1, SEQ ID NO:2, SEQ ID NO:3, SEQ ID NO:24, SEQ ID NO:25 or a variant of any of these. Accordingly, in a particularly preferred aspect, the invention relates to a viral capsid protein that comprises the amino acid sequence of SEQ ID NO:1, SEQ ID NO:2, SEQ ID NO:3, SEQ ID NO:24, SEQ ID NO:25, SEQ ID NO:26, SEQ ID NO:27 or a variant of any of these. Table 0 describes how the heptameric sequences NRGTEWD and ESGHGYF can be engineered into a viral capsid such as VP1 of AAV2. After position 588 relative to the wild type AAV2 (SEQ ID NO:4) the heptameric sequences are inserted, flanked by a glycine and an alanine, respectively, which serve as a stuffer. In the AAV2 backbone the asparagine at position 587 is preferably exchanged by glutamine (N587Q).

TABLE 0

| SEQ ID | | Pos. | Pos. | | | | |
|---|---|---|---|---|---|---|---|
| NO: | | 587 | 588 | stuffer | heptamer | stuffer | |
| 4 | QRG | N | R | | — | | QAA |
| 7 | QRG | Q | R | G | NRGTEWD | A | QAA |
| 8 | QRG | Q | R | G | ESGHGYF | A | QAA |
| 24 | | — | | G | NRGTEWD | A | — |
| 25 | | — | | G | ESGHGYF | A | — |

The amino acid sequence of SEQ ID NO:1, SEQ ID NO:2, SEQ ID NO:3, SEQ ID NO:24, SEQ ID NO:25 or a variant of any of these may be inserted behind (i.e. in the direction of the C-terminus) one of the following amino acids of the VP1 protein, in particular of the VP1 protein of SEQ ID NO:4: 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599 or 600. It is particularly preferred that the amino acid sequence of SEQ ID NO:1, SEQ ID NO:2, SEQ ID NO:3, SEQ ID NO:24, SEQ ID NO:25 or a variant of any of these follows amino acid 588 of the VP1 protein of SEQ ID NO:4 (or an respective amino acid position in another capsid protein). In the AAV2 backbone the asparagine at position 587 is preferably exchanged by glutamine (N587Q).

If the peptide sequence of SEQ ID NO:1, SEQ ID NO:2, SEQ ID NO:3, SEQ ID NO:24, SEQ ID NO:25 or a variant of any of these is inserted behind a pre-selected amino acid, e.g. the amino acid in position 588, it might be that one or more amino acids which are the result of the cloning are located between the respective amino acid of the VP1 wild-type and the first amino acid of the homing peptide sequence (stuffer sequence). For example, up to 5 amino acids, i.e. 1, 2, 3, 4 or 5 amino acids, may be located between the respective amino acid of the VP1 wild-type and the first amino acid of the peptide sequence of SEQ ID NO: 1, SEQ ID NO:2, SEQ ID NO:3, SEQ ID NO:24, SEQ ID NO:25 or the variant of any of these.

The sites and regions in the amino acid sequence of the capsid protein indicated above for VP1 apply analogously to the capsid proteins VP2 and VP3 of AAV2. Because the three capsid proteins VP1, VP2 and VP3 of AAV2 differ only by the length of the N-terminal sequence and have an identical C-terminus, a person skilled in the art will have no problem making a sequence comparison to identify the sites indicated above, for the insertion of the peptide ligands, in the amino acid sequences of VP1 and VP2. For example, the amino acid 588 in VP1 corresponds to position R451 of VP2 (SEQ ID NO:5) and/or position R386 of VP3 (SEQ ID NO:6).

Methods for inserting the peptide sequence of SEQ ID NO:1, SEQ ID NO:2, SEQ ID NO: 3, SEQ ID NO:24, SEQ ID NO:25 or variants of any of these into the capsid protein of the viral vector are well known in the field of vector engineering. For example, the nucleic acid sequence encoding the peptide sequence of SEQ ID NO:1, SEQ ID NO:2, SEQ ID NO: 3, SEQ ID NO:24, SEQ ID NO:25 may be cloned into the reading frame of a VP1 gene, such as the gene encoding the AAV2 VP1 protein shown in SEQ ID NO:4. The insertion of the cloned sequence does preferably not lead to any change of the reading frame, nor to a premature termination to translation. The methods required for the above are within the routine skill of a person ordinary skilled in the art working in the field of vector engineering.

In a particularly preferred aspect, the invention provides VP1 proteins of AAV2 which have been modified by the insertion of the peptide sequence of SEQ ID NO:1 or SEQ ID NO: 2 or variants of any of these. For example, SEQ ID NO:7 shows the sequence of the VP1 protein of AAV2 after introduction of the peptide sequence of SEQ ID NO:1. Due to the cloning, the capsid protein has two additional amino acids which do not occur in the native sequence of the VP1 protein of AAV2. Specifically, the peptide sequence of SEQ ID NO: 1 is flanked at its N-terminus by a glycine in position 589, and at its C-terminus by an alanine in position 597. In addition, the asparagine at position 587 of the native sequence is replaced with a glutamine. Similarly, SEQ ID NO:8 shows the sequence of the VP1 protein of AAV2 after introduction of the peptide sequence of SEQ ID NO:3. Due to the cloning, the capsid protein has two additional amino acids which do not occur in the native sequence of the VP1 protein of AAV2. As such, the peptide sequence of SEQ ID NO: 3 is flanked at its N-terminus by a glycine in position 589, and at its C-terminus by an alanine in position 597. In addition, the asparagine at position 587 of the native sequence is replaced with a glutamine.

Therefore, in one embodiment the capsid protein used for treating or preventing a heart disease in a primate comprises
(a) the amino acid sequence of SEQ ID NO:24;
(b) the amino acid sequence of SEQ ID NO:25;
(c) the amino acid sequence of SEQ ID NO:26;
(d) the amino acid sequence of SEQ ID NO:27; or
(e) a variant of (a), (b), (c) or (d) which differs from the sequence of SEQ ID NO:24, SEQ ID NO:25, SEQ ID NO:26 or SEQ ID NO:27 by the modification of one amino acid.

It is particularly preferred that the amino acid sequence of SEQ ID NO:24 or SEQ ID NO: 25, or a variant of any of these, follows the asparagine residue at position 588 (R588) in the VP1 protein of AAV2 (or a respective amino acid position in another capsid protein).

Thus, in a particularly preferred embodiment, the capsid protein for use in the method of the invention is the VP1 protein of AAV2 that has been modified by the insertion of the peptide sequence of SEQ ID NO:1 or a variant thereof. This modified capsid protein comprises the following:
(a) the amino acid sequence of SEQ ID NO:7;
(b) an amino acid sequence having at least 80%, and preferably 90, 95 or 99%, identity to the amino acid sequence of SEQ ID NO: 7 over its entire length; or
(c) a fragment of one of the amino acid sequences defined in (a) or (b).

In another particularly preferred embodiment, the capsid protein for use in the method of the invention is the VP1 protein of AAV2 that has been modified by the insertion of the peptide sequence of SEQ ID NO:2 or a variant thereof. This modified capsid protein comprises the following:
(a) the amino acid sequence of SEQ ID NO:8;
(b) an amino acid sequence having at least 80%, and preferably 90, 95 or 99%, identity to the amino acid sequence of SEQ ID NO: 8 over its entire length; or
(c) a fragment of one of the amino acid sequences defined in (a) or (b).

In another aspect, the invention relates to a viral capsid comprising at least one capsid protein as described herein above for use in a method of treating or preventing a heart disease in a subject in need thereof. In a preferred embodiment, the viral capsid comprises more than one capsid protein as described herein above, such as 2, 3, 4, 5, 6, 7, 8, 9 or 10 capsid proteins. The viral capsid is preferably derived from an AAV, more preferably AAV2.

In another aspect, the invention relates to nucleic acid encoding a capsid protein of any of claims 1-9 for use in a method of treating or preventing a heart disease in a subject in need thereof. The nucleic acid can be DNA or RNA. Preferably, the nucleic acid encoding the capsid protein of the invention is a DNA molecule. Preferably, the nucleic acid is single-stranded DNA (ssDNA) or double-stranded DNA (dsDNA), such as genomic DNA or CDNA.

In another aspect, the invention relates to a plasmid which comprises a nucleic acid as defined above for use in a method of treating or preventing a heart disease in a subject in need thereof. Preferably, the plasmid is a dsDNA molecule that comprises the genome of a complete viral vector.

As used herein, the terms “identical” or “percent identity,” in the context of two or more nucleic acid or polypeptide sequences, refer to two or more sequences or subsequences that are the same in length and/or have a specified percentage of nucleotides or amino acid residues that are the same, when compared and aligned for maximum correspondence.

To determine the percent identity, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical positions/total number of positions (e.g., overlapping positions)×100). In some embodiments, the two sequences that are compared are the same length after gaps are introduced within the sequences, as appropriate (e.g., excluding additional sequence extending beyond the sequences being compared).

The term “% sequence identity to the amino acid sequence of SEQ ID NO: X over the length of SEQ ID NO: X” means that the alignment should cover the entire length of the sequence of SEQ ID NO: X (the reference sequence). In case the algorithms mentioned below do not render an alignment of the entire length of the reference sequence with the test sequence, but only over a subsequence of said reference sequence, amino acid residues within the reference sequence that do not have an identical counterpart on the test sequence are calculated as mismatch. The percent identity score given by said algorithm is then adjusted: If the algorithm yields K identical amino acids over an alignment length of L amino acids, and yields a percent identity of K/L*100, the term L is replaced by the number amino acids of the reference sequence if that number is higher than L. For instance, if the test sequence has one amino acid at the N-terminus less than the reference sequence SEQ ID NO:7 (but is otherwise identical except for this difference), the percent identity is 743/744*100%≈99.8%. The same applies vice versa to nucleic acid sequences.

The determination of percent identity or percent similarity between two sequences can be accomplished using a mathematical algorithm. A preferred, non-limiting example of a mathematical algorithm utilized for the comparison of two sequences is the algorithm of Karlin and Altschul, 1990, Proc. Natl. Acad. Sci. USA 87:2264-2268, modified as in Karlin and Altschul, 1993, Proc. Natl. Acad. Sci. USA 90:5873-5877. Such an algorithm is incorporated into the NBLAST and XBLAST programs of Altschul et al., 1990, J. Mol. Biol. 215:403-410. BLAST nucleotide searches can be performed with the NBLAST program, score=100, wordlength=12, to obtain nucleotide sequences homologous to a nucleic acid encoding a protein of interest. BLAST protein searches can be performed with the XBLAST program, score=50, wordlength=3, to obtain amino acid sequences homologous to a protein of interest. To obtain gapped alignments for comparison purposes, Gapped BLAST can be utilized as described in Altschul et al., 1997, Nucleic Acids Res. 25:3389-3402. Alternatively, PSI-Blast can be used to perform an iterated search which detects distant relationships between molecules (Id.). When utilizing BLAST, Gapped BLAST, and PSI-Blast programs, the default parameters of the respective programs (e.g., XBLAST and NBLAST) can be used. Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis and Robotti, 1994, Comput. Appl. Biosci. 10:3-5; and FASTA described in Pearson and Lipman, 1988, Proc. Natl. Acad. Sci. USA 85:2444-8. Within FASTA, ktup is a control option that sets the sensitivity and speed of the search. If ktup=2, similar regions in the two sequences being compared are found by looking at pairs of aligned residues; if ktup=1, single aligned amino acids are examined. ktup can be set to 2 or 1 for protein sequences, or from 1 to 6 for DNA sequences. The default if ktup is not specified is 2 for proteins and 6 for DNA. Alternatively, protein sequence alignment may be carried out using the CLUSTAL W algorithm, as described by Higgins et al., 1996, Methods Enzymol. 266:383-402.

In another aspect, the invention relates to a recombinant viral vector for use in a method of treating or preventing a heart disease in a subject in need thereof, wherein the vector comprises a capsid and a transgene packaged therein, wherein the capsid comprises at least one capsid protein comprising the amino acid sequence of SEQ ID NO:1 or a variant thereof which differs from the sequence of SEQ ID NO: 1 by the modification of one amino acid. In yet another aspect, the invention relates to a recombinant viral vector for use in a method of treating or preventing a heart disease in a subject in need thereof, wherein the vector comprises a capsid and a transgene packaged therein, wherein the capsid comprises at least one capsid protein comprising the amino acid sequence of SEQ ID NO:2 or a variant thereof which differs from the sequence of SEQ ID NO:2 by the modification of one amino acid. In yet another aspect, the invention relates to a recombinant viral vector for use in a method of treating or preventing a heart disease in a subject in need thereof, wherein the vector comprises a capsid and a transgene packaged therein, wherein the capsid comprises at least one capsid protein comprising the amino acid sequence of SEQ ID NO:3 or a variant thereof which differs from the sequence of SEQ ID NO:3 by the modification of one amino acid. In yet another aspect, the invention relates to a recombinant viral vector for use in a method of treating or preventing a heart disease in a subject in need thereof, wherein the vector comprises a capsid and a transgene packaged therein, wherein the capsid comprises at least one capsid protein comprising the amino acid sequence of SEQ ID NO: 24 or a variant thereof which differs from the sequence of SEQ ID NO:24 by the modification of one amino acid. In yet another aspect, the invention relates to a recombinant viral vector for use in a method of treating or preventing a heart disease in a subject in need thereof, wherein the vector comprises a capsid and a transgene packaged therein, wherein the capsid comprises at least one capsid protein comprising the amino acid sequence of SEQ ID NO:25 or a variant thereof which differs from the sequence of SEQ ID NO: 25 by the modification of one amino acid.

The recombinant viral vector for use in the method of the invention preferably is a recombinant AAV vector. The AAV vector can be derived from an AAV of any serotype, for example, from serotype 2, 4, 6, 8 or 9. It is however most preferred that the recombinant viral vector for use in the method of the invention is derived from AAV serotype 2. The different AAV serotypes differ mainly by their natural tropism. As such, wild-type AAV2 binds more readily to alveolar cells, while AAV5, AAV6 and AAV9 mainly infect epithelial cells. A person skilled in the art can take advantage of these natural differences in the specificity of the cells to further enhance the specificity mediated by the peptides according to the invention for certain cells or tissues. At the nucleic acid level, the various AAV serotypes are highly homologous. For example, serotypes AAV1, AAV2, AAV3 and AAV6 are 82% identical on the nucleic acid level. Liver homing is a general, but unfavorable feature of many, if not most AAV vectors currently tested for cardiac gene therapies. Therefore, a reduction of vector homing to the liver and other tissues is highly desirable. The vectors of the present invention, BI-15.1 and BI-15.2, are associated with a significantly reduced homing to the liver compared to AAV9. To demonstrate this, the number of vector genomes (which corresponds to the number of AAV vector particles) was determined in the heart, the liver and in various other tissues of NHP that had previously been injected with vectors BI-15.1 or BI-15.2. Subsequently, the heart-to-liver or heart-to-tissue ratio was calculated and used as a performance indicator for cardiac homing. The absolute AAV vector genome number in the heart of an NHP was determined by using DNA preparations obtained from tissue lysates of four different histological regions of the heart, namely left atrium, right atrium, left ventricle and right ventricle. The absolute AAV vector genome number in the liver was determined based on DNA preparations obtained from tissue lysates of a section of the median liver lobe. The absolute AAV vector genome number in the kidney was determined based on DNA preparations obtained from tissue lysates of the cortex and the medulla. The absolute AAV vector genome number in the lung was determined based on DNA preparations obtained from tissue lysates of the bronches, the bronchioles and the alveoli. The absolute AAV vector genome number in the brain was determined based on DNA preparations obtained from tissue lysates of the following eight regions where analyzed:

core plexus, brain ventricle, brainstem, cortex, cerebellum, hippocampus, hypothalamus and striatum. The absolute AAV vector genome number in the skeletal muscle was determined based on DNA preparations obtained from tissue lysates of the Musculus gastrocnemius. The absolute AAV vector genome number in the eye was determined based on DNA preparations obtained from tissue lysates of the retina. Quantification of the vector genomes was performed using quantitative polymerase chain reaction (qPCR) using transgene plasmids as a reference standard.

The heart-to-liver or heart-to-tissue ratio in each individual animal was used to calculate the mean heart-to-tissue ratio of BI-15.1 and BI-15.2 (see table below). AAV BI-15.1 had a more favourable heart-to-liver homing ratio in NHPs compared to AAV BI-15.2 (0.515 vs. 0.119). It also had a more favourable heart-to-tissue homing ratio compared to AAV BI-15.2 in various tested tissues. If comparing the calculated heart-to-liver ratios to the corresponding ratios calculated for AAV9 (the heart-to-liver ratio reported for AAV9 in NHPs is approximately 0.02 (Hordeaux et al, 2018)), it can be concluded that BI-15.1 outperforms AAV9 approximately 25.7-fold, and BI-15.2 outperforms AAV9 approximately 5.9-fold.

TABLE 1
mean heart-to-tissue ratios [amount of vector genome copies per 100
ng host DNA homed to the heart divided by vector copy numbers homed
to the indicated tissues per 100 ng host DNA], data are mean values
obtained from tissue sections from n = 3 (BI-15.1) or n = 2 (BI-15.2) NHPs.

| vector | liver | kidney | sk. muscle | lung | brain | spinal cord | ovaries | uterus | eye |
|---|---|---|---|---|---|---|---|---|---|
| BI-15.1 | 0.52 | 52 | 26 | 18 | 105 | 239 | 334 | 24 | 91 |
| BI-15.2 | 0.12 | 12 | 4 | 4 | 15 | 28 | 63 | 25 | 34 |

For therapeutic purposes in primates, preferably in humans, viral vectors having a capsid that comprises at least one capsid protein as defined above, e.g. a capsid protein comprising an amino acid sequence of any of SEQ ID NO:1, 2, 3, 24, 25, 26 or 27, or an amino acid sequence having at least 80% identity to any of these, will be preferably used. It is also preferred that upon administration to an NHP, the viral vectors result in vector genome numbers in the heart of the NHP which are at least 2-fold, at least 2.5-fold, at least 3-fold, at least 3.5-fold, or at least 4-fold higher than in one or more of the following tissues of the same animal: kidney, skeletal muscle, lung, brain, spinal cord, ovary, uterus or eye. In a particular preferred embodiment, the vector genome numbers in the heart of an NHP will be at least 2-fold, at least 2.5-fold, at least 3-fold, at least 3.5-fold, or at least 4-fold higher than in all of the following tissues of the same animal: kidney, skeletal muscle, lung, brain, and spinal cord. It is particularly preferred that the number of vector genomes in the heart of the NHP is a mean value that is determined based on the vector genome numbers determined in the left atrium, right atrium, left ventricle and right ventricle of the heart.

It is similarly preferred that upon administration to a rat, the viral vectors result in vector genome numbers in the heart of the rat which are at least 2-fold, at least 2.5-fold, at least 3-fold, at least 3.5-fold, or at least 4-fold higher than in one or more of the following tissues of the same animal: kidney, skeletal muscle, lung, brain, spinal cord, ovary, uterus or eye. In a particular preferred embodiment, the vector genome numbers in the heart of a rat will be at least 2-fold, at least 2.5-fold, at least 3-fold, at least 3.5-fold, or at least 4-fold higher than in all of the following tissues of the same animal: kidney, skeletal muscle, lung, brain, and spinal cord. It is particularly preferred that the number of vector genomes in the heart of the rat is a mean value that is determined based on the vector genome numbers determined in the left atrium, right atrium, left ventricle and right ventricle of the heart.

In addition, when used for therapeutic purposes in primates, preferably in humans, viral vectors having a capsid that comprises at least one capsid protein as defined above, e.g. a capsid protein comprising an amino acid sequence of any of SEQ ID NO:1, 2, 3, 24, 25, 26 or 27, or an amino acid sequence having at least 80% identity to any of these, preferably transduce heart tissue cells, and in particular cardiomyocytes, of NHPs or rats with a higher specificity than an AAV9 vector. Accordingly, the heart-to-liver ratio will be higher compared to a NHP or rats that received an AAV9 vector. Preferably, the heart-to-liver ratio of a viral vector comprising at least one capsid protein of the invention is at least 2-fold, at least 3-fold, at least 4-fold, at least 5-fold, at least 6-fold, at least 7-fold, at least 8-fold, at least 9-fold, or at least 10-fold higher than the heart to liver ratio of AAV9. It is particularly preferred that the number of vector genomes in the heart of the NHP or rat is a mean value that is determined based on the vector genome numbers determined in the left atrium, right atrium, left ventricle and right ventricle of the heart.

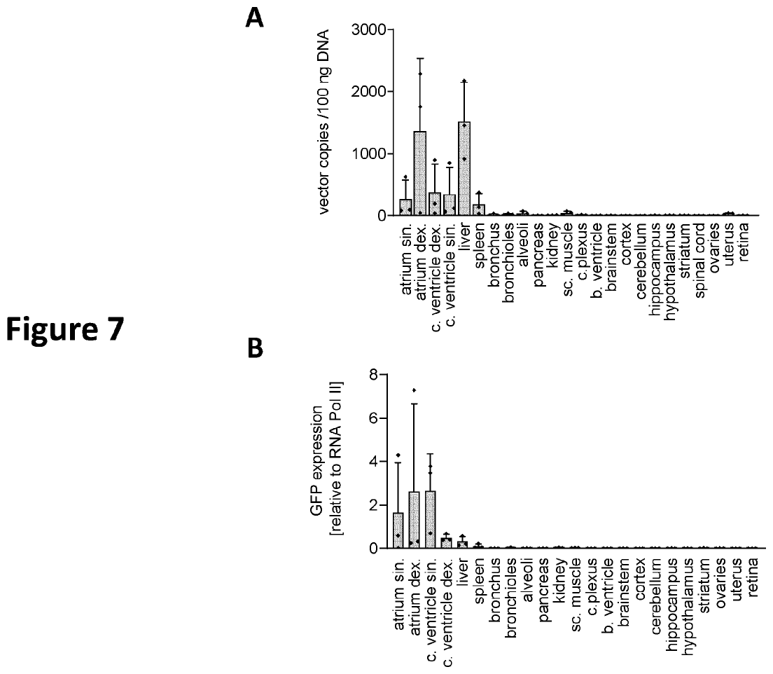

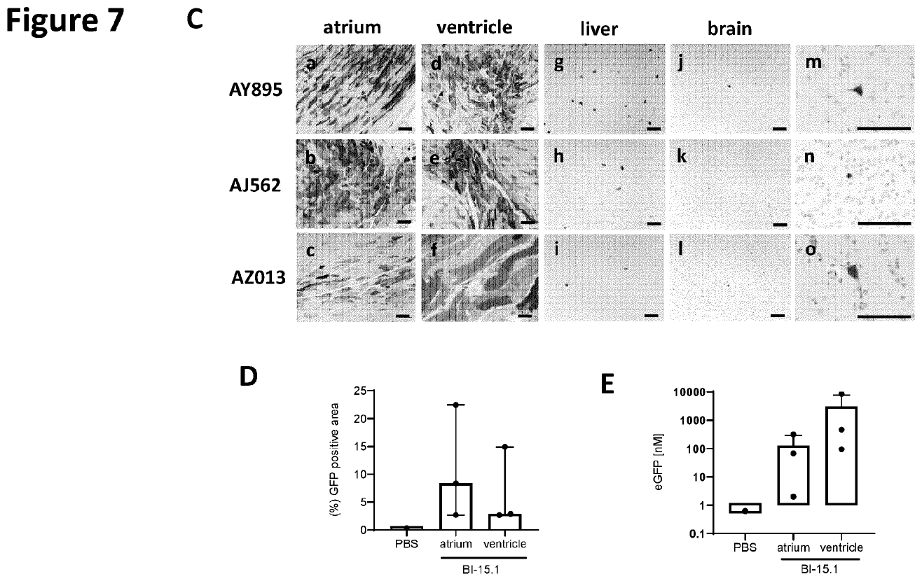

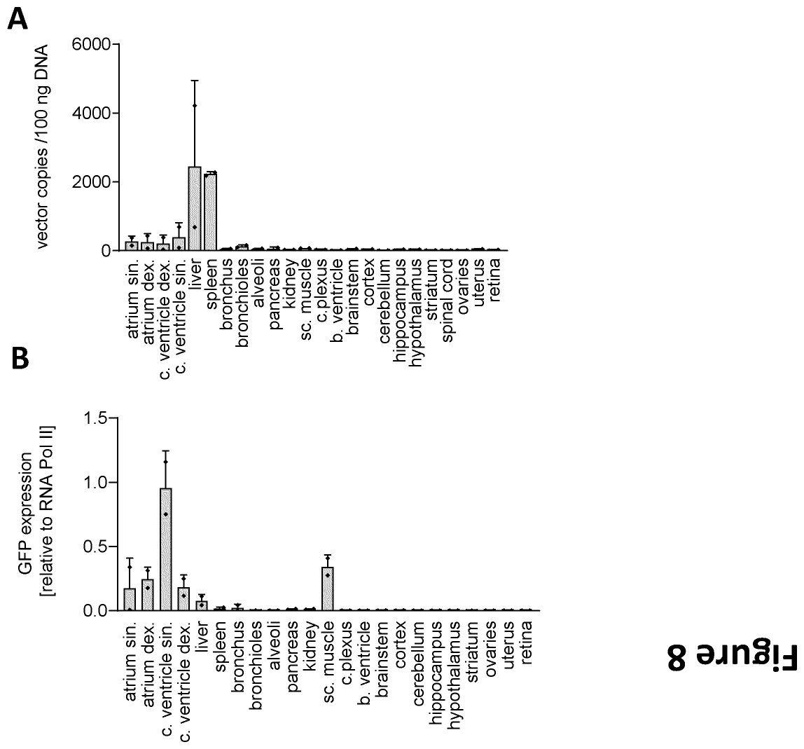

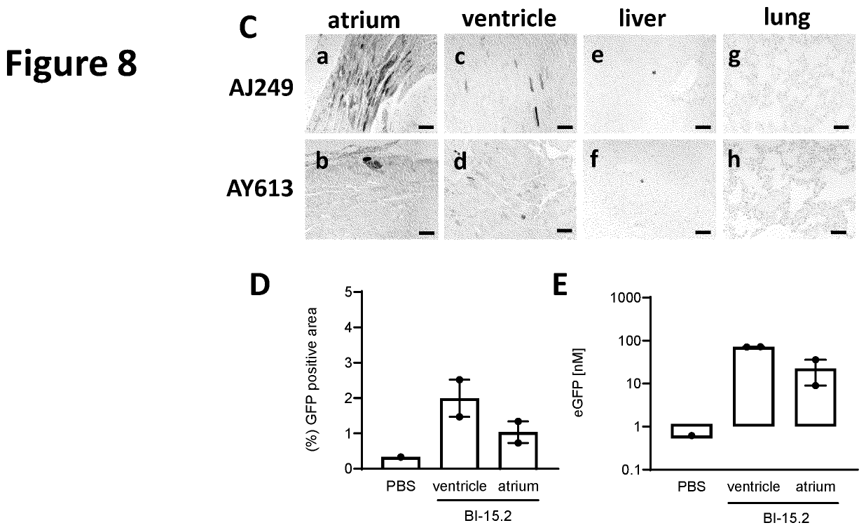

Viral vectors with capsids that comprise at least one capsid protein as defined above specifically transduces murine endothelial cells as well as heart tissue cells, in particular cardiomyocytes, of rats and primates. This means that the viral vectors of the invention have at least about 50%, and more preferably at least about 60%, 70%, 80%, 90%, or 95% of the vector copy heart to liver ratio or GFP heart to liver expression ratio of BI-15.1 or BI-15.2 measured in NHP, such as macaques (as shown in FIGS. 7 and 8).

It is also preferred according to the invention that vectors with capsid proteins comprising a variant of the amino acid sequences shown in SEQ ID NO:1, 2, 3, 24 or 25 have at least about 50%, and more preferably at least about 60%, 70%, 80%, 90%, or 95% of the vector copy heart to liver ratio or GFP heart to liver expression ratio of BI-15.1 or BI-15.2 in NHPs, such as macaques (as shown in FIGS. 7 and 8).

The recombinant viral vector of the present invention comprises a transgene which is packaged therein. As used herein, a transgene refers to a gene that has been introduced by genetic engineering into the genome of the vector and which does not normally belong to the virus genome. The transgene packaged in the recombinant viral vector for use in the method of the invention can be present in the form of a single stranded or double stranded DNA (ssDNA or dsDNA). It can encode any protein that may be helpful in treating or preventing heart disease, such as cardiomyopathy. For example, the transgene can encode a protein which is selected from the group of cardiac repair factors, calcium regulators, or pro-angiogenic factors. In one embodiment, the transgene encodes a cardiac repair factor, such as the human myeloid derived growth factor (huMydgf). It has been reported that this growth factor, which is produced by monocytes and macrophages, promotes heart repair after myocardial infarction (WO 2014/111458, Korf-Klingebiel et al. (2015, 2021), Ebenhoch et al. 2019, WO 2021/148411). The amino acid sequence of huMydgf including the signal sequence is provided herein as SEQ ID NO:18, (without the signal sequence is provided herein as SEQ ID NO 33). The corresponding nucleic acid sequence encoding this protein is provided herein as SEQ ID NO:19. In one embodiment, the transgene packaged in the recombinant viral vector for use in the method of the invention codes for a huMydgf protein:
which comprises or preferably consists of the amino acid sequence of SEQ ID NO: 18 or an amino acid sequence having at least 80%, and preferably at least 90, at least 95, at least 99%, or 100% identity to the amino acid sequence of SEQ ID NO: 18 over its entire length, or
which comprises of the amino acid sequence of SEQ ID NO:33 or an amino acid sequence having at least 80%, and preferably at least 90, at least 95, at least 99%, or 100% identity to the amino acid sequence of SEQ ID NO: 33 over its entire length.

For example, the transgene packaged in the recombinant viral vector for use in the method of the invention may comprise (or consist of) the nucleic acid sequence of SEQ ID NO:19 or a nucleic acid sequence having at least 80%, and preferably at least 90, at least 95, at least 99% or 100% identity to the nucleic acid sequence of SEQ ID NO: 19 over its entire length.

In some embodiments, it may be advantageous to express huMydgf as a mutant protein that lacks the four C-terminal amino acids depicted in SEQ ID NO:18 (RTEL) which represent a putative endoplasmatic reticulum (ER)/Golgi retention signal (Bortnov et al., 2019). ER/Golgi retention signals are known in the art (e.g. Capitani & Sallese (2009)). The amino acid sequence of huMydgf including the signal sequence but lacking the C-terminal amino acids RTEL is provided herein as SEQ ID NO:20 (the amino acid sequence of huMydgf without the signal sequence is provided herein as SEQ ID NO 34). The corresponding nucleic acid sequence encoding this mutant protein is provided herein as SEQ ID NO:21. Therefore, in another embodiment, the transgene packaged in the recombinant viral vector for use in the method of the invention codes for a huMydgf protein
which comprises (or preferably consists) of the amino acid sequence of SEQ ID NO: 20 or an amino acid sequence having at least 80%, and preferably at least 90, at least 95 at least 99%, or 100% identity to the amino acid sequence of SEQ ID NO: 20 over its entire length; or
which comprises of the amino acid sequence of SEQ ID NO:34 or an amino acid sequence having at least 80%, and preferably at least 90, at least 95, at least 99%, or 100% identity to the amino acid sequence of SEQ ID NO: 33 over its entire length and lacks a functional ER/Golgi retention signal.

For example, the transgene packaged in the recombinant viral vector for use in the method of the invention may comprise (or consist of) the nucleic acid sequence of SEQ ID NO:21 or a nucleic acid sequence having at least 80%, and preferably at least 90, at least 95, at least 99%, or 100% identity to the nucleic acid sequence of SEQ ID NO:21 over its entire length.

In another embodiment, the transgene encodes a calcium regulator which is selected from the group consisting of the calcium regulator proteins sarcoplasmic/endoplasmic reticulum Ca2+ ATPase 2a (SERCA2a), small ubiquitin-related modifier 1 (SUMO1) and S100 calcium-binding protein A1 (S100A1). In yet another embodiment, the transgene encodes the pro-angiogenic vascular endothelial growth factor (VEGF).

Targeting heart failure patients with reduced ejection fraction through a gene therapy approach expressing Serca2a has had positive results in a clinical trial (designated as Cupid 1, Efficacy and Safety Study of Genetically Targeted Enzyme Replacement Therapy for Advanced Heart Failure, ClinicalTrials.gov Identifier: NCT00454818). An impaired level of the isoform of sarcoendoplasmic reticulum Ca²⁺ ATPase (SERCA2a) is a typical abnormality in heart failure patients with reduced ejection fraction (HFrEF).

Here, the expression of SERCA2a from an AAV gene cassette, packaged in AAV1, and administered percutaneously resulted in promising data in the restoration of SERCA2a levels and function (Zsebo et al. (2014)). A follow-up CUPID 2-b study (A Study of Genetically Targeted Enzyme Replacement Therapy for Advanced Heart Failure (CUPID-2b, ClinicalTrials.gov Identifier: NCT01643330) unfortunately failed to deliver promising results despite great safety data. It was speculated that the different manufacturing procedures for the CUPID 2-b trial could in part be responsible for the lack of efficacy seen. The DNA levels in the heart muscle measured in patients enrolled in the CUPID 2 trial reported levels of 10 to 192 ssDNA copies per μg of human DNA while preclinical data reported a delivery efficiency of 8 000 to 42 000 copies of viral DNA per μg of host DNA (Lyon et al. (2020)).

Taken together, high doses of AAV1 delivered percutaneously was regarded as safe, but with a significant reduction in DNA delivered to the patient heart compared to the nonclinical studies. Therefore, it is postulated that improving AAV vector cardiactransduction efficiency and selectivity is needed (Bass-Stringer et al. (2018)) and that delivery remains the main challenge (Yamada et al. (2020)) and that improved, higher transduction efficacy, enhanced expression and higher copy number delivery of SERCA2a to the heart muscle should result in clinically beneficial levels of SERCA2a and concomitantly Ca²⁺ levels (Greenberg et al. (2016)).

Similarly, in the CUPID 3 trial started 2021 (Calcium Up-Regulation by Percutaneous Administration of Gene Therapy In Cardiac Disease (CUPID-3), ClinicalTrials.gov Identifier: NCT04703842) the exact construct (AAV1 expressing SERCA2a) is being implemented at a three times higher dose (3×E13) compared to CUPID 2 to improve delivery of the vector to the heart muscle. Alternatively, one can postulate that a novel vector that would enable a more efficient transduction of the human heart muscle at a lower dose combined with improved biodistribution will enable a safer, more efficient drug product. Alternatively, the transgene of the viral vector for use in the method of the invention can encode a microRNA (miRNA). The microRNA preferably is one that is involved in the regulation of the Mitogen-activated protein kinase (MAPK) pathway, the MYOD pathway, the FOXO3 pathway, or the ERK-MAPK pathway. In a particularly preferred embodiment, the microRNA encoded by the transgene of the viral vector for use in the method of the invention is selected from the group consisting of miR-378, miR669a, miR-21 miR212, and miR132.

The transgene of the viral vector for use in the method of the invention can encode a gene that shall supplement a corresponding defective gene in the subject to be treated. Accordingly, the viral vector comprising the transgene is used in a gene therapy approach. Such transgene may encode a protein selected from the group of beta-myosin heavy chain (MYH7), myosin binding protein C (MYBPC3), troponin I (TNNI3), troponin T (TNNT2), tropomyosin alpha-1 chain (TPM1), or myosin light chain (MYL3).

Furthermore, the viral vectors of the invention may also comprise transgenes encoding secretory proteins that are intended for systemic administration into the bloodstream. Such secretory proteins can be efficiently delivered to the bloodstream via the pulmonary capillary bed, which is part of the cardiovascular system.

The transgene can be present in the viral vector in the form of one or more expression cassettes. An expression cassette normally comprises, apart from the transgene, a promotor and a polyadenylation signal. The promotor is operably linked to the transgene. A suitable promoter may be selectively or constitutively active in heart tissue, and in particular in cardiomyocytes. Non-limiting examples of suitable promoters include, but are not limited to, the cytomegalovirus (CMV) promoter or the chicken beta actin/cytomegalovirus hybrid promoter (CAG, SEQ ID NO:14), an endothelial cell-specific promoter such as the VE-cadherin promoter, as well as steroid promoters and metallothionein promoters. In a particularly preferred embodiment, the promoter functionally linked to the transgene is the CAG promoter. In another preferred embodiment, the promoter functionally linked to the transgene is the CMV promoter. In yet another preferred embodiment, the promoter functionally linked to the transgene is a cardiomyocyte-specific promoter. By use of a cardiomyocyte-specific promoter, the specificity of the viral vectors of the invention for heart tissue can be further increased. As used herein, a cardiomyocyte-specific promoter is a promoter whose activity in cardiomyocyte is at least 2-fold, 5-fold, 10-fold, 20-fold, 50-fold or 100-fold higher than in a cell which is not a cardiomyocyte. The expression cassette can also include an enhancer element for increasing the expression levels of exogenous protein to be expressed. Furthermore, the expression cassette can include polyadenylation sequences, such as the SV40 polyadenylation sequences (SEQ ID NO:15) or polyadenylation sequences of bovine growth hormone.

The viral vector of the present invention, such as BI-15.1 or BI-15.2, can be administered to the primate in need of treatment by a number of different ways to which have been extensively described in the prior art. For example, the viral vector can be formulated for various routes of administration, e.g. for intravenous injection or intravenous infusion. The administration can be, for example, by intravenous infusion, for example within 60 minutes, within 30 minutes or within 15 minutes. Alternatively, the viral vector may also be administered locally into the heart, e.g. by intra-myocardial, intra-pericardial, intravascular, trans-vascular administration, or by administration to the area of the left anterior descending artery (LAD) by selective pressure-regulated retro-infusion into the anterior interventricular vein.

Compositions which are suitable for administration by injection or infusion typically include solutions and dispersions, or powders from which solutions and dispersions can be prepared. Such compositions will comprise the viral vector in combination with at least one suitable pharmaceutically acceptable carrier. Suitable pharmaceutically acceptable carriers for intravenous administration include bacterostatic water, Ringer's solution, physiological saline, phosphate buffered saline (PBS) and Cremophor EL™. Sterile compositions for the injection and/or infusion can be prepared by introducing the viral vector in the required amount into an appropriate carrier, and then sterilizing by filtration. Compositions for administration by injection or infusion should remain stable under storage conditions after their preparation over an extended period of time. The compositions can contain a preservative for this purpose. Suitable preservatives include chlorobutanol, phenol, ascorbic acid and thimerosal. The preparation of corresponding formulations and suitable adjuvants is described, for example, in “Remington: The Science and Practice of Pharmacy,” Lippincott Williams & Wilkins; 21st edition (2005). It is preferred herein that the viral vector of the invention is formulated for intravenous administration.

The exact amount of viral vector which must be administered to achieve a therapeutic effect depends on several parameters. Factors that are relevant to the amount of viral vector to be administered include, for example, the route of administration of the viral vector, the nature and severity of the disease, the disease history of the subject being treated, and the age, weight, height, and health of the subject to be treated. Furthermore, the expression level of the transgene which is required to achieve a therapeutic effect, the immune response of the patient, as well as the stability of the gene product are relevant for the amount to be administered. A therapeutically effective amount of the viral vector can be determined by a person skilled in the art on the basis of general knowledge and the present disclosure. The viral vector is preferably administered in an amount corresponding to a dose of virus in the range of 1.0×10⁸ to 1.0×10¹⁵ vg/kg (virus genomes per kg body weight), although a range of 1.0×10¹⁰ to 1.0×10¹⁵ vg/kg, 1.0×10¹² to 5.0×10¹⁴ vg/kg or 1.0×10¹¹ to 1.0×10¹³ vg/kg is more preferred. The amount of the viral vector to be administered, such as the AAV2 vector according to the invention, for example, can be adjusted according to the strength of the expression of one or more transgenes.

In another aspect, the invention relates to a method for producing a viral vector in which a plasmid is used which encodes a capsid protein comprising the amino acid sequence of SEQ ID NO:1, SEQ ID NO:2, SEQ ID NO:3, SEQ ID NO:24, SEQ ID NO:25 or a variant of any of these. For example, the viral vector can comprise the amino acid sequence of SEQ ID NO:7 or SEQ ID NO:8. The viral vectors of the present invention can be prepared in accordance with well-known methods described in the art. For example, the basic method of producing recombinant AAV vectors comprising a transgene is described in detail in the prior art (Xiao et al, 1998). For example, HEK 293-T cells are transfected with three plasmids. A first plasmid comprises the cap and rep regions of the AAV genome, but the naturally occurring inverted repeats (ITRs) are missing. A second plasmid comprises a transgene expression cassette which is flanked by the corresponding ITRs, which constitute the packaging signal. The expression cassette is therefore packaged into the capsid in the course of the assembly of the viral particles. The third plasmid is an adenoviral helper plasmid which encodes the helper proteins E1A, E1, E2A, E4-orf6, VA, which are required for AAV replication in the HEK 293-T cells. Alternatively, it is also possible to produce the AAV vectors in insect cells. Suitable methods for producing the viral vectors in Sf9 insect cells are described, for example, in WO 2015/158749 or US20170029464A1. The purity of the viral vectors can be checked by suitable methods such as PCR amplification. The viral vectors of the invention can be purified, for example, by gel filtration, or by caesium chloride or iodixanol gradient ultracentrifugation. The viral vectors used for administration should be substantially free of wild-type and replication-competent virus.

In another aspect, the invention relates to a cell that comprises capsid protein, a nucleic acid encoding same, a plasmid comprising such a nucleic acid, or a recombinant viral vector as described above, for use in a method of treating or preventing a heart disorder or disease. The cell preferably is a human cell or cell line. In one embodiment, a cell has been obtained, for example, from a human subject by biopsy and then transfected with the viral vector in an ex vivo procedure. The cell can then be re-implanted or supplied in other ways to the subject in other ways, e.g. by transplantation or infusion. The likelihood of rejection of transplanted cells is lower when the subject from which the cell was derived is genetically similar to the subject to which the cell is administered. It is therefore preferred, that the subject to whom the transfected cells are supplied is the same subject from which the cells were previously obtained. The cell preferably is a human heart tissue sale, in particular a human cardiomyocyte. The cell to be transfected can also be a stem cell, such as a human adult stem cell. It is particularly preferred according to the invention that the cells to be transfected are autologous cells that have been transfected ex vivo with the viral vector according to the invention, for example the recombinant AAV2 vector described above.

In another aspect, the invention relates to a pharmaceutical composition comprising a capsid protein, a nucleic acid, a plasmid, or a recombinant viral vector as defined above for use in a method of treating or preventing a heart disease in a primate. The heart disease to be treated preferably is a cardiomyopathy.

The invention relates to the use of a capsid protein, nucleic acid, plasmid, recombinant viral vector, or pharmaceutical composition as defined above for treating or preventing a heart disease in a primate. The heart disease to be treated preferably is a cardiomyopathy. The cardiomyopathy is preferably selected from the group consisting of hypertrophic cardiomyopathy (HCM), dilated cardiomyopathy (DCM), arrythmogenic right ventricular cardiomyopathy (ARVC), restrictive cardiomyopathy (RCM) and left ventricular noncompaction cardiomyopathy (LVNC). Table 1 provides an overview of the therapeutically active proteins that are useful in the treatment of a cardiomyopathy in humans.

The subject to be treated is a human or non-human primate. Non-human primates include, but not limited to, monkeys, squirrel monkeys, owl monkeys, baboons, chimpanzees, marmosets, gorillas, apes, lemurs, macaques and gibbons. In a preferred embodiment, the non-human primate is a chimpanzee. Further, as used herein a human primate comprises a human.

In another aspect, the invention relates to the use of a capsid protein, nucleic acid, plasmid, recombinant viral vector, or pharmaceutical composition as defined above for the manufacture of a medicament for treating or preventing a heart disease in a primate. The heart disease to be treated preferably is a cardiomyopathy.

TABLE 1
Non-comprehensive list of targets for cardiomyopathies

| | Type of | Suggested mode of | | |
|---|---|---|---|---|
| Target | molecule | action/effect | Therapeutic approach | Reference |
| Myeloid-derived Growth | protein | pro-angiogenic/proliferative | supplementation | Korf-Klingenbeil et al. 2016 |
| Factor (myDGF) | | | | |
| Sarcoplasmic/endoplasmic | protein | calcium regulation | supplementation | Chamberlain et al. 2017 |
| reticulum calcium ATPase | | | | |
| 2a (SERCA2a) | | | | |
| Small ubiquitin-related | protein | calcium regulation | supplementation | Chamberlain et al. 2017 |
| modifier 1 (SUM01) | | | | |
| S100 calcium-binding | protein | calcium regulation | supplementation | Chamberlain et al. 2017 |
| protein A1 (S100A1) | | | | |
| Vascular Endothelial | protein | pro-angiogenic | supplementation | Chamberlain et al. 2017 |
| Growth Factor (VEGF) | | | | |
| miR-378 | microRNA | pro-apoptotic | inhibition | Xu et al. 2016 |
| miR669a | microRNA | pro-regenerative | overexpression | Pang et al. 2019 |
| miR-21 | microRNA | profibrotic | inhibition | Xu et al. 2016 |
| miR212 | microRNA | heart remodeling | inhibition | Ucar et al. 2012 |
| miR132 | microRNA | cardioprotective | overexpression | Foinqiunos et al. 2020 |
| Beta-myosin heavy chain | DNA | structural muscle protein | supplementation | Chamberlain et al. 2017 |
| (MYH7) | | | | |
| Myosin binding protein C | DNA | structural muscle protein | supplementation | Chamberlain et al. 2017 |
| (MYBPC3) | | | | |
| Troponin I (TNNI3) | DNA | structural muscle protein | supplementation | Chamberlain et al. 2017 |
| Troponin T (TNNT2) | DNA | structural muscle protein | supplementation | Chamberlain et al. 2017 |
| Tropomyosin alpha-1 | DNA | structural muscle protein | supplementation | England et al. 2017 |
| chain (TPM1) | | | | |
| Myosin light chain (MYL3) | DNA | structural muscle protein | supplementation | Chamberlain et al. 2017 |

In a further aspect, the invention relates to a method of treating or preventing a heart disease in a primate, said method comprising the administration of a viral vector according to the invention, preferably an AAV vector as described above, to a primate, such as a human or non-human primate. The vector preferably comprises a capsid which has at least one capsid protein containing the amino acid sequence of SEQ ID NO:1 or SEQ ID NO:2 or a variant of any of these. In a particularly preferred embodiment, the vector comprises a capsid which has at least one capsid protein comprising or consisting of the amino acid sequence of SEQ ID NO:7 or SEQ ID NO:8 or fragment of any of these. The viral vector preferably further comprises a transgene, for example a gene encoding a therapeutic protein, which is useful for treating or preventing a heart disease. After administration to the primate, the vector provides for specific expression of the transgene in heart tissue cells of the primate.

Further embodiments of the invention are described hereinafter.

In one embodiment of the invention, the invention relates to a capsid protein which provides for the specific transduction of murine endothelial cells for use in a method of treating or preventing a heart disease in a primate, wherein said method of treating or preventing a heart disease comprises the transduction of primate cardiomyocytes. WO 2019/199867 refers to the use of AAV 15.1 but not in the context of the transduction of primate cardiomyocytes. The target organ for gene therapy mediated by the viral vectors described in WO 2019/199867 is obviously not the heart.

In a second embodiment, the invention relates to a capsid protein for use in a method of treating or preventing a heart disease in a primate, wherein said method of treating or preventing a heart disease comprises the transduction of primate cardiomyocytes, said capsid protein comprising:
(a) the amino acid sequence of SEQ ID NO:1;
(b) the amino acid sequence of SEQ ID NO:2 or 3; or
(c) a variant of (a) or (b) which differs from the sequence of SEQ ID NO:1, SEQ ID NO:2 or SEQ ID NO:3 by the modification of one amino acid.

A fourth embodiment of the invention relates to a capsid protein for use in a method of treating or preventing cardiomyopathy, said capsid protein comprising:
(a) the amino acid sequence of SEQ ID NO:1;
(b) the amino acid sequence of SEQ ID NO:2 or 3; or
(c) a variant of (a) or (b) which differs from the sequence of SEQ ID NO:1, SEQ ID NO:2 or SEQ ID NO:3 by the modification of one amino acid.

For instance, the capsid protein may be a part of a viral vector that delivers huMydgf to the heart and more specifically transduces cardiomyocytes. The use of huMydgf for the treatment of cariomypathy is known (WO 2014/111458, Korf-Klingebiel et al. (2015, 2021), Ebenhoch et al. 2019, WO 2021/148411).

A fifth embodiment of the invention relates to a capsid protein for use in a method of any of the aforementioned embodiments, wherein said capsid protein has a length of 300 to 800 amino acids.

A sixth embodiment of the invention relates to a capsid protein for use in a method of any of the aforementioned embodiments, wherein said capsid protein is a capsid protein of a virus belonging to the Parvoviridae family, and preferably a capsid protein of an adeno-associated virus (AAV).

A seventh embodiment of the invention relates to a capsid protein for use in a method of any of the aforementioned embodiments, wherein said AAV is selected from the group consisting of AAV serotype 2, 4, 6, 8 and 9, and wherein said AAV is preferably serotype 2.

An eighth embodiment of the invention relates to a capsid protein for use in a method of claim 7, wherein said capsid protein is a VP1 protein of an AAV serotype 2.

A ninth embodiment of the invention relates to a capsid protein for use in a method of any of the aforementioned embodiments, wherein said amino acid sequence of SEQ ID NO:1, SEQ ID NO:2 or SEQ ID NO:3 or said variant thereof is inserted in the region of amino acids 550-600 of the capsid protein.

Further embodiments relate to a
(i) capsid protein for use in a method of any of the aforementioned embodiments, wherein said capsid protein comprises:
   (a) the amino acid sequence of SEQ ID NO: 7 or SEQ ID NO: 8;
   (b) an amino acid sequence having at least 85%, at least 90%, at least 95%, at least 98%, at least 99%, or 100% identity to the amino acid sequence of SEQ ID NO: 7 or SEQ ID NO: 8; or
   (c) a fragment of one of the amino acid sequences defined in (a) or (b).
(ii) capsid protein for use in a method of any of the aforementioned embodiments, wherein said capsid protein comprises:
   (a) the amino acid sequence of SEQ ID NO:24;
   (b) the amino acid sequence of SEQ ID NO:25;
   (c) the amino acid sequence of SEQ ID NO:26;
   (d) the amino acid sequence of SEQ ID NO:27; or
   (e) a variant of (a), (b), (c) or (d) which differs from the sequence of SEQ ID NO: 24, SEQ ID NO:25, SEQ ID NO:26 or SEQ ID NO:27, respectively, by the modification of one amino acid.
(iii) viral capsid comprising a capsid protein of any of the aforementioned embodiments for use in a method of treating or preventing a heart disease in a primate, wherein said method of treating or preventing a heart disease comprises the transduction of primate cardiomyocytes.
(iv) nucleic acid encoding a capsid protein of any of the aforementioned embodiments for use in a method of treating or preventing a heart disease in a primate, wherein said method of treating or preventing a heart disease comprises the transduction of primate cardiomyocytes.
(v) plasmid which comprises a nucleic acid according to (iv) for use in a method of treating or preventing a heart disease in a primate, wherein said method of treating or preventing a heart disease comprises the transduction of primate cardiomyocytes.

In a further embodiment of the invention, the invention relates to a recombinant viral vector, wherein the vector comprises a capsid and a transgene packaged therein,
wherein the capsid comprises at least one capsid protein comprising
(a) the amino acid sequence of SEQ ID NO:1;
(b) the amino acid sequence of SEQ ID NO:2;
(c) the amino acid sequence of SEQ ID NO:3;
(d) a variant of (a), (b) or (c) which differs from the sequence of SEQ ID NO:1, SEQ ID NO:2, or SEQ ID NO:3 by the modification of one amino acid;
(e) the amino acid sequence of SEQ ID NO:24;
(f) the amino acid sequence of SEQ ID NO:25;
(g) the amino acid sequence of SEQ ID NO:26;
(h) the amino acid sequence of SEQ ID NO:27;
(i) a variant of (e), (f), (g) or (h) which differs from the sequence of SEQ ID NO: 24, SEQ ID NO:25, SEQ ID NO:26, SEQ ID NO:27 by the modification of one amino acid,
(j) the amino acid sequence of SEQ ID NO:7 or SEQ ID NO:8; or
(k) an amino acid sequence having at least 80, 85, 90, 95, 98, 99, or 100% identity to the amino acid sequence of SEQ ID NO:7 or SEQ ID NO:8;
for use in any of the following methods (A), or (B) or (C):
(A) in a method of treating or preventing a heart disease in a primate, wherein said method of treating or preventing a heart disease comprises the transduction of primate cardiomyocytes; or
(B) in a method of treating or preventing cardiomyopathy in a primate; or
(C) in a method of treating or preventing heart failure or chronic heart failure in a primate.

For instance, the viral vector may delivers huMydgf or SERCA2a to the heart and more specifically transduces cardiomyocytes. The use of huMydgf for the treatment of cardiomypathy and heart failure is known for Mydgf. The use of SERCA2a for the treatment of (chronic) heart failure or heart failure patients with reduced ejection fraction is known, too.

Further embodiments relate to a
(i) Recombinant viral vector for use in the preceding embodiment, wherein said cardiomyopathy is selected from the group consisting of hypertrophic cardiomyopathy (HCM), dilated cardiomyopathy (DCM), arrythmogenic right ventricular cardiomyopathy (ARVC), restrictive cardiomyopathy (RCM) and left ventricular noncompaction cardiomyopathy (LVNC).
(ii) Recombinant viral vector for use in for use in the preceeding embodiment, wherein said cardiomyopathy is selected from the group consisting of primary cardiomyopathy, preferably inherited cardiomyopathy, cardiomyopathy caused by spontaneous mutations, and acquired cardiomyopathy, preferably ischemic cardiomyopathy caused by atherosclerotic or other coronary artery diseases, cardiomyopathy caused by infection or intoxication of the myocardium.
(iii) Recombinant viral vector for use in for use in the preceeding embodiment, wherein said heart disease is selected from the group consisting of angina pectoris, cardiac fibrosis and cardiac hypertrophy.
(iv) Recombinant viral vector for use in for use in the preceeding embodiment, wherein said heart failure or chronic heart failure is heart failure with preserved ejection fraction (HFpEF), heart failure with reduced ejection fraction (HFrEF), or heart failure with mid-range ejection fraction (HFmrEF).
(v) Recombinant viral vector for use in for use in the preceeding embodiment, wherein said HFpEF is stage C or stage D HFpEF or wherein said HFrEF is stage C or stage D HFrEF.

For instance, the use of huMydgf for the treatment of as mentioned above is known and described in WO 2014/111458, Korf-Klingebiel et al. (2015, 2021), Ebenhoch et al. 2019, WO 2021/148411.

Further embodiments relate to a recombinant viral vector for use in for use in a preceding embodiment, wherein said vector is a recombinant AAV vector
(i) is selected from the group consisting of AAV serotype 2, 4, 6, 8 and 9, and preferably AAV serotype 2;
(ii) wherein the transgene is in the form of an ssDNA or a dsDNA;
(iii) wherein the transgene encodes a protein selected from the group of cardiac repair factors, calcium regulators, or pro-angiogenic factors;
(iv) wherein the transgene encodes the cardiac repair factor huMydgf.
(v) wherein the transgene encodes a calcium regulator selected from the group consisting of SERCA2a, SUMO1 and S100A1;
(vi) wherein the transgene encodes the pro-angiogenic factor VEGF;
(vii) wherein the transgene encodes a microRNA (miRNA), preferably wherein said microRNA is involved in the regulation of the MAPK pathway, the MYOD pathway, the FOXO3 pathway, or the ERK-MAPK pathway, more preferably wherein said microRNA is selected from the group consisting of miR-378, miR669a, miR-21 miR212, and miR132;
(viii) wherein the transgene is a gene that shall supplement a defective gene in the primate to be treated, preferably wherein said gene encodes a protein selected from the group of beta-myosin heavy chain (MYH7), myosin binding protein C (MYBPC3), troponin I (TNNI3), troponin T (TNNT2), tropomyosin alpha-1 chain (TPM1), or myosin light chain (MYL3), wherein the vector is formulated for intravenous administration.

In a further embodiment of the invention, the invention relates to a recombinant viral vector, comprising a capsid and a transgene packaged therein, wherein the capsid comprises at least one capsid protein comprising
(a) the amino acid sequence of SEQ ID NO:1;
(b) the amino acid sequence of SEQ ID NO:2;
(c) the amino acid sequence of SEQ ID NO:3; or
(d) a variant of (a) (b) or (c) which differs from the sequence of SEQ ID NO:1, SEQ ID NO:2 or SEQ ID NO:3 by the modification of one amino acid;
and wherein the transgene encodes a protein comprising.
(a) the amino acid sequence of SEQ ID NO: 18, 20, 33 or 34;
(b) an amino acid sequence having at least 80%, at least 85%, at least 90%, at least 95%, at least 98% or at least 99%, or 100% identity to the amino acid sequence of SEQ ID NO: 18, 20, 33 or 34; or
(c) a fragment of one of the amino acid sequences defined in (a) or (b) that has the function of huMydgf and has preferably a length of more than 100, more preferably 120 amino acids.

Preferably, the protein variant or fragment preserves at least part of the activity, and more preferably the complete activity of huMydgf, as determined by the assay according to Material and methods (i) according to 1.12 in conjunction with 1.14 and 1.15. The activity is deemed to be preserved if the the protein variant or fragment shows relevant biological effects in 1.12 and 1.14 and 1.15. Furthermore, preferably the protein variant or fragment has the potency of huMydgf in the activity assay of 1.16. For comparison purposes, it is prefered to use huMydgf having a sequence according to SEQ ID NO: 35.

Further embodiments relate to a recombinant viral vector according to one or more preceding embodiments, said capsid protein comprises:
(a) the amino acid sequence of SEQ ID NO:24;
(b) the amino acid sequence of SEQ ID NO:25;
(c) the amino acid sequence of SEQ ID NO:26;
(d) the amino acid sequence of SEQ ID NO:27;
(e) a variant of (a), (b), (c) or (d) which differs from the sequence of SEQ ID NO: 24, SEQ ID NO:25, SEQ ID NO:26, or SEQ ID NO:27 by the modification of one amino acid.

Further embodiments relate to a recombinant viral vector according to one or more preceding embodiments, comprising a capsid and a transgene packaged therein, wherein the capsid comprises at least one capsid protein comprising
(a) the amino acid sequence of SEQ ID NO:1;
(b) the amino acid sequence of SEQ ID NO:2;
(c) the amino acid sequence of SEQ ID NO:3; or
(d) a variant of (a) (b) or (c) which differs from the sequence of SEQ ID NO:1, SEQ ID NO:2 or SEQ ID NO:3 by the modification of one amino acid;
wherein the transgene encodes a protein that lacks a functional Golgi/endoplasmatic reticulum retention signal, preferably a protein that comprises the sequence of SEQ ID NO:20 or SEQ ID NO:34.

Further embodiments relate to a recombinant viral vector wherein the transgene encodes a protein comprising:
(a) the amino acid sequence of SEQ ID NO: 18, 20, 33 or 34;
(b) an amino acid sequence having at least 80%, at least 85%, at least 90%, at least 95%, at least 98% or at least 99%, or 100% identity to the amino acid sequence of SEQ ID NO: 18, 20, 33 or 34; or
(c) a fragment of one of the amino acid sequences defined in (a) or (b) that has the function of huMydgf and has preferably a length of more than 100, more preferably 120 amino acids,
for use in a method of treating or preventing
(i) a heart disease in a primate, wherein said method of treating or preventing a heart disease comprises the transduction of primate cardiomyocytes; or
(ii) cardiomyopathy in a primate; or
(iii) heart failure or chronic heart failure in a primate.

Preferably, the protein variant or fragment preserves at least part of the activity, and more preferably the complete activity of huMydgf, as determined by the assay according to Material and methods (i) according to 1.12 in conjunction with 1.14 and 1.15. The activity is deemed to be preserved if the the protein variant or fragment shows relevant biological effects in 1.12 and 1.14 and 1.15. Furthermore, preferably the protein variant or fragment has the potency of huMydgf in the activity assay of 1.16. For comparison purposes, it is prefered to use huMydgf having a sequence according to SEQ ID NO: 35.

Further embodiments relate to a recombinant viral vector according to one or more preceding embodiments, comprising a capsid and a transgene packaged therein, wherein the capsid comprises at least one capsid protein comprising
(a) the amino acid sequence of SEQ ID NO:1;
(b) the amino acid sequence of SEQ ID NO:2;
(c) the amino acid sequence of SEQ ID NO:3; or
(d) a variant of (a) (b) or (c) which differs from the sequence of SEQ ID NO:1, SEQ ID NO:2 or SEQ ID NO:3 by the modification of one amino acid;
and wherein the transgene encodes a human calcium regulator SERCA2a, wherein the human calcium regulator SERCA2a preferably comprises
(a) an amino acid sequence of any of SEQ ID NOs: 28-32;
(b) an amino acid sequence having at least at least 80%, at least 85%, at least 90%, at least 95%, at least 98% or at least 99%, or 100% identity to one of the amino acid sequences of SEQ ID NOs: 28-32; or
(c) a fragment of one of the amino acid sequences defined in (a) or (b), and
wherein said capsid protein preferably comprises:
(a) the amino acid sequence of SEQ ID NO:24;
(b) the amino acid sequence of SEQ ID NO:25;
(c) the amino acid sequence of SEQ ID NO:26;
(d) the amino acid sequence of SEQ ID NO:27;
(e) a variant of (a), (b), (c) or (d) which differs from the sequence of SEQ ID NO: 24, SEQ ID NO:25, SEQ ID NO:26, or SEQ ID NO:27 by the modification of one amino acid.

A further embodiment of the invention relates to a recombinant viral vector wherein the transgene encodes a human calcium regulator SERCA2a, for use in a method of treating or preventing
(i) a heart disease in a primate, wherein said method of treating or preventing a heart disease comprises the transduction of primate cardiomyocytes;
(ii) chronic heart failure; or
(iii) heart failure patients with reduced ejection fraction.

Further embodiments relates to a pharmaceutical composition comprising a capsid protein of according to the invention, a viral capsid according to the invention, a nucleic acid according to the invention, a plasmid according to the invention, or a recombinant viral vector according to the invention for medical use, preferably for a method of treating or preventing a heart disease of a primate according to the invention. The method of treatment or prevention is directed preferably to a primate that is a human.

Further embodiments relates to the use of a pharmaceutical composition comprising a capsid protein of according to the invention, a viral capsid according to the invention, a nucleic acid according to the invention, a plasmid according to the invention, or a recombinant viral vector according to the invention for the manufacture of a medicament for treating or preventing a heart disease in a primate, preferably a human.

Further embodiments relates to a method of treating a primate the use of a pharmaceutical composition comprising a capsid protein of according to the invention, a viral capsid according to the invention, a nucleic acid according to the invention, a plasmid according to the invention, or a recombinant viral vector according to the invention for the manufacture of a medicament for treating or preventing a heart disease in a primate, preferably a human.

The invention further relates to a method
(A) for treating a heart disease in a primate, wherein said method of treating or preventing a heart disease comprises the transduction of primate cardiomyocytes; or
(B) in a method of treating or preventing cardiomyopathy in a primate; or
(C) in a method of treating or preventing heart failure or chronic heart failure in a primate; or
(D) heart failure patients with reduced ejection fraction,
in a subject, comprising administering to the subject, preferably to a human, an effective amount of a pharmaceutical composition or a viral vector according to the invention.

The invention further relates to the use of viral vector according to the invention for the transduction of isolated heart tissue cells of a rat or a primate, preferably isolated cardiomyocytes.

| SEQ ID NO: | comments |
|---|---|
| 1 | Peptide 1 (see AAV BI 15.1) |
| 2 | Peptide 2 (see AAV BI 15.2) |
| 3 | Peptide 3 (see AAV BI 15.2) |
| 4 | Adeno-associated virus - 2 - VP1 Protein |
| 5 | Adeno-associated virus - 2 - VP2 Protein |
| 6 | Adeno-associated virus - 2 - VP3 Protein |
| 7 | VP1 modified with Peptide 1 |
| 8 | VP1 modified with peptide 3 |
| 9 | cap/rep plasmid modified with insert coding for peptide 1 |
| 10 | cap/rep plasmid modified with insert coding for peptide 3 |
| 11 | Primer forward |
| 12 | Primer reverse |
| 13 | Probe |
| 14 | CAG-Promoter |
| 15 | Simian virus 40 polyadenylation sequence |
| 16 | Adeno-associated virus - 2 ITR |
| 17 | Adeno-associated virus - 2 ITR |
| 18 | huMydgf protein sequence with signal peptide |
| 19 | huMydgf gene sequence |
| 20 | mutant huMydgf protein sequence with signal peptide (-RTEL) |
| 21 | mutant huMydgf gene sequence (-RTEL) |
| 22 | pAAV plasmids harbouring huMydgf |
| 23 | pAAV plasmids harbouring huMydgf-RTEL |
| 24 | Peptide 1 with stuffer |
| 25 | Peptide 3 with stuffer |
| 26 | Peptide 1 with stuffer |
| 27 | Peptide 3 with stuffer |
| 28 | AT2A2 - Sarcoplasmic/endoplasmic reticulum calcium ATPase 2 |
| | Isoform 1 protein sequence |
| 29 | AT2A2 - Sarcoplasmic/endoplasmic reticulum calcium ATPase 2 - |
| | Isoform 2 protein sequence |
| 30 | AT2A2 - Sarcoplasmic/endoplasmic reticulum calcium ATPase 2 - |
| | Isoform 3 |
| 31 | AT2A2 - Sarcoplasmic/endoplasmic reticulum calcium ATPase 2 - |
| | Isoform 4 |
| 32 | AT2A2 - Sarcoplasmic/endoplasmic reticulum calcium ATPase 2 - |
| | Isoform 5 |
| 33 | human Mydgf without signal peptide (mature protein) |
| 34 | human Mydgf without signal peptide without golgi retention signal |
| | (-RTEL) |
| 35 | Mydgf (WT) protein activity test probe compound protein sequence |
| 36 | Mydgf-RTEL protein activity test probe compound pre-protein |
| | sequence with signal peptide |
| 37 | Mydgf-RTEL protein activity test probe compound protein sequence |
| | without signal peptide |

### Examples

The following examples are provided to further illustrate certain aspects and embodiments of the present invention. The examples should however not be understood as limiting for the scope of the invention.

### 1. Materials and Methods

### 1.1 Vector Production and Quantification

AAV vector stocks were produced using CELLdiscs by equimolar transfection of BI-15.1 and BI-15.2 and AAV9 rep2/cap plasmids, phelper and single stranded (ss) CAG-eGFP or CMV-eGFP pAAV plasmids into HEK-293 cells. For transduction of human IPSC-derived cardiomyocytes, self-complementary (sc) CMV-eGFP pAAV plasmids were used as reporter transgenes. Purification was performed by polyethylene glycol precipitation, followed by iodixanol density gradient ultracentrifugation and ultrafiltration as described previously (Strobel et al, 2019). The production of recombinant AAV vectors in Sf9 insect cells was performed as described in WO 2015/158749 or US20170029464A1. The genomic titer was determined by qPCR. Briefly, viral DNA was extracted using viral nucleotide extraction Viral Express Nucleic Acid Extraction Kit (Chemicon, Cat. No. #3095). Quantitative PCR was conducted using the TaqMan Gene Expression Master Mix (4370074; Applied Biosystems) and a primer/probe set specifically binding a sequence segment of the CMV promoter that is also contained in the CAG promoter. The following primers were used:

CMV_forward:
(SEQ ID NO: 11)
5′-CGTCAATGGGTGGAGTATTTACG-3′
CMV_reverse:
(SEQ ID NO: 12)
5′-AGGTCATGTACTGGGCATAATGC-3′
CMV_probe:
(SEQ ID NO: 13)
5′-AGTACATCAAGTGTATCATATGCCAAGTACGCCC-3′

The respective plasmids were used to prepare a standard curve for quantification by serial 1:5 dilutions. For digital droplet quantification PCR was performed using the QX200 system (Bio-Rad, USA). 9 μl of viral DNA (diluted 1:1000 to 1:10⁹) were then added to 10 μL of 2× ddPCR Supermix for Probes (Bio-Rad) and 1 μL of 20× primer-probe sets specific for the target sequence of interest (here: CAG or CMV promoter). The mix was then transferred to a DG8 cartridge and droplets were generated using the Bio-Rad Droplet Generator and 70 μL of Droplet Generator Oil per well. After carefully transferring 44 μL of droplets to a 96-well plate, the plate was sealed using the Bio-Rad PX1 Plate Sealer and transferred to an Eppendorf X50s PCR Mastercycler. The cycling conditions were as follows: an initial denaturation step for 10 min at 95° C. followed by 40 cycles of 30 sec at 95° C. and 1 min annealing at 60° C. (ramping rate: 2° C./sec). Optimal annealing temperature had previously been identified by running a temperature gradient. Following a final heating step of 10 min at 98° C., the plate was cooled down to 10° C. and placed into the Droplet Reader. The data were analyzed using the QuantaSoft software (BioRad). Those sample dilutions that showed proper separation of positive and negative droplets were used for the calculation of AAV genomic titers.

1.2 AAV Binding Assay (bAb)

The presence of pre-existing total anti-capsid antibodies in serum of NHP was analyzed using a bridging immunogenicity assay format on the MSD (Meso Scale Discovery) platform. Standard Multi-Array MSD plate (L15XA-1) was coated with 5×10⁸ AAV capsids per well in AAV-formulation buffer, under shaking for 5 min at 750 rpm. After incubation at 4° C. overnight, the plate was washed three times. Blocking was performed using blocking solution (3% Blocker A (R93BA-2, MSD) in PBS) for 1 hour at room temperature (rt) followed by washing. Serum of NHPs, IVIG (Kiovig; Baxter) as control or mouse A20 (Progen; 61055) as AAV2 coating control were prepared in serial 1:2 dilutions in 1% blocker A solution and incubated for 1 hour at rt on coated AAV capsids. After washing, amount of bound IgG₁₋₃ was detected by incubation with anti-human NHP IgG1-3 (MSD; D20JL-6) or anti mouse IgG (MSD; R32AC-1) control for 1 hour at rt following washing and addition of 2×MSD Read Buffer. Within 5 min electrochemiluminescence was detected by the MSD Sector Imager 6000 using Discovery Workbench software version 3.0.18. Values of PBS-coated wells for each individual serum (or IVIG) dilution was subtracted from each individual AAV coated sample. Relative IgG₁₋₃ MSD signal was normalized to A20 values. The serum dilution that mediated 50% of the maximum value of the IgG1-3 signal was reported as the bAB-titer.

1.3 Neutralizing Assay (nAb)

In addition to anti-capsid antibodies and potentially neutralizing antibodies, transduction inhibition assays may detect non-antibody neutralizing factors present in NHP sera. 96-well plates were seeded with 5×10⁴ HEK293 cells per well for 24 hr. Recombinant BI-15.1 or BI-15.2 or AAV2 (with anti-FITC) was diluted in Dulbecco's modified Eagle's medium (DMEM; Invitrogen Life Technology, Carlsbad, CA) supplemented with 10% fetal calf serum (FCS) and incubated with 2-fold serial dilutions (1:2 to 1:1024) of NHP serum samples for 30 minutes at 37° C. Subsequently, the serum-vector mixtures corresponding to 25000 VG/cell (AAV2-NRGTEWD and AAV2-ESGHGYF) or 2500 VG/cell (AAV2), were added to plated cells and incubated in DMEM-10% FCS for 72 hours at 37° C. and 5% CO2. Each mix was performed in triplicate. The supernatant was transferred to 96-well plates and then the amount of anti-FITC was determined by anti-FITC ELISA. Transduction efficiency was measured as relative counts per well. The neutralizing titer was reported as the highest serum dilution that inhibited the rAAV transduction by 50% compared with the control without serum.

### 1.4 Electron Microscopy Analysis of Vector Stocks

CryoTEM and nsTEM analysis was performed at Vironova (Stockholm, Sweden). AAV samples were diluted to a suitable “on grid” concentration. For cryoTEM, 3 μl of each sample were applied onto a continuous or holey carbon EM grid and subsequently plunge-frozen in liquid ethane using the FEI Vitrobot™. The grids were imaged using a JEOL JEM-2100F or Philips CM200 field emission gun transmission electron microscope run at 200 kV accelerating voltage. For nsTEM samples were applied onto a suitable continuous carbon grid, washed with water, and negatively stained using 2% uranyl acetate (UAc) or other suitable stains. The grids were imaged using a MiniTEM™ run at 25 kV accelerating voltage. Representative areas were imaged at both low and high magnification. A full set of images was acquired only on the grid showing suitable on grid concentration, particle distribution and image contrast. EM grids were prepared in accordance with the SOP V0149, Sitting drop sample preparation for negative stain transmission electron microscopy (nsTEM). Automatic detection and classification were performed on 1.5 micrometer FOV images to generate morphological classification and size distribution plot and statistics of the found particles.

### 1.5 Analysis of Transduction on Human Cardiomyocytes

The human induced pluripotent stem cell (hiPSC, line SFC086-03-01) was obtained from the IMI-StemBANCC project (Morrison et al, 2015) (http://stembancc.org). hiPSCs were seeded on culture plates coated with growthfactor-reduced Matrigel (Corning, NY, USA). hiPSCs were maintained at 37° C., 5% CO2 using Essential8™ flex medium (Thermo Fisher Scientific, Waltham, MA, USA) supplemented with 100,000 U/l penicillin and 100 mg/l streptomycin (Thermo Fisher Scientific). The cells were passaged with 0.5 mM EDTA (Thermo Fisher Scientific) every 3 to 5 days, after reaching a confluence of approximately 70%.

Differentiation of hiPSCs into cardiomyocytes was performed based on a protocol published by (Burridge et al, 2014) with modifications. In brief, 1×10⁴ hiPSCs/cm² were seeded on growth-factor-reduced Matrigel coated culture plates in E8-flex medium supplemented with 10 μM Y-27632 (Sigma Aldrich, Merck KGaA, Darmstadt Germany), medium was changed to E8-flex medium daily for 4 days. Differentiation was induced by medium replacement to CMD medium (RPMI 1640 medium (Thermo Fisher Scientific) supplemented with 0.25% Bovine Albumin Fraction V (Thermo Fisher Scientific) and 0.21 mg/mL L-ascorbic acid sphosphat (Wako Chemicals, Osaka, Japan)) supplemented with 5.5 μM CHIR99021 (Sigma Aldrich) (Day 0 of differentiation). After 48 h medium was replaced with CMD medium supplemented with 5 μM IWP2 (Merck Millipore, Merck KGaA). After 48 medium was renewed every other day with CMD medium. Cell were cryopreserved at day 14 of differentiation using Multi Tissue Dissociation Kit 3 (Miltenyi Biotec, Bergisch Gladbach, Germany) according to the manufacturer's protocol. Cardiomyocytes were purified using the noncardiomyocyte depletion step of the PSC-Derived Cardiomyocyte Isolation Kit (Miltenyi Biotec) according to the manufacturer's protocol.

Cryopreserved cardiomyocytes were thawed on fibronectin (0.8 μg/cm²; Sigma Aldrich) coated culture flasks in RB+ medium (RPMI 1640 medium supplemented with 1:50 B27 supplement (Thermo Fisher Scientific)) and 10 μM Y-27632. After 24 h the medium was renewed with RB+ medium. The day after cells were detached using StemPro™ Accutase™ (Thermo Fisher Scientific) and 1.5×10⁴ cells per well were seeded on a fibronectin coated 96 half area well plate (Greiner BioOne, Frickenhausen, Germany) in RB+ medium. PBS was added to peripheral wells. After 48 hours spheroids were washed with maturation medium (MM; DMEM no glucose, 10 mM HEPES, 1 mM nonessential amino acids (all Thermo Fisher Scientific), 2 mM L-carnitine, 5 mM creatine, 5 mM taurine, 1:100 ITS+3 Liquid Media Supplement (all Sigma Aldrich) (Drawnel et al, 2014)) and cultivated in MM. MM was renew after 72 h. For transduction cells were seeded in 96 well half area microplate (Greiner, Bio-One, Frickenhausen, Germany) 10 μl containing 2×10⁹ vg of the indicated vector were applied and plates were incubated for 48 hours before picture of transduction efficacy were assessed using the Opera Phenix High-Content Screening System (Perkin Elmer, Waltham, Massachusetts).

### 1.6 Analysis of Vector Biodistribution and Gene Expression

Tissue samples were flash frozen in liquid nitrogen immediately after dissection. For DNA and RNA isolation, samples were homogenized in 900 μL RLT buffer (79216, Qiagen), using a Precellys 24 homogenizer and ceramic bead tubes (KT03961-1-009.2, VWR) at 6000 rpm for 30 sec. Samples were immediately placed on ice. 350 μL Phenol-chloroform-isoamyl alcohol (77617, Sigma Aldrich) were then added to 700 μL homogenate in a phase lock gel tube and mixed by shaking. Following centrifugation for 5 min at 16000×g, 350 μL Chloroform-isoamyl alcohol (25666, Sigma-Aldrich) were added and the mixture was shaken again. After 3 min of incubation at rt and centrifugation for 5 min at 12000×g, the upper phase was collected and pipetted into a deep well plate placed on dry ice. After processing of all samples, DNA and RNA were purified, using the AllPrep DNA/RNA 96 kit (80311, Qiagen) as per instructions, including the optional “on-column DNase digestion” step. RNA from cell cultures was isolated by pelleting cells, followed by lysis in 350 μL RLT buffer and purification using the RNeasy mini kit (74104, Qiagen). Integrity of RNA was confirmed by Fragment Analyser (Agilent). For biodistribution analysis, AAV vector genomes were detected using extracted DNA and a standard curve generated by serial dilutions of the respective expression plasmid. Taqman runs were performed on an Applied Biosystems ViiA 7 Real-Time PCR System. For gene expression analysis, equal amounts of RNA were reversely transcribed to cDNA using the High-capacity cDNA RT kit (High capacity cDNA Archive Kit; #4322169, Applied Biosystems) as per instructions. qRT-PCR reactions were then set up using the TaqMan Gene expression Master Mix (#4370074, Applied Biosystems) and primers specifically binding the eGFP or Mydgf (Hs00384077_m1, Thermo Fisher) gene. Expression was normalized to RNA polymerase II (RNA POLII gene ID; XM_015437398.1 or Rn01752026_m1, Thermo Fisher) housekeeper expression.

### 1.7 Immunohistochemistry

Tissue samples of mouse, rat and NHP origin were fixed in 4% PFA and paraffin embedded (formalin fixed and paraffin embedded, FFPE). 3 μm thick sections of FFPE tissue on super frost plus slides were deparaffinised and rehydrated by serial passage through changes of xylene and graded ethanol for immunohistochemistry staining. Antigen retrieval was performed by incubating the sections in Leica Bond Enzyme solution (Bond Enzyme Pre-treatment Kit, Cat #35607) for 5 minutes. Sections were incubated with an anti-GFP antibody (abcam, ab290, rabbit polyclonal). The antibody was diluted (1:1500) with Leica Primary Antibody Diluent (AR9352; Leica Biosystems, Nussloch, Germany) and incubated for 30 min at room temperature. Bond Polymer Refine Detection, (Cat #37072) was used for detection (3,3′ Diaminobenzidine as chromogen, DAB) and counterstaining (hematoxylin). Staining was performed on the automated Leica IHC Bond-III platform (Leica Biosystems, Nussloch, Germany). Microscopic assessment of samples was conducted with a Zeiss AxioImager M2 microscope and ZEN slidescan software (Zeiss, Oberkochen, Germany). For staining of Mydgf a pre-treatment step with incubation in citrate for 30 min at 95° C. was performed and anti-Mydgf antibody (proteintech, Art.: 11353-1-AP, rabbit IgG polyclonal) diluted 1:500 (Diluent Cat #35089; Leica Biosystems, Nussloch, Germany) was used for detection. Staining was performed using standard Leica staining protocol for anti-rabbit IgG.

### 1.8 Image Analysis

For quantitative analysis anti-GFP stained sections of the heart were scanned with an Axio Scan.Z1 whole slide scanner (Carl Zeiss Microscopy GmbH, Jena, Germany) using an 20× objective (0.22 μm/px) in bright field illumination. The area of the anti-GFP-positive tissue was identified with the image processing software HALO 3.0 using the area quantification module 1.0 (Indica Labs, Corrales, NM, USA). Color deconvolution was used to split signals of hematoxylin and DAB. A threshold was manually optimized to identify positive areas aₚ in the DAB channel, while having minimized response to background signal. The total tissue area A was obtained from areas with markedly higher optical density than the background. The fraction of stained area f was obtained by normalizing the DAB positive area aₚ by the total tissue area A, i.e. f=aₚ/A. Identical color deconvolution, threshold and tissue area detection settings were used throughout this study to ensure comparability.

1.9 Expression and Detection of huMydgf by Western Blotting

For expression in mammalian cells, 2.5 μg pAAV plasmids harbouring huMydgf (SEQ ID NO:22) or huMydgf-RTEL (SEQ ID NO:23) under control of the CAG-promoter were transfected into HEK293 cells obtained from Thermo (#11631-017) using Lipofectamine3000 transfection reagent (Thermo #L3000001). Cells were grown in DMEM supplemented with 10% fetal bovine serum. For transient transfection, 1×10⁶ cells were plated in growth medium in 6 well plates, 16 h before transfection. After 48 h, conditioned medium was carefully collected leaving the cell layer intact, any cells in the collected medium were spun down by centrifugation. The cells were removed from the plate via cell scraper in 1 ml of cold PBS. Cells were pelleted at 400 relative centrifugal force, and PBS was removed. The pelleted cells were lysed with 100 μl of RIPA buffer with Protease Inhibitor Cocktail (Thermo #89901; #78438). Lysate protein concentration was determined via BCA Assay (Thermo #23225). For immunoblotting 50 μg lysate protein or 30 μl undiluted conditioned medium was boiled for 5 min at 95° C. in 4× Laemmli buffer with 2.5% β-mercaptoethanol (Biorad #1610747). PVDF membrane was blocked with 5% dry milk. 1 μg/ml anti-MYDGF Antibody (R&D #AF1147) was incubated over night at 4° C. Secondary anti-goat antibody (Dianova #705-035-003) was used at 100 ng/ml for 1 h at room temperature.

### 1.10 Animals

All animal procedures were performed in accordance with the guide for the care and use of laboratory animals published by the German animal protection code and approved by local authorities (Regierungspräsidium Tübingen, Germany). For tropisms studies AAV vectors were administered to 8-10 weeks old female C57BL/6 mice, whistar kyoto rats (WKY/KyoRj) or sprague dawley rat and non human primates (Macaca fascicularis, mauritian origin).

### 1.11 AAV Application Protocols

For tropisms studies C57BL/6 mice, whistar kyoto rats or sprague dawley rat were injected under anaesthesia (3.5% isofluran) with a volume of 5 ml/kg bodyweight into the tail vein. All NHPs (Macaca fascicularis, mauritian origin) were prescreened for neutralising antibodies and selected individuals were injected with a volume of 1 ml/kg bodyweight with an infusion rate of 3 ml/min into the arm vein.

All graphs and statistics were created using Prism8 (GraphPad Software, San Diego, USA). One-way ANOVA with Dunnett's multiple comparison test or unpaired, two-tailed student T-test was used.

### 1.12 Mouse Model of Myocardial Infarction.

Myocardial infarction (MI) was induced as described in Korf-Klingebiel et al. 2015. MI was induced in 9-10 week old FVB/N mice by transient left anterior descending coronary artery (LAD) ligation. Mice were pretreated with 0.02 mg kg⁻¹ atropine subcutaneously (SC) (B. Braun) and 2 mg kg⁻¹ butorphanol SC (Pfizer). Mice were ventilated with 3-4% isoflurane (Baxter) via face mask. After oral intubation, anesthesia was maintained with 1.5-2% isoflurane. Left thoracotomy was performed, and the LAD was ligated with a slipknot (ischemia), which was removed 1 h later (reperfusion). In control mice, ligature was not tied around the LAD (sham operation). High-resolution two-dimensional transthoracic echocardiography in mice sedated with 1-2% isoflurane was performed (linear 20-46 MHz transducer MX400, Vevo 3100, VisualSonics). LV end-diastolic area (LVEDA) and LV end-systolic area (LVESA) from the long-axis parasternal view was recorded. Fractional area change (FAC) was calculated as [(LVEDA-LVESA)/LVEDA]×100. LV pressure-volume loops were recorded with a 1.4 F micromanometer-tipped conductance catheter inserted via the right carotid artery (SPR-839, Millar Instruments). For anesthesia, mice were pretreated with 2 mg kg⁻¹ butorphanol SC and ventilated with 4% isoflurane via face mask. After oral intubation the mice were treated with 0.8 mg kg⁻¹ pancuronium intraperitoneally (IP) (Actavis) and anesthesia was maintained with 2% isoflurane. Steady-state pressure-volume loops were measured at a rate of 1 kHz and analyzed with LabChart 7 Pro software (ADInstruments). Osmotic minipumps (Alzet) filled with recombinant Mydgf, Mydgf-RTEL, or diluent (PBS) were placed in a SC interscapular pocket just before coronary reperfusion. Model 1007D was used for 7 d infusion (pumping rate 0.5 μl/h, filled with 10 μg of respective protein per 12 μl).

### 1.13 Recombinant Mydgf and Recombinant Mydgf-RTEL

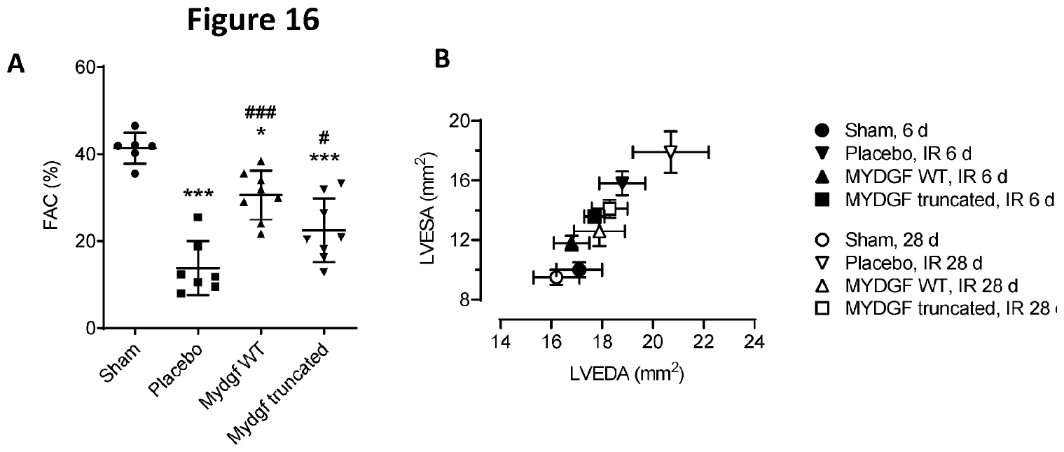

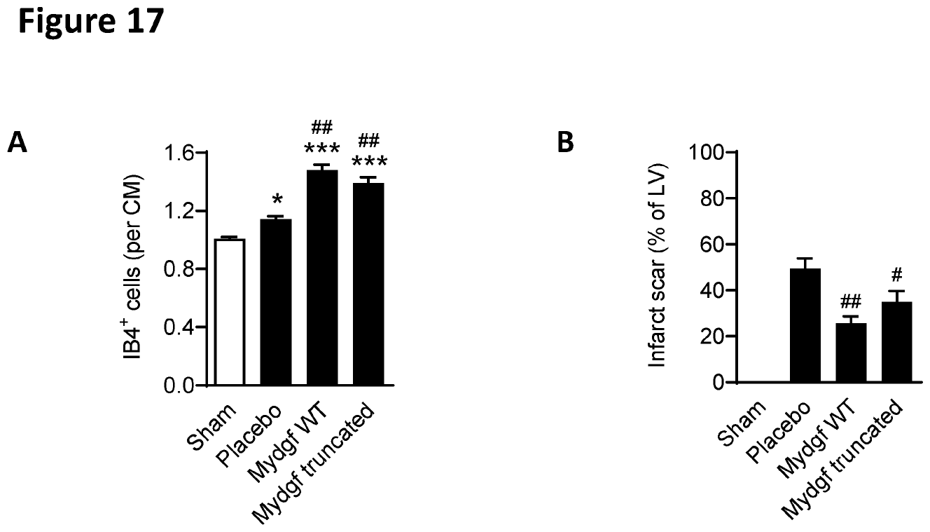

The first protein used in the experiments of FIGS. 16 and 17 was recombinant Mydgf expressed in E. coli with the following sequence:

(M)VSEPTTVAFDVRPGGVVHSFSHNVGPGDKYTCMFTYASQGGTNEQWQ
MSLGTSEDHQHFTCTIWRPQGKSYLYFTQFKAEVRGAEIEYA-
MAYSKAAFERESDVPLKTEEFEVTKTAVAHRPGAF-
KAELSKLVIVAKASRTEL

The protein was expressed in E. coli and recovered/purified by a process described in the following scheme:
1. Cell disruption+inclusion body (IB) recovery, IB solubilzation+refolding
2. diafiltration
3. AIEC capture (YMC Q75)
4. HIC Intermediate (Capto Phenyl)
5. UFDF (ultrafiltration/diafiltration)
6. approx 100 mg purified product

The second protein used in the experiments of FIGS. 16 and 17 was recombinant Mydgf-RTEL with the following sequence:

MGWSLILLFLVAVATRVLS
HHHHHHAGSENLYFQGVSEPTTVAFDVRPGGVVHSF-
SHNVGPGDKYTCMFTYASQGGTNEQWQMSLGTSEDHQHFTCTI-
WRPQGKSYLYFTQFKAEVRGAEIEYA-
MAYSKAAFERESDVPLKTEEFEVTKTAVAHRPGAFKAELSKLVIVAKAS

The MYDGF (-RTEL) protein used in FIGS. 16 and 17 is depicted above with a signal peptide (MGWSLILLFLVAVATRVLS) followed by His tag, linker, and then TEV cleavage site. The signal peptide is cleaved during expression resulting in a mature protein sequence shown under the signal peptide sequence above. This protein was expressed in HEK 293E cells. DNA encoding the protein referenced above was synthetically produced with codon optimization for mammalian cell expression and cloned into pTT5 (Invitrogen Carlsbad, CA) at the restriction sites HindIII/NotI by standard methods. Each transfection was 1 L transfection size using PolyPlus PEI as transfection method. Cells were harvested on day 4 post transfection and the supernatant was collected by centrifugation at 9300×g for 30 min at 4 C. The protein was purified from supernatant by Ni-NTA batch binding overnight at 4 C. Eluted protein was characterized by SDS PAGE gel and analytical size exclusion chromatography. While the protein has a TEV cleavage site the protein was not cleaved and still contained the His tag.

### 1.14 Scar Size Measurement

Scar size measurement was conducted as described in Korf-Klingebiel et al, 2015. To measure scar size 28 days after reperfusion, the left ventricles were embedded in OCT compound (Tissue-Tek), snap-frozen in liquid nitrogen, and stored at −80° C. 6 μm sections from basal, midventricular, and apical slices were cut and them stained with Masson's trichrome and analyzed on light microscopy (Zeiss Axio Observer.Z1). Scar size was calculated as the average ratio of scar area to total LV area in basal, midventricular and apical sections.

### 1.15 Capillarization

For the evaluation of capillarization in the infarct border zone 28 days after ischemia/reperfusion, 6 μm cryosections were cut from midventricular slices. For fluorescent stainings, cryosections were stained with rhodamine-labeled wheat germ agglutinin (WGA, Vector Laboratories) to visualize cardiac myocyte borders and interstitial matrix and fluorescein-labeled GSL I isolectin B4 (IB4, Vector Laboratories) to visualize capillaries. IB4-positive cells per cardiomyocyte were calculated using axio vision software.

### 1.16 Activity Assay

Scratch Assay in human coronary artery endothelial cells (HCAEC). HCAECs are seeded in a density of 55.000-60.000 HCAECs per well in a 24 well plate in EGM2 Medium with 10% FCS in a total volume of 1 ml per well. 24 hours after seeding (cells need to be confluent), the medium is exchanged to 1 mL MCDB medium containing 2% FCS each well and incubated for 3-4 hours. After incubation the monolayer is scratched with a yellow pipet tip (200 μl) in each well (use the tip vertical to ensure the scratch is big enough). The cells are then washed once with MCDB medium with 2% FCS and subsequently each well 1 mL of fresh medium (MCDB/2% FCS) is added. Subsequently the cells are stimulated with a protein probe at different concentrations each well with a starting concentration and a serial dilution of 1:1.5. Directly after treatment at T=0 h, a picture has to be taken from all wells at a microscope (e.g. Zeiss Axio Observer Z1 with 50× magnification (5× objective) with phase contrast setting). Ideally the pictures are taken from the middle of the wells, as the optimal contrasts are seen there. The plates are then incubated at 37° C. After incubation time of 16 hours (T=16 h) again pictures of each well have to be taken as described before. For determination of the activity the recovery in the assay is calculated by measuring the cell free area (e.g. with axiovision software or ImageJ) in 0 h pictures and 16 h pictures. Recovery (%) is calculated as [(cell free area at 0 h-cell free area at 16 h)/cell free area at 0 h]×100.

### 2. Results

### 2.1 Vector Production

To evaluate the potential of BI-15.1 and BI-15.2 as vectors targeting cardiomyocytes in rats and non-human primates, vector batches ranging from 0.6-1.2×10¹⁵ vg (summarized in Table 2) were produced in cell discs as previously described (Strobel et al, 2019). Vectors were produced to express enhanced green fluorescence protein (eGFP) under control of either 1.) cytomegalovirus early enhancer+chicken β-actin (CAG) promoter, or under control of 2.) cytomegalovirus (CMV) promoter.

TABLE 2
Summary of large scale AAV preparations and analytics.

| | Insertion | | | | |
|---|---|---|---|---|---|
| | sequence | Expression | Number of | Total yield | Final titer |
| ID | (AAV2-R588) | cassette | cell discs | (VG) | (VG/ml) |
| AAV BI-15.1 | NRGTEWD | ssAAV2-CAG-eGFP | 14 | 1.2x10e15 | 2.2x10e13 |
| AAV BI-15.2 | ESGHGYF | ssAAV2-CMV-eGFP | 14 | 6.1x10e14 | 3.7x10e13 |

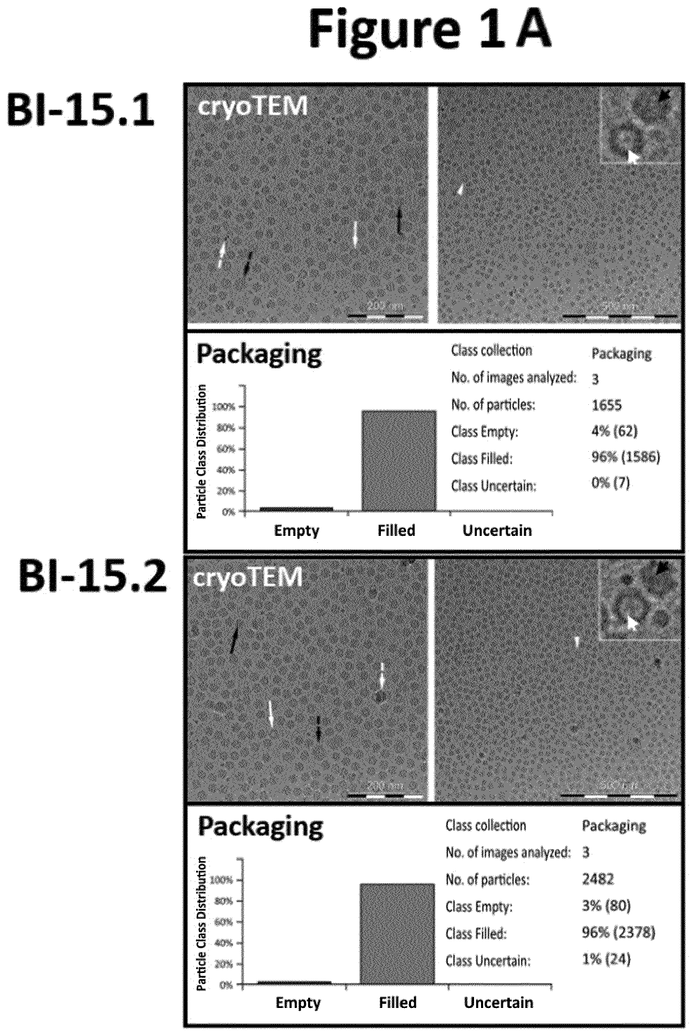

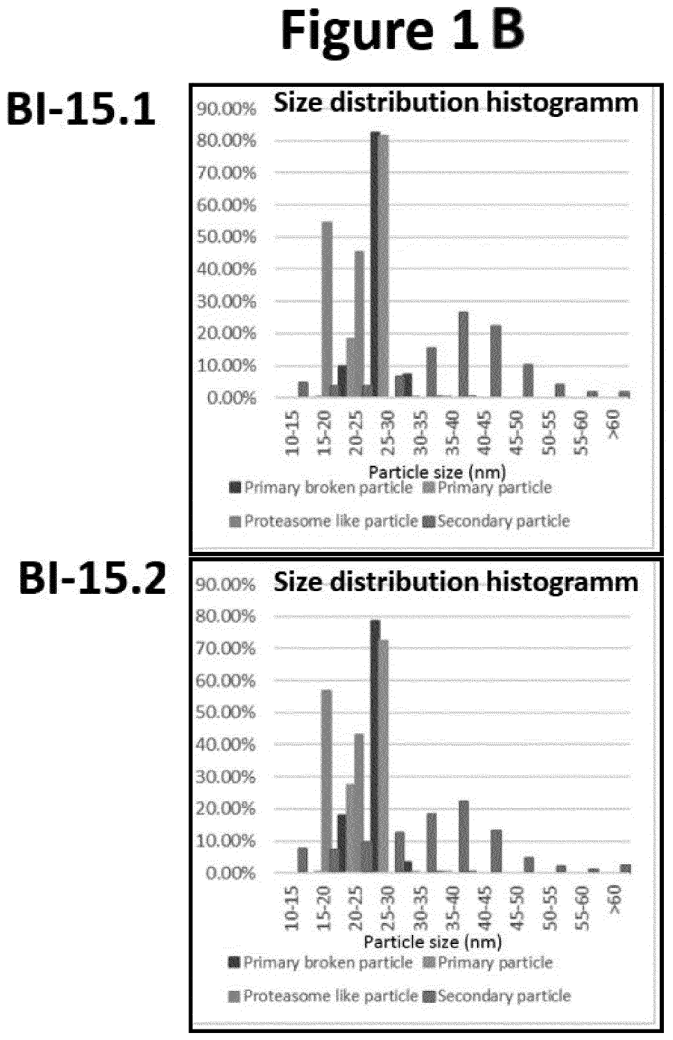

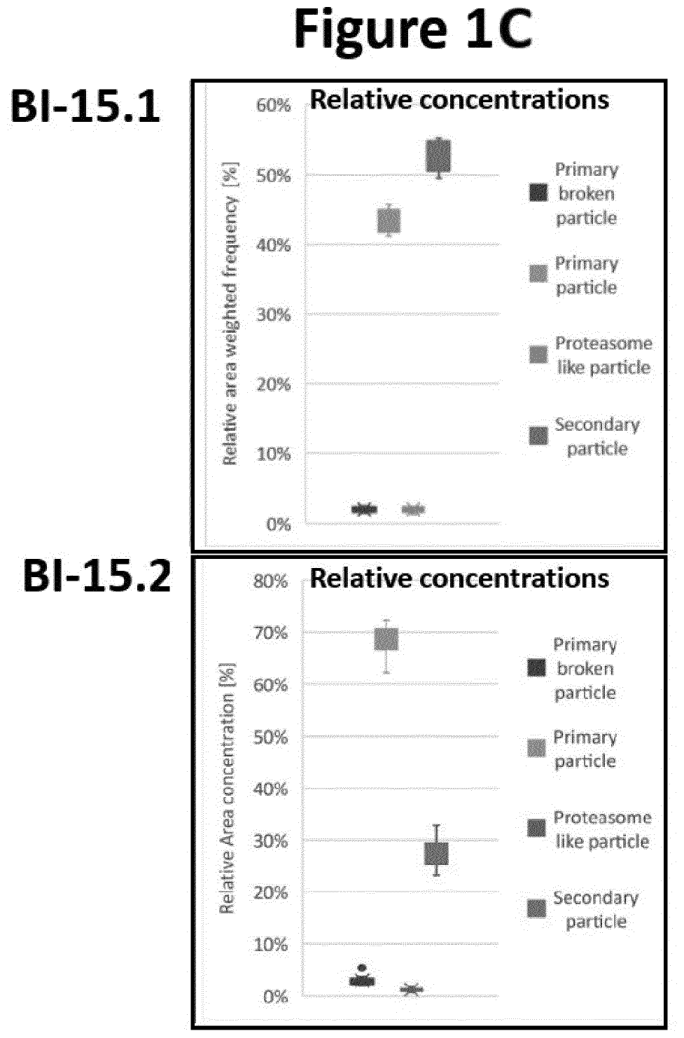

The quality of the HEK-based vector preparations was analyzed by transmission electron microscopy (TEM) regarding purity, aggregation, capsid assembly as well as packaging ratios. CryoTEM analysis showed a high concentration of evenly distributed full AAV particles with no detection of particle aggregates or minor clustering (FIG. 1A upper panel of BI-15.1 and upper panel of BI-15.2). Image based internal density analysis showed a packaging ratio of ˜96% for both vector preparations (FIG. 1A lower panel for each capsid variant each). In addition, negative stainTEM (nsTEM) was used to assess the sample purity and particle classification. Here, BI-15.1 was composed of ˜43% of primary AAV particles and ˜57% primary broken particles. BI-15.2 was composed of ˜70% primary AAV particles with a corresponding lower amount of primary broken particles (˜30%). For both vector batches, broken AAV particles and small sized particles, that are likely to be proteasomes, were reported to be below 5%.

### 2.2 Vector Distribution in Mice

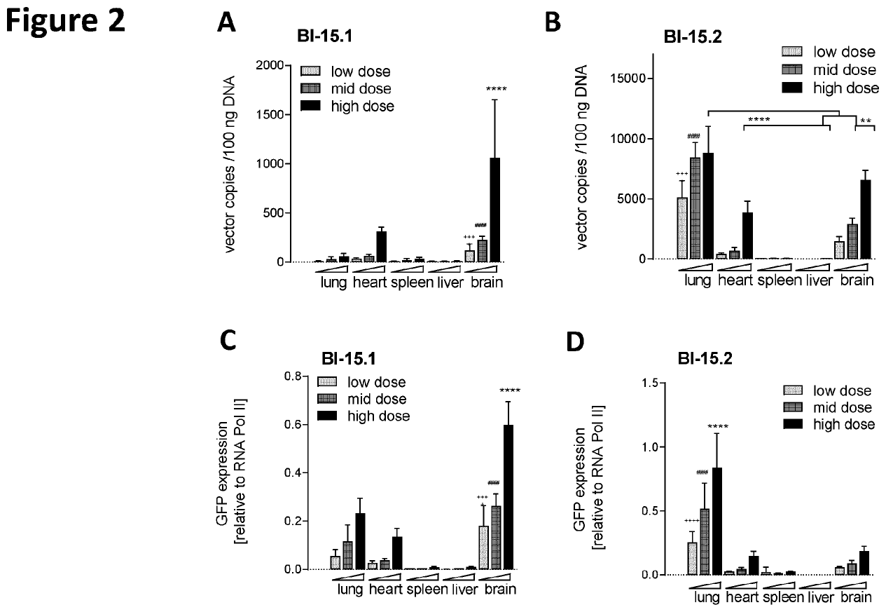

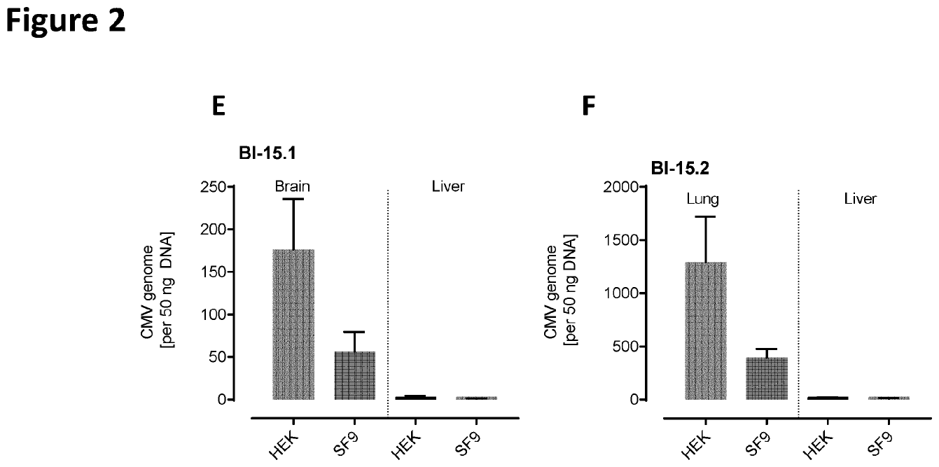

To confirm the previously published capsid-mediated tropism and gene delivery properties of BI-15.1 and BI-15.2, vectors were intravenously injected into female C57BL/6J mice at three different doses (low dose: 5×10¹² vg/kg body weight (BW), mid dose: 1×10¹³ vg/kg BW, high dose: 5×10¹³ vg/kg BW). Three weeks after vector administration, vector distribution was determined in tissue samples of the brain, lung and a panel of off-target tissues. Quantification of viral genomic copy numbers confirmed significant capsid mediated homing of BI-15.1 to the brain in all dose cohorts while only low vector copy numbers were detected in off-target organs (FIG. 2A). Concomitantly, eGFP gene expression levels analyzed by quantification of RNA transcripts were significantly and specifically stronger in whole brain lysates compared to off-target tissues (FIG. 2C). In BI-15.2 injected mice, a significant capsid-mediated vector homing to lung tissue was confirmed by vector genome-based tissue distribution analysis. Lower amounts of vector genomes were detected in brain and cardiac tissue and only weak to undetectable vector copy number signals were present in various other control tissues (FIG. 2BA). In line with these data, a statistic significant and strong and dose-dependent BI-15.2 mediated reporter gene expression was confirmed by quantification of eGFP transcripts in the lung (FIG. 2D). To confirm bioactivity of Sf9 produced BI-15.1 and BI-15.2, 10e11 vg/mouse were intravenously injected into female C57BL/6J mice. Two weeks after vector administration, vector distribution was determined in tissue samples of the brain, lung and liver was analyzed. Quantification of viral genomic copy numbers confirmed significant capsid mediated homing of BI-15.1 to the brain while only low vector copy numbers was detected in the liver FIG. 2E). Quantification of viral genomic copy numbers confirmed significant capsid mediated homing of BI-15.2 to the lung while only low vector copy numbers were detected in the liver (FIG. 2F). Taken together, these results confirm bioactivity for HEK as well as Sf9 produced BI-15.1 and BI-15.2. All vectors showed the previously reported capsid-mediated targeting properties of BI-15.1 and BI-15.2 in mice and hence qualify the vectors for application to larger animal models.

### 2.3 Vector Distribution in Rats

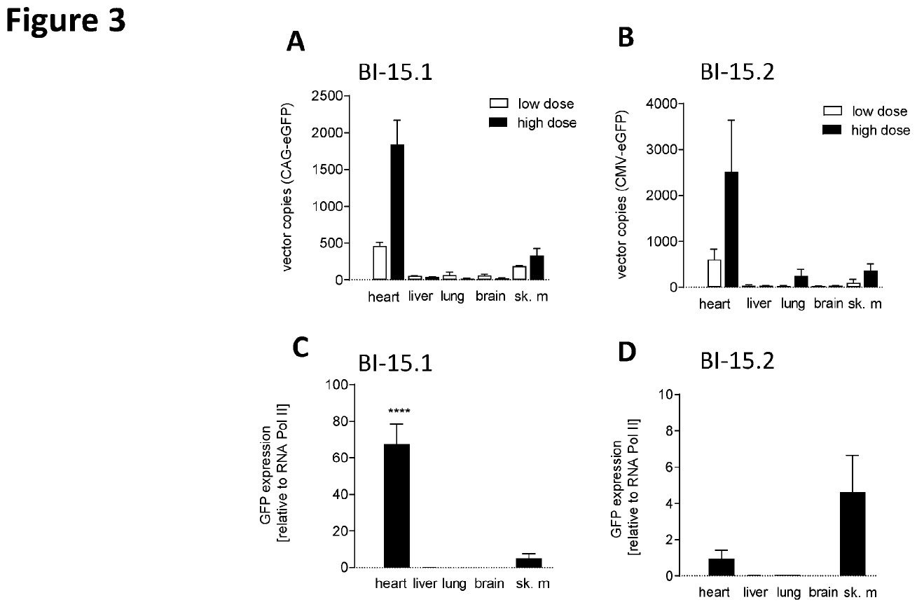

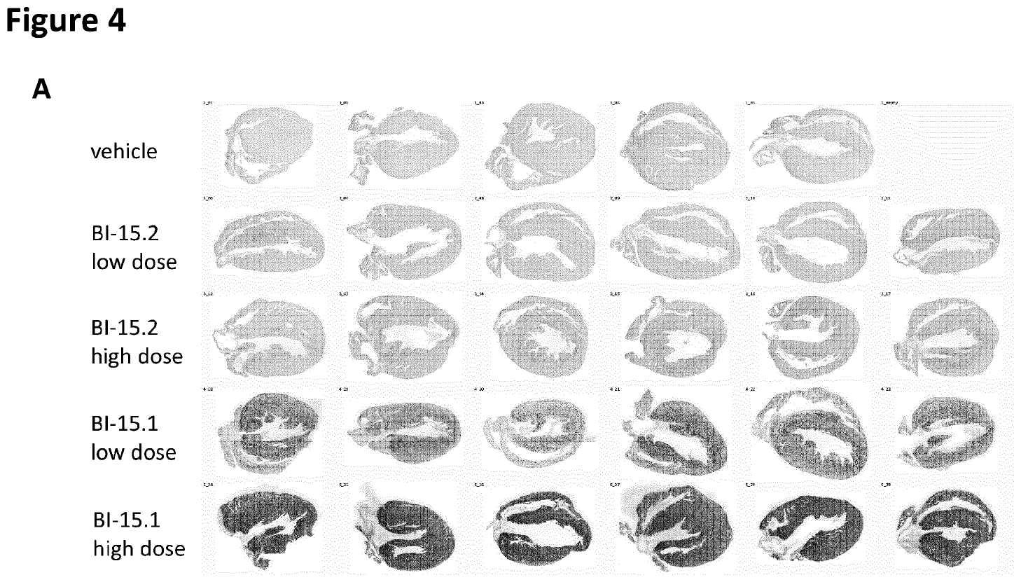

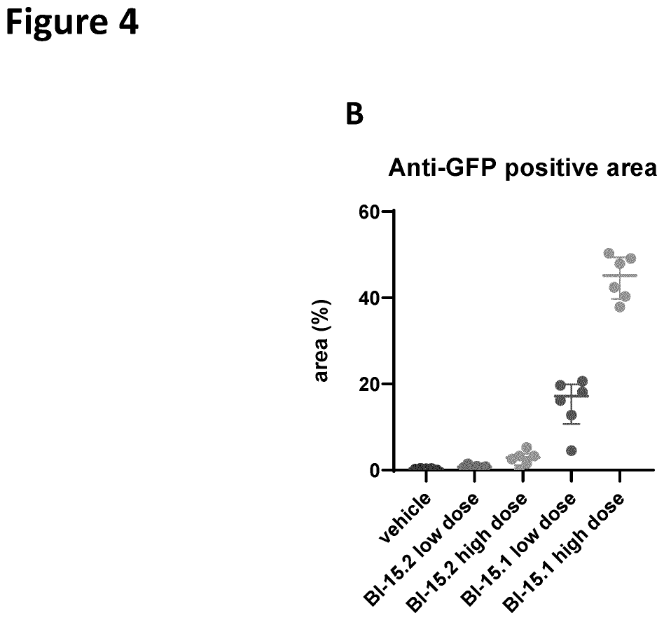

To further analyze the biodistribution and gene expression profile of BI-15.1 and BI-15.2, WKY/KyoRj rats were intravenously injected with 1×10¹³ and 5×10¹³ vg/kg. After 21 days, BI-15.1 and BI-15.2 DNA quantification of viral distribution and RNA analyses of gene expression showed significant and dose-dependent capsid mediated vector homing to cardiac tissue (FIG. 3A, B). Importantly, BI-15.1 capsid mediated cardiac homing strongly correlated with CAG-driven cardiac gene expression as shown by RNA-expression pattern (FIG. 3C). This was further confirmed by dose-dependent increase in immunohistological eGFP signal (FIG. 4A) and area quantification (FIG. 4B) assessed in whole heart sections. In contrast to a strong cardiac-specific eGFP-expression mediated by BI-15.1, BI-15.2 vector mediated expression was higher in skeletal muscle followed by the heart (FIG. 3D). This shows that CAG drives eGFP gene expression more efficiently in cardiac than in skeletal muscle, while CMV-driven expression is stronger in skeletal muscle. However, a dose dependent increase in immunohistological eGFP signal (FIG. 4A) and area quantification of eGFP positive stained cardiac area (FIG. 4B) was observed for BI-15.2 vectors.

### 2.4 Comparison of Performance of BI-15.1 Versus AAV9

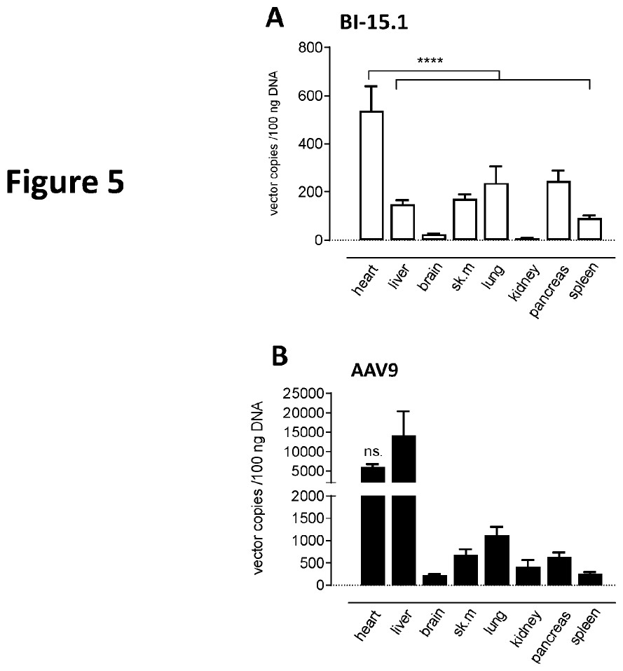

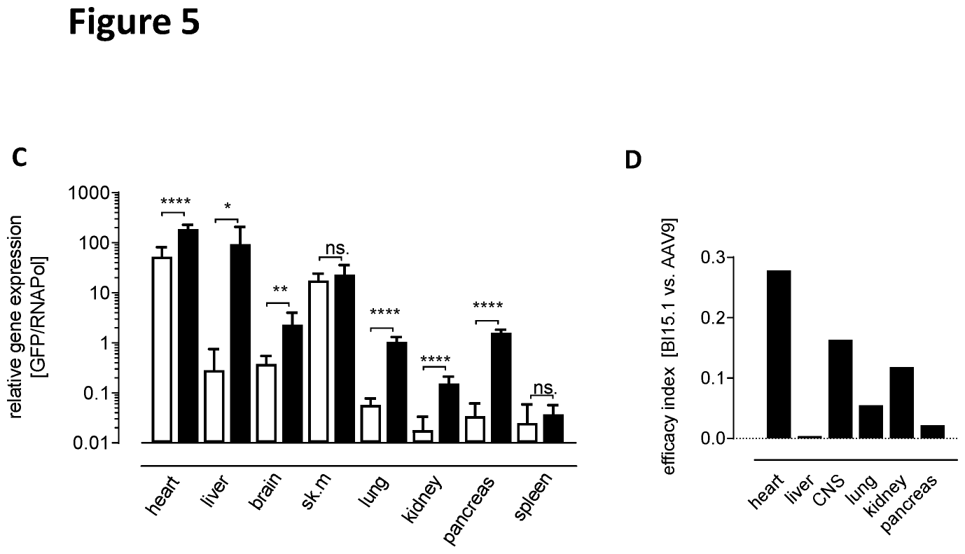

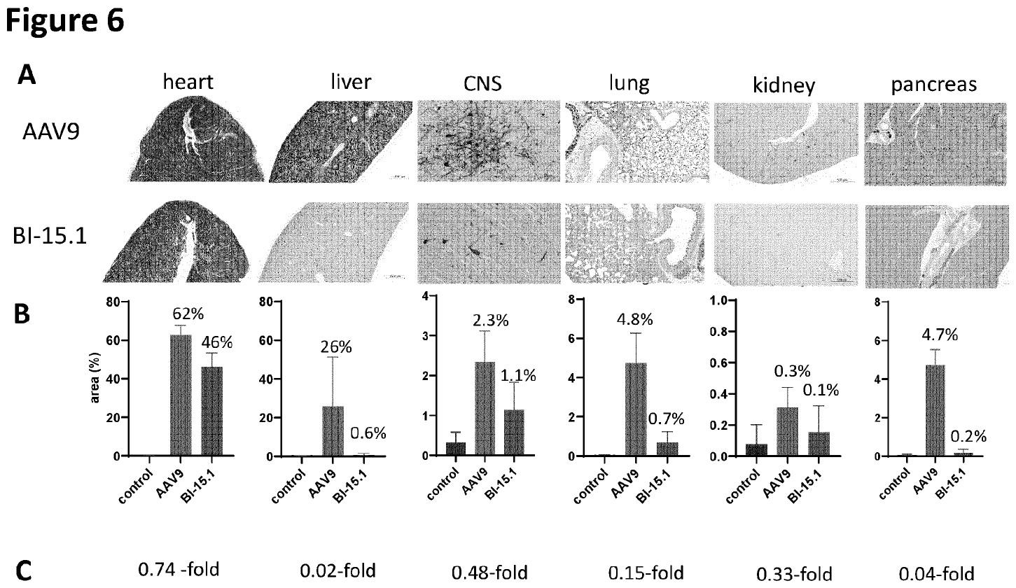

The performance of BI-15.1 and AAV9, the current standard for cardiac gene transfer in pre-clinical models, was compared. 3 weeks after i.v. systemic vector administration to Sprague Dawley rats (vector dose 3×10¹³ vg/kg BW) DNA quantification confirmed BI-15.1 capsid mediated cardiac tropism. Significantly higher viral DNA copy numbers were detected in the heart compared to liver, brain, skeletal muscle lung kidney pancreas and spleen (FIG. 5A). This shows that BI-15.1 capsid-mediated cardiac tropism is conserved in two different rat strains. These results confirm the published broad tropism of AAV9 (FIG. 5B). In addition, higher DNA copy numbers in various tissues suggests a longer half-life time of AAV9 in vivo compared to BI-15.1. This systemic persistence may lead to stronger off-target effects e.g. liver transduction. The biodistribution patterns of BI-15.1 and AAV9 correlate with vector mediated gene expression, shown by RNA analysis (FIG. 5C), immunohistochemical eGFP staining (FIG. 6A), and area quantification (FIG. 6B). Comparing vector homing, AAV9 showed higher DNA copy numbers in the heart (11.3-fold). While the RNA expression level of BI-15.1 still reached 28% of AAV9 (FIG. 5D). This indicates a higher DNA to RNA expression ratio for BI-15.1. In line with this, the efficacy index also shows a favorable expression profile of BI-15.1 vectors in various tissues (FIG. 5D). These findings were further confirmed by the area quantification-based efficacy index. BI-15.1 vector mediated expression in heart reached 74% of AAV9, while significant detargeting in various off-target tissues is shown (FIG. 6C).

### 2.5 Vector Distribution in Non-Human Primates

Lack of tropism translatability across different species is one of the major issues for AAV-based gene therapy. To explore the ability of BI-15.1 and BI-15.2 vectors to specifically deliver genes to cardiac tissue in non-human primates, we performed biodistribution studies in adult cynomolgus macaques using the dosages set out below in Table 3.

TABLE 3
Summary of cynomolgus monkeys with injected AAV-variant and dose.

| | | | Bodyweight at | | nAb | bAb |
|---|---|---|---|---|---|---|
| ID | Vector | Age [yr] | injection [kg] | Dose (vg/kg BW) | Titer | Titer |
| AJ574 | — | 16 | 6.1 | — | 1:8 | 1:64 |
| AJ562 | AAV BI-15.1 | 16 | 7.8 | 1 × 10e13 | neg. | 1:2 |
| AY895 | AAV BI-15.1 | 12 | 5.2 | 1 × 10e13 | neg. | neg. |
| AZ013 | AAV BI-15.1 | 12 | 7.5 | 1 × 10e13 | neg. | neg. |
| AJ249 | AAV BI-15.2 | 16 | 4.7 | 1 × 10e13 | neg. | neg. |
| AY613 | AAV BI-15.2 | 12 | 5.1 | 1 × 10e13 | neg. | neg. |

The presence of antibodies against AAVs can have important implications for pre-clinical experiments. Therefore, the serum of all animals included in the study were tested for presence of neutralizing antibodies, as well as AAV binding IgG₁₋₃ antibodies. The nAb titer is reported as the highest serum dilution that inhibited the rAAV transduction by 50% compared to the control without serum. The serum dilution that mediated 50% of the maximum value of the IgG1-3 signal was reported as the bAb-titer. Table 3 summarizes the titer of AAV neutralizing or binding antibodies (nAbs or bAbs) in NHP serum samples (n=16 individual animals). All individuals included in the AAV dosing groups were negatively tested in the neutralizing antibody assay (nAb-assay). Animal AJ562 showed a low reactivity against IgGs (below cut-off <1:4) in the binding antibody assay which was considered not to affect AAV transduction (bAb-assay) after dosing. Animal AJ574 (PBS control group) was positively tested in both nAb and bAb (1<64). See Tables 3, 4 and 5).

TABLE 4
Summary of pre-existing immunity in 16 cynomolgus monkeys

| | Nab titer | | | BAb titer | |
|---|---|---|---|---|---|
| Animal ID | AAV2-WT | BI-15.1 | BI-15.2 | BI-15.1 | BI-15.2 |
| AJ180 | — | — | — | — | — |
| AJ760 | — | — | — | — | — |
| AJ672 | 1:8 | — | — | 1:16 | 1:8 |
| AJ658 | 1:8 | — | — | 1:8 | 1:4 |
| AZ013 | — | — | | — | — |
| AZ135 | 1:8 | 1:2 | 1:2 | 1:8 | 1:8 |
| AZ058 | 1:2 | — | — | — | — |
| AY613 | — | — | | — | — |
| AY563 | 1:16 | 1:2 | 1:1 | 1:8 | 1:8 |
| AJ249 | 1:1 | — | — | — | — |
| AY895 | — | — | — | — | — |
| AY867 | 1:8 | — | — | 1:4 | 1:4 |
| AZ018 | 1:16 | 1:2 | 1:2 | 1:8 | 1:4 |
| AY684 | — | — | — | 1:8 | 1:4 |
| AJ574 | 1:256 | 1:8 | 1:8 | 1:64 | 1:64 |
| AJ562 | — | — | — | 1:2 | 1:2 |

TABLE 5
Summary of pre-existing Immunity in 16 cynomolgus monkeys

| AAV-variant | positive NAb >2 | positive BAD ≥2 |
|---|---|---|
| BI-15.2 | 4 out of 16 | 9 out of 16 |
| BI-15.1 | 4 out of 16 | 9 out of 16 |
| AAV2-WT | 8 out of 16 | n.d. |

BI-15.1 and BI-15.2 vectors were intravenously injected with 1×10¹³ vg/kg BW (Table 3) and vector genomes distribution profile was examined three weeks postinjection. For BI-15.1, relevant vector copy numbers were detected in the cardiac ventricle, the atrium, the liver and spleen (FIG. 7A). In contrast, BI-15.2 vector DNA was predominantly detected in the liver and spleen. This is shown by a distinct cardiac versus liver ratio in the BI-15.1 or BI-15.2 treated animal groups (FIG. 7A, 8A). Therefore, it can be concluded that BI-15.1 has an improved car-10 diac homing profile compared to BI-15.2. However, for both BI-15.1 as well as BI-15.2, no capsid mediated homing was observed in various other tissues as shown by vector DNA quantification (FIG. 7A, 8A). As shown, BI-15.1 and BI-15.2 vector mediated gene expression correlated with vector distribution. We show a strong myocardic eGFP expression in the atrium as well as in the cardiac ventricle, while only moderate transduction was observed in the liver. Based on mRNA analysis, BI-15.2 also showed some transduction of the skeletal muscle. Various other tissues showed no expression based on the eGFP RNA level (FIG. 7B, 8B). As expected from the previous results in rats, BI-15.1 mediated expression outperformed BI-15.2, presumably duo to the higher activity of the CAG-promoter. In addition, we corroborated the results from mRNA based gene expression profiles by immunohistochemical staining of tissue sections. We observed a strong but focal eGFP signal of myocardial cells at the base of the heart as well as the cardiac ventricle, confirming our previous results. In other off-target tissues, positive staining for eGFP was mainly limited to single hepatocytes, neurons and germinal centers of the spleen (FIG. 7C, FIG. 8C). Organs without eGFP positive cells were: lung, eye, uterus, spinal cord, pancreas, skeletal muscle, and kidney. For quantification of the gene expression pattern in sagittal whole heart sections, we performed area quantification for eGFP positive cells. For BI-15.1 this results in up to ˜23% percent positive-stained cells in the atrium and up to ˜15% positive cells in the cardiac ventricle (FIG. 7D). In addition, ELISA quantification of the eGFP in tissue lysates confirmed these results (FIG. 7E). The lower transduction of cardiomyocytes by BI-15.2 compared to BI-15.1 was due to the lower vector performance resulting from reduced cardiac homing combined with lower expression activity mediated by the CMV-promoter (FIG. 8D, 8E).

Mainly two features compliment the unique targeting properties of BI-15.1 and BI-15.2 to target cardiac tissue in NHP and to express genes specifically in the heart. Peptide mediated receptor targeting to cardiac tissue/cells via, but not limited to, receptors, receptor sub-classes or cellular carbohydrates presented on the target tissue and de-targeting of the liver. Both features are dependent on the individual design of the peptide, their unique stuffer sequences as well as the specific insertion-site (R588) that was used for BI-15.1 and BI-15.2. In addition, their unique properties of BI-15.1 and BI-15.2 to target endothelial cells in mice might indicate that endothelial structures in mice could be a surrogate model for targeting cardiomyocytes in rats and NHPs and hiPSC-derived human cardiomyocytes.

### 2.6 Vectors Expressing Cardiac Repair Factors in

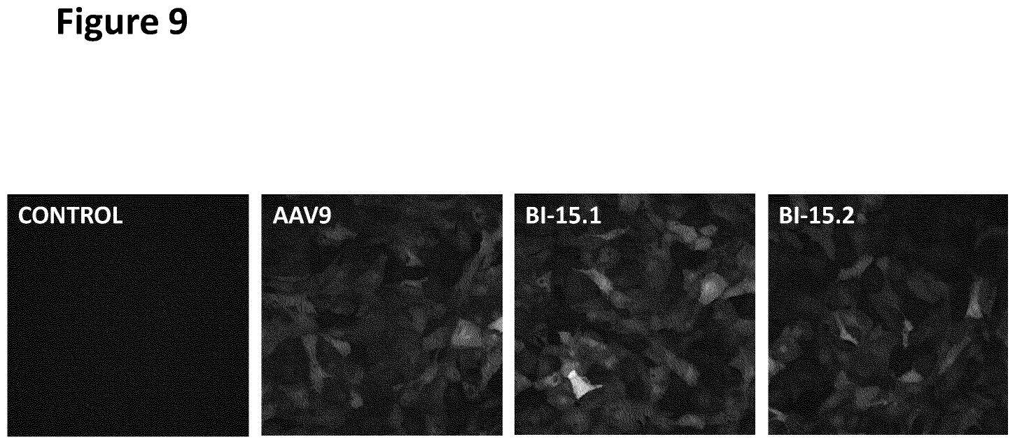

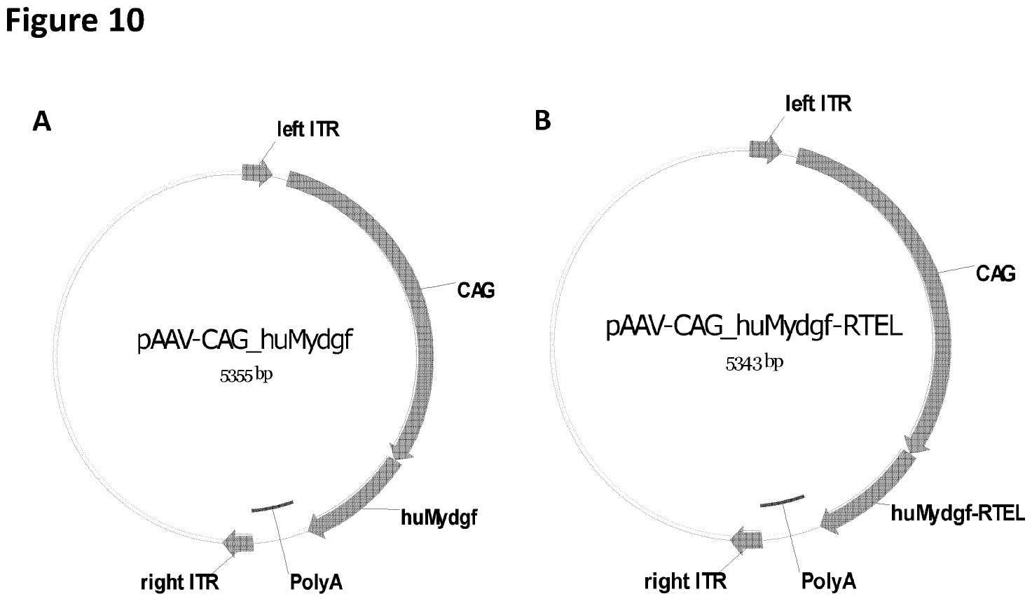

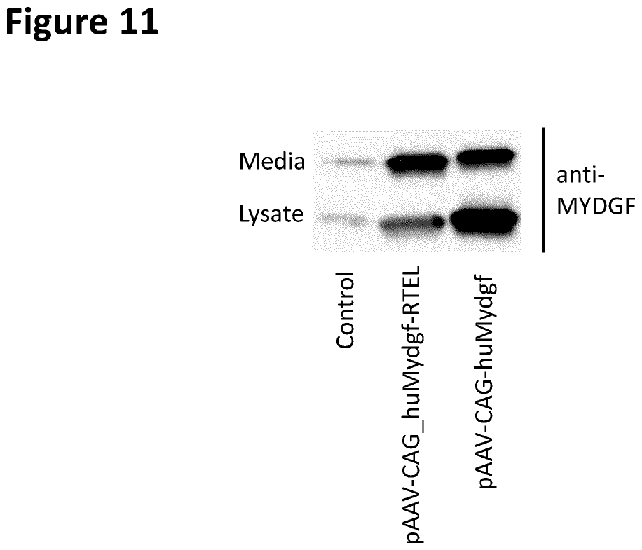

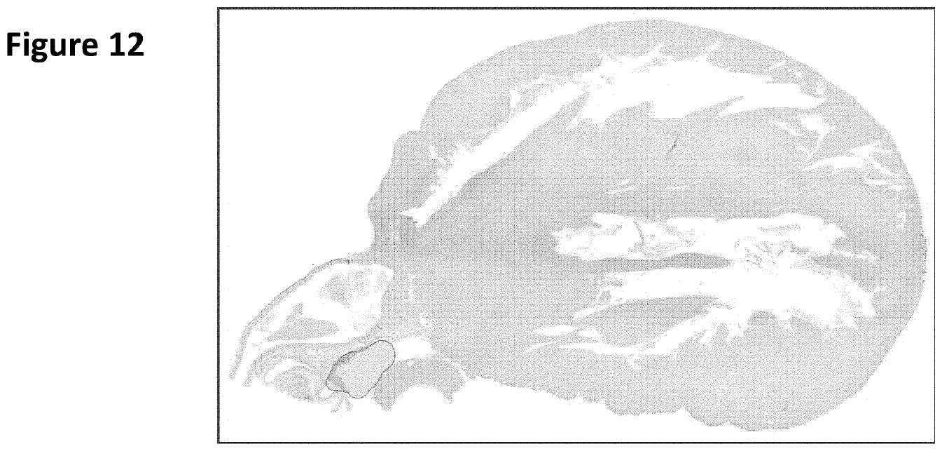

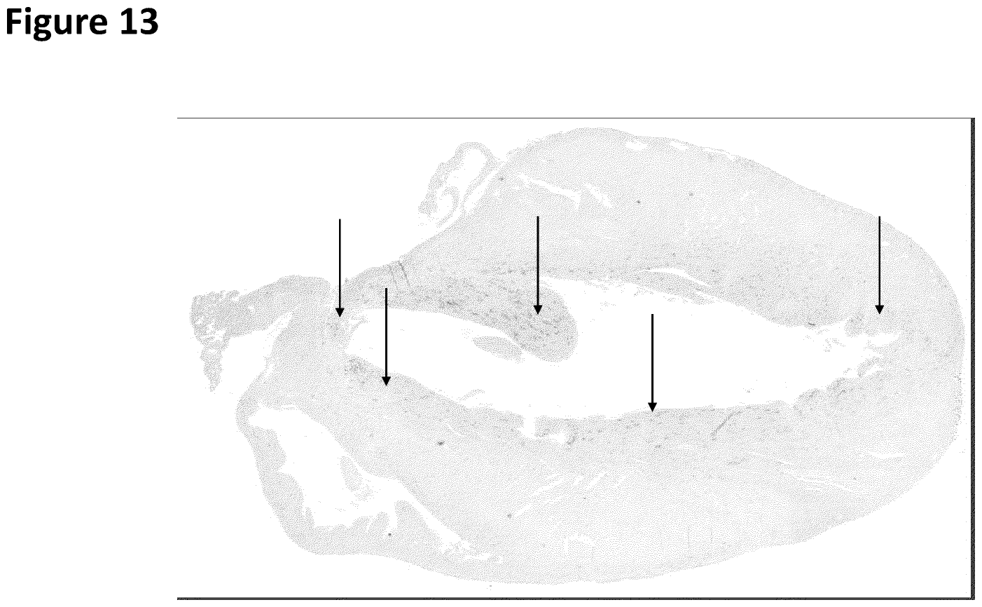

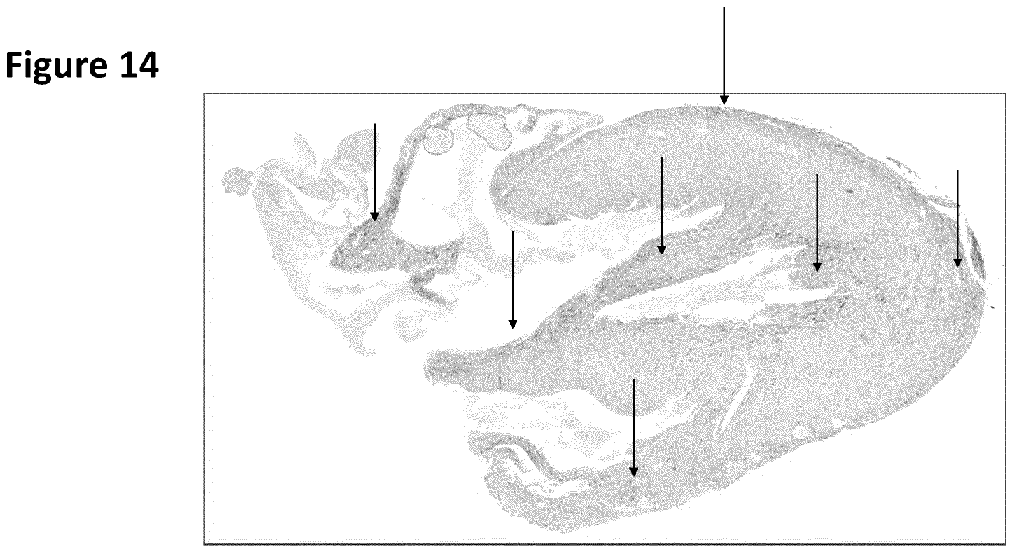

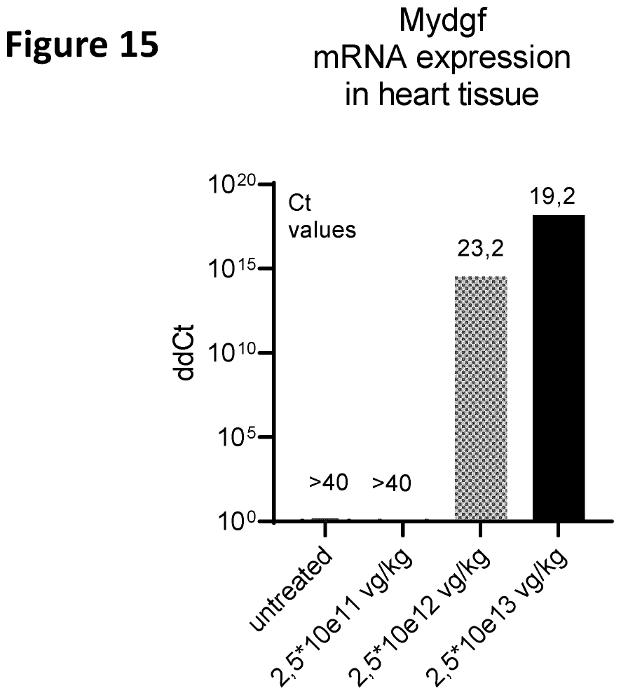

Cardiomyopathies are the leading cause for heart failure (Writing Group et al, 2016). Gene therapy expressing cardiac repair factors, such as Mydgf (Korf-Klingebiel et al, 2015, 2016; Korf-Klingebiel et al, 2021) delivered specifically to cardiac tissue using BI-15.1 may ameliorate cardiac hypotrophy and cardiac fibrosis to restore cardiac function. To explore the clinical potential to deliver genes to cardiac tissues of patients, BI-15.1 and BI-15.2 were tested on human cardiomyocytes. Therefore, hiPSCs-derived human cardiomyocytes were transduced with of BI-15.1, BI-15.2 and AAV9 as a control. 48 hours later eGFP expression was analyzed by fluorescence imaging. All vectors transduced hiPSC-derived human cardiomyocytes at comparable efficacy (FIG. 9). To address if Mydgf (Bortnov et al, 2018; Bortnov et al, 2019) can be expressed from pAAV expression plasmids, we transfected plasmid DNA encoding Mydgf under control of the CAG promoter (FIG. 10). 48 hours after transfection immunoblot analysis showed that Mydgf expressed from pAAV constructs is mainly retained in the cellular fraction (lysate) while the mutant version in which the terminal four amino acids RTEL were deleted (Mydgf-RTEL) gets extensively secreted into the media (FIG. 11). These data show that AAV expressed Mydgf or Mydgf-RTEL can be delivered by BI-15.1 either locally or as secreted factors. To further explore the potential to deliver cardiac repair genes and their expression to cardiac tissue Sprague Dawley rats were intravenously injected with 2.5×10¹¹ vg/kg, 2.5×10¹² vg/kg or 2.5×10¹³ vg/kg of 15.1, transferring Mydgf sequence under the control of the CAG promoter. For analysis of transduction and expression of Mydgf 21 days after injection of 15.1 heart tissue was analyzed for Mydgf protein levels in whole heart slices with immunohistochemistry (IHC, FIGS. 12, 13 and 14) and Mydgf mRNA expression in heart tissue (FIG. 15). For 2.5×10¹² vg/kg and 2.5×10¹³ vg/kg expression of Mydgf could be confirmed by IHC in whole heart slices as well as mRNA levels in 30 heart tissue. Here an AAV 15.1 dose dependent increase in signal, both on mRNA levels as well as staining for Mydgf in tissue was seen. Also, analysis of whole heart slices showed the wide distribution of Mydgf over the total area of the heart for the doses 2.5×10¹² vg/kg and 2.5×10¹³ vg/kg. These data show that 15.1 can be used to induce expression of a cardiac repair factors in the heart in vivo in a species with high relevance as a model organism for human cardiac diseases and for testing therapeutic approaches for cardiac disease (Patten and Hall-Porter, 2009; Riehle et al., 2019)

To show that Mydgf-RTEL variant also has activity and can be used as a therapeutic cargo for the 15.1 vector both variants of Mydgf were applied in a mouse heart ischemia reperfusion model. Mice received a coronary artery ligation followed by protein treatment with recombinant Mydgf and recombinant Mydgf-RTE variant. Proteins were given as initial bolus at the time of reperfusion followed by a 7 day constant exposure. 28 days post operation treatment with both Mydgf variants showed positive effect on left ventricle remodeling and systolic dysfunction (FIG. 16 A, B) in comparison to operated and placebo treated mice as well as positive effects on angiogenesis (FIG. 17A) and did reduce infarct size (FIG. 17 B). These data indicate the activity of the Mydgf-RTEL variant, hence support the use of this variant as therapeutic cargo for 15.1 to treat cardiomyopathies.

### LIST OF REFERENCES

Asokan A, Conway J C, Phillips J L, Li C, Hegge J, Sinnott R, Yadav S, DiPrimio N, Nam H J, Agbandje-McKenna M et al (2010) Reengineering a receptor footprint of adeno-associated virus enables selective and systemic gene transfer to muscle. Nat Biotechnol 28: 79-82
Bass-Stringer S, Bernardo B C, May C N, Thomas C J, Weeks K L, McMullen J R. Adeno-Associated Virus Gene Therapy: Translational Progress and Future Prospects in the Treatment of Heart Failure. Heart Lung Circ. 2018 November; 27(11):1285-1300. doi: 10.1016/j.hlc.2018.03.005. Epub 2018 Mar. 17. PMID: 29703647.)
Bortnov V, Annis D S, Fogerty F J, Barretto K T, Turton K B, Mosher D F (2018) Myeloid-derived growth factor is a resident endoplasmic reticulum protein. J Biol Chem 293: 13166-13175
Bortnov V, Tonelli M, Lee W, Lin Z, Annis D S, Demerdash O N, Bateman A, Mitchell J C, Ge Y, Markley J L et al (2019) Solution structure of human myeloid-derived growth factor suggests a conserved function in the endoplasmic reticulum. Nat Commun 10: 5612
Burridge P W, Matsa E, Shukla P, Lin Z C, Churko J M, Ebert A D, Lan F, Diecke S, Huber B, Mordwinkin N M et al (2014) Chemically defined generation of human cardiomyocytes. Nat Methods 11: 855-860
Capitani Mirco, Sallese Michele, The KDEL receptor: New functions for an old protein, FEBS Letters, Volume 583, Issue 23, 2009, Pages 3863-3871, ISSN 0014-5793, https://doi.org/10.1016/j.febslet.2009.10.053.
Chamberlain K, Riyad J M, Weber T (2017) Cardiac gene therapy with adeno-associated virus-based vectors. Curr Opin Cardiol
Chemaly E R, Hajjar R J, Lipskaia L (2013) Molecular targets of current and prospective heart failure therapies. Heart 99: 992-1003
Colella P, Ronzitti G, Mingozzi F (2018) Emerging Issues in AAV-Mediated In Vivo Gene Therapy. Mol Ther Methods Clin Dev 8: 87-104
Drawnel F M, Boccardo S, Prummer M, Delobel F, Graff A, Weber M, Gerard R, Badi L, Kam-Thong T, Bu L et al (2014) Disease modeling and phenotypic drug screening for diabetic cardiomyopathy using human induced pluripotent stem cells. Cell Rep 9: 810-821
Ebenhoch R., Akhdar A., Reboll M. R., Korf-Klingebiel M., Gupta P., Armstrong J., et al. Crystal structure and receptor-interacting residues of MYDGF—a protein mediating ischemic tissue repair. Nat Commun. 2019 November; 10 (1): 1-10.
England J, Granados-Riveron J, Polo-Parada L, Kuriakose D, Moore C, Brook J D, Rutland C S, Setchfield K, Gell C, Ghosh T K et al (2017) Tropomyosin 1: Multiple roles in the developing heart and in the formation of congenital heart defects. J Mol Cell Cardiol 106: 1-13
Foinquinos A, Batkai S, Genschel C, Viereck J, Rump S, Gyongyosi M, Traxler D, Riesenhuber M, Spannbauer A, Lukovic D et al (2020) Preclinical development of a miR132 inhibitor for heart failure treatment. Nat Commun 11: 633
Greenberg B, Butler J, Felker G M, Ponikowski P, Voors A A, Desai A S, Barnard D, Bouchard A, Jaski B, Lyon A R et al (2016) Calcium upregulation by percutaneous administration of gene therapy in patients with cardiac disease (CUPID 2): a randomised, multinational, double-blind, placebo-controlled, phase 2b trial. Lancet 387: 1178-1186
Greenberg B, Butler J, Felker G M, Ponikowski P, Voors A A, Desai A S, Barnard D, Bouchard A, Jaski B, Lyon A R, Pogoda J M, Rudy J J, Zsebo K M. Calcium upregulation by percutaneous administration of gene therapy in patients with cardiac disease (CUPID 2): a randomised, multinational, double-blind, placebo-controlled, phase 2b trial. Lancet. 2016 Mar. 19; 387(10024):1178-86. doi: 10.1016/S0140-6736(16)00082-9. Epub 2016 Jan. 21. PMID: 26803443.
Hordeaux J, Wang Q, Katz N, Buza E L, Bell P, Wilson J M (2018) The Neurotropic Properties of AAV-PHP.B Are Limited to C57BL/6J Mice. Mol Ther 26: 664-668
Irene Gil-Farina, Raffaele Fronza, Christine Kaeppel, Esperanza Lopez-Franco, Valerie Ferreira, Delia D'Avola, Alberto Benito, Jesus Prieto, Harald Petry, Gloria Gonzalez-Aseguinolaza, Manfred Schmidt, Recombinant AAV Integration Is Not Associated With Hepatic Genotoxicity in Nonhuman Primates and Patients, Molecular Therapy, Volume 24, Issue 6, 2016, Pages 1100-1105, ISSN 1525-0016, https://doi.org/10.1038/mt.2016.52.
Jessup M, Greenberg B, Mancini D, Cappola T, Pauly D F, Jaski B, Yaroshinsky A, Zsebo K M, Dittrich H, Hajjar R J et al (2011) Calcium Upregulation by Percutaneous Administration of Gene Therapy in Cardiac Disease (CUPID): a phase 2 trial of intracoronary gene therapy of sarcoplasmic reticulum Ca2+-ATPase in patients with advanced heart failure. Circulation 124: 304-313
Korbelin J, Dogbevia G, Michelfelder S, Ridder D A, Hunger A, Wenzel J, Seismann H, Lampe M, Bannach J, Pasparakis M et al (2016a) A brain microvasculature endothelial cell-specific viral vector with the potential to treat neurovascular and neurological diseases. EMBO Mol Med 8: 609-625
Korbelin J, Sieber T, Michelfelder S, Lunding L, Spies E, Hunger A, Alawi M, Rapti K, Indenbirken D, Muller O J et al (2016b) Pulmonary Targeting of Adeno-associated Viral Vectors by Next-generation Sequencing-guided Screening of Random Capsid Displayed Peptide Libraries. Mol Ther 24: 1050-1061
Korf-Klingebiel M, Reboll M R, Klede S, Brod T, Pich A, Polten F, Napp L C, Bauersachs J, Ganser A, Brinkmann E et al (2016) Corrigendum: Myeloid-derived growth factor (C19orf10) mediates cardiac repair following myocardial infarction. Nat Med 22: 446
Korf-Klingebiel M., Reboll M. R., Klede S., Brod T., Pich A., Polten F., et al. Myeloid-derived growth factor (C19orf10) mediates cardiac repair following myocardial infarction. Nat Med. 2015 February; 21 (2): 140-149.
Korf-Klingebiel M, Reboll M R, Polten F, Weber N, Jäckle F, Wu X, Kallikourdis M, Kunderfranco P, Condorelli G, Giannitsis E, Kustikova O S, Schambach A, Pich A, Widder J D, Bauersachs J, Heuvel J, Kraft T, Wang Y, Wollert K C (2021), Myeloid-Derived Growth Factor Protects Against Pressure Overload-Induced Heart Failure by Preserving Sarco/Endoplasmic Reticulum Ca2+-ATPase Expression in Cardiomyocytes, Circulation, 144:1227-1240
Lyon A R, Babalis D, Morley-Smith A C, Hedger M, Suarez Barrientos A, Foldes G, Couch L S, Chowdhury R A, Tzortzis K N, Peters N S, Rog-Zielinska E A, Yang H Y, Welch S, Bowles C T, Rahman Haley S, Bell A R, Rice A, Sasikaran T, Johnson N A, Falaschetti E, Parameshwar J, Lewis C, Tsui S, Simon A, Pepper J, Rudy J J, Zsebo K M, Macleod K T, Terracciano C M, Hajjar R J, Banner N, Harding S E. Investigation of the safety and feasibility of AAV1/SERCA2a gene transfer in patients with chronic heart failure supported with a left ventricular assist device—the SERCA-LVAD TRIAL. Gene Ther. 2020 December; 27 (12): 579-590. doi: 10.1038/s41434-020-0171-7. Epub 2020 Jul. 15. PMID: 32669717; PMCID: PMC7744277.
Morrison M, Klein C, Clemann N, Collier D A, Hardy J, Heisserer B, Cader M Z, Graf M, Kaye J (2015) StemBANCC: Governing Access to Material and Data in a Large Stem Cell Research Consortium. Stem Cell Rev Rep 11: 681-687
Pang J K S, Phua Q H, Soh B S (2019) Applications of miRNAs in cardiac development, disease progression and regeneration. Stem Cell Res Ther 10: 336
Patten R D and Hall-Porter M R (2009), Small Animal Models of Heart Failure, Development of Novel Therapies, Past and Present, Circulation: Heart Failure, 2:138-144
Powell S K, Rivera-Soto R, Gray S J (2015) Viral expression cassette elements to enhance transgene target specificity and expression in gene therapy. Discov Med 19: 49-57
Qiao C, Yuan Z, Li J, He B, Zheng H, Mayer C, Li J, Xiao X (2011) Liver-specific microRNA-122 target sequences incorporated in AAV vectors efficiently inhibits transgene expression in the liver. Gene Ther 18: 403-410
Riehle C and Bauersachs J (2019), Small animal models of heart failure, Cardiovascular Research, Volume 115, Issue 13, 1838-1849
Sakuma T, Barry M A, Ikeda Y; Lentiviral vectors: basic to translational, Biochem J, 443 (3): 603-618
Strobel B, Zuckschwerdt K, Zimmermann G, Mayer C, Eytner R, Rechtsteiner P, Kreuz S, Lamla T (2019) Standardized, Scalable, and Timely Flexible Adeno-Associated Virus Vector Production Using Frozen High-Density HEK-293 Cell Stocks and CELLdiscs. Hum Gene Ther Methods 30: 23-33
Tarantal A F, Lee C C I, Martinez M L, Asokan A, Samulski R J (2017) Systemic and Persistent Muscle Gene Expression in Rhesus Monkeys with a Liver De-Targeted Adeno-Associated Virus Vector. Hum Gene Ther 28: 385-391
Tilemann L, Ishikawa K, Weber T, Hajjar R J (2012) Gene therapy for heart failure. Circ Res 110: 777-793
Ucar A, Gupta S K, Fiedler J, Erikci E, Kardasinski M, Batkai S, Dangwal S, Kumarswamy R, Bang C, Holzmann A et al (2012) The miRNA-212/132 family regulates both cardiac hypertrophy and cardiomyocyte autophagy. Nat Commun 3: 1078
Wilson J M, Flotte T R (2020) Moving Forward After Two Deaths in a Gene Therapy Trial of Myotubular Myopathy. Hum Gene Ther 31: 695-696
Writing Group M, Mozaffarian D, Benjamin E J, Go A S, Arnett D K, Blaha M J, Cushman M, Das S R, de Ferranti S, Despres J P et al (2016) Heart Disease and Stroke Statistics-2016 Update: A Report From the American Heart Association. Circulation 133: e38-360
Xiao X, Li J, Samulski R J (1998) Production of high-titer recombinant adeno-associated virus vectors in the absence of helper adenovirus. J Virol 72:2224-2232
Xu T, Zhou Q, Che L, Das S, Wang L, Jiang J, Li G, Xu J, Yao J, Wang H et al (2016) Circulating miR-21, miR-378, and miR-940 increase in response to an acute exhaustive exercise in chronic heart failure patients. Oncotarget 7: 12414-12425
Yang L, Jiang J, Drouin L M, Agbandje-McKenna M, Chen C, Qiao C, Pu D, Hu X, Wang D Z, Li J et al (2009) A myocardium tropic adeno-associated virus (AAV) evolved by DNA shuffling and in vivo selection. Proc Natl Acad Sci USA 106: 3946-3951
Yamada K P, Tharakan S, Ishikawa K. Consideration of clinical translation of cardiac AAV gene therapy. Cell Gene Ther Insights. 2020; 6(5):609-615. doi:10.18609/cgti.2020.073
Ying Y, Muller O J, Goehringer C, Leuchs B, Trepel M, Katus H A, Kleinschmidt J A (2010) Heart-targeted adeno-associated viral vectors selected by in vivo biopanning of a random viral display peptide library. Gene Ther 17: 980-990
Zsebo K, Yaroshinsky A, Rudy J J, Wagner K, Greenberg B, Jessup M, Hajjar R J (2014) Long-term effects of AAV1/SERCA2a gene transfer in patients with severe heart failure: analysis of recurrent cardiovascular events and mortality. Circ Res 114: 101-108
Zsebo K, Yaroshinsky A, Rudy J J, Wagner K, Greenberg B, Jessup M, Hajjar R J. Long-term effects of AAV1/SERCA2a gene transfer in patients with severe heart failure: analysis of recurrent cardiovascular events and mortality. Circ Res. 2014 Jan. 3; 114(1):101-8. doi: 10.1161/CIRCRESAHA.113.302421. Epub 2013 Sep. 24. PMID: 24065463.

1.-2. (canceled)

3. A method of treating or preventing a heart disease in a primate, wherein said method comprises the transduction of primate cardiomyocytes with a capsid protein, said capsid protein comprising
(a) the amino acid sequence of SEQ ID NO:1;
(b) the amino acid sequence of SEQ ID NO:2 or 3; or
(c) a variant of (a) or (b) which differs from the sequence of SEQ ID NO:1, SEQ ID NO: 2 or SEQ ID NO:3 by the modification of one amino acid.

4. (canceled)

5. The method of claim 3, wherein said capsid protein:
(a) has a length of 300 to 800 amino acids, or
(b) is a capsid protein of a virus, preferably belonging to the Parvoviridae family, and preferably a capsid protein of an adeno-associated virus (AAV).

6. (canceled)

7. The method of claim 3, wherein said capsid is a capsid protein of an adeno-associated virus (AAV), wherein said AAV is selected from the group consisting of AAV serotype 2, 4, 6, 8 and 9, and wherein said AAV is preferably serotype 2.

8. The method of claim 7, wherein said capsid protein is a VP1 protein of an AAV serotype 2.

9. The method of claim 3, wherein said amino acid sequence of SEQ ID NO:1, SEQ ID NO:2 or SEQ ID NO: 3 or said variant thereof is inserted in the region of amino acids 550-600 of the capsid protein.

10. The method of claim 3, wherein said capsid protein comprises:
(a) the amino acid sequence of SEQ ID NO: 7 or SEQ ID NO: 8;
(b) an amino acid sequence having at least 90% identity to the amino acid sequence of SEQ ID NO: 7 or SEQ ID NO: 8; or
(c) a fragment of one of the amino acid sequences defined in (a) or (b).

11. The method of claim 3, wherein said capsid protein comprises:
(a) the amino acid sequence of SEQ ID NO:24;
(b) the amino acid sequence of SEQ ID NO:25;
(c) the amino acid sequence of SEQ ID NO:26;
(d) the amino acid sequence of SEQ ID NO:27; or
(e) a variant of (a), (b), (c) or (d) which differs from the sequence of SEQ ID NO:24, SEQ ID NO:25, SEQ ID NO:26 or SEQ ID NO:27, respectively, by the modification of one amino acid.

12. (canceled)

13. The method of claim 3, said method comprising administering to the primate a nucleic acid encoding said capsid protein.

14. The method of claim 13, said method comprising administering to the primate a plasmid which comprises said nucleic acid, preferably a viral vector in which the plasmid is used which encodes the capsid protein.

15. Recombinant viral vector, wherein the vector comprises a capsid and a transgene packaged therein, wherein the capsid comprises at least one capsid protein comprising
(a) the amino acid sequence of SEQ ID NO:1;
(b) the amino acid sequence of SEQ ID NO:2;
(c) the amino acid sequence of SEQ ID NO:3;
(d) a variant of (a), (b) or (c) which differs from the sequence of SEQ ID NO:1, SEQ ID NO: 2, or SEQ ID NO:3 by the modification of one amino acid;
(e) the amino acid sequence of SEQ ID NO:24;
(f) the amino acid sequence of SEQ ID NO:25;
(g) the amino acid sequence of SEQ ID NO:26;
(h) the amino acid sequence of SEQ ID NO:27;
(i) a variant of (e), (f), (g) or (h) which differs from the sequence of SEQ ID NO:24, SEQ ID NO:25, SEQ ID NO:26, SEQ ID NO:27 by the modification of one amino acid,
(j) the amino acid sequence of SEQ ID NO:7 or SEQ ID NO:8; or
(k) an amino acid sequence having at least 90% identity to the amino acid sequence of SEQ ID NO:7 or SEQ ID NO:8;
for use
(A) in a method of treating or preventing a heart disease in a primate, wherein said method of treating or preventing a heart disease comprises the transduction of primate cardiomyocytes; or
(B) in a method of treating or preventing cardiomyopathy in a primate; or
(C) in a method of treating or preventing heart failure or chronic heart failure in a primate.

16.-20. (canceled)

21. Recombinant viral vector according to claim 15, wherein said vector is a recombinant AAV vector, wherein said AAV is selected from the group consisting of AAV serotype 2, 4, 6, 8 and 9, and preferably AAV serotype 2.

22. (canceled)

23. Recombinant viral vector according to claim 15, wherein the transgene is in the form of an ssDNA or a dsDNA.

24. (canceled)

25. Recombinant viral vector according to claim 15, wherein the transgene encodes:
a cardiac repair factor huMydgf;
a calcium regulator selected from the group consisting of SERCA2a, SUMO1, and S100A1;
a pro-angiogenic factor VEGF; or
a microRNA (miRNA) involved in the regulation of the MAPK pathway, the MYOD pathway, the FOXO3 pathway, or the ERK-MAPK pathway.

26.-29. (canceled)

30. Recombinant viral vector according to claim 25, wherein said microRNA is selected from the group consisting of miR378, miR669a, miR-21 miR212, and miR132.

31.-34. (canceled)

35. Recombinant viral vector according to claim 25, wherein said huMydgf comprises
(a) the amino acid sequence of SEQ ID NO: 18, 20, 33 or 34;
(b) an amino acid sequence having at least 90% identity to the amino acid sequence of SEQ ID NO: 18, 20, 33 or 34; or
(c) a fragment of one of the amino acid sequences defined in (a) or (b).

36. (canceled)

37. Recombinant viral vector according to claim 25, wherein the huMydgf lacks a functional Golgi/endoplasmatic reticulum retention signal, preferably a huMydgf that comprises the sequence of SEQ ID NO:20 or SEQ ID NO:34.

38.-39. (canceled)

40. Recombinant viral vector according to claim 25, wherein the human calcium regulator SERCA2a comprises
(a) an amino acid sequence of any of SEQ ID NOs: 28-32;
(b) an amino acid sequence having at least 90% identity to one of the amino acid sequences of SEQ ID NOs: 28-32; or
(c) a fragment of one of the amino acid sequences defined in (a) or (b).

41.-42. (canceled)

43. Pharmaceutical composition comprising a recombinant viral vector of claim 15, wherein said composition is preferably formulated for intravenous administration.

44.-45. (canceled)

46. A composition comprising isolated heart tissue cells of a rat or a primate, preferably isolated cardiomyocytes, wherein said isolated heart tissue cells have been transduced with a recombinant viral vector of claim 15.

47. The method of claim 3, wherein said heart disease is:
a cardiomyopathy selected from the group consisting of hypertrophic cardiomyopathy (HCM), dilated cardiomyopathy (DCM), arrythmogenic right ventricular cardiomyopathy (ARVC), restrictive cardiomyopathy (RCM) left ventricular noncompaction cardiomyopathy (LVNC), inherited cardiomyopathy, cardiomyopathy caused by spontaneous mutations, and acquired cardiomyopathy, preferably ischemic cardiomyopathy caused by atherosclerotic or other coronary artery diseases, cardiomyopathy caused by infection or intoxication of the myocardium; or
a condition selected from the group consisting of angina pectoris, cardiac fibrosis and cardiac hypertrophy; or
heart failure with preserved ejection fraction (HFpEF), heart failure with reduced ejection fraction (HFrEF), or heart failure with mid-range ejection fraction (HFmrEF).

48. The method of claim 14, wherein said viral vector comprises said capsid and a transgene packaged therein, wherein the transgene is a gene that supplants a defective gene in the primate to be treated, wherein said gene encodes a protein selected from the group of beta-myosin heavy chain (MYH7), myosin binding protein C (MYBPC3), troponin I (TNNI3), troponin T (TNNT2), tropomyosin alpha-1 chain (TPM1), and myosin light chain (MYL3).


SEQUENCE LISTING (37 sequences)

SEQ ID NO: 1 - protein, 7 aa, Artificial Sequence
  Peptide 1
NRGTEWD

SEQ ID NO: 2 - protein, 5 aa, Artificial Sequence
  Peptide 2
GHGYF

SEQ ID NO: 3 - protein, 7 aa, Artificial Sequence
  Peptide 3
ESGHGYF

SEQ ID NO: 4 - protein, 735 aa, Adeno-associated virus - 2 [sequence omitted]

SEQ ID NO: 5 - protein, 598 aa, Adeno-associated virus - 2
  VP2 protein
MAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 6 - protein, 533 aa, Adeno-associated virus - 2
  VP3 protein
MATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 7 - protein, 744 aa, Adeno-associated virus - 2
  VP1 modified with peptide 1
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGQRGNRGTEWDAQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 8 - protein, 744 aa, Adeno-associated virus - 2
  VP1 modified with peptide 3
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGQRGESGHGYFAQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 9 - DNA, 8359 bp, Artificial Sequence
  cap/rep plasmid modified with insert coding for peptide 1
catcatcaataatataccttattttggattgaagccaatatgataatgagggggtggagtttgtgacgtggcgcggggcgtgggaacggggcgggtgacgtagtagctctagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggcaccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggccagagaggcaatcgggggactgagtgggatgcccaggcggccaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgcgtatttctttcttatctagtttccatgctctagaggtcctgtattagaggtcacgtgagtgttttgcgacattttgcgacaccatgtggtcacgctgggtatttaagcccgagtgagcacgcagggtctccattttgaagcgggaggtttgaacgcgcagccaccacggcggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcatcgctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaattcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagaattcc

SEQ ID NO: 10 - DNA, 8359 bp, Artificial Sequence
  cap/rep plasmid modified with insert coding for peptide 3
catcatcaataatataccttattttggattgaagccaatatgataatgagggggtggagtttgtgacgtggcgcggggcgtgggaacggggcgggtgacgtagtagctctagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggcaccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggccagagaggcgagtctggtcatggttattttgcccaggcggccaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgcgtatttctttcttatctagtttccatgctctagaggtcctgtattagaggtcacgtgagtgttttgcgacattttgcgacaccatgtggtcacgctgggtatttaagcccgagtgagcacgcagggtctccattttgaagcgggaggtttgaacgcgcagccaccacggcggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcatcgctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaattcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagaattcc

SEQ ID NO: 11 - DNA, 23 bp, Artificial Sequence
  Primer forward
cgtcaatgggtggagtatttacg

SEQ ID NO: 12 - DNA, 23 bp, Artificial Sequence
  Primer reverse
aggtcatgtactgggcataatgc

SEQ ID NO: 13 - DNA, 34 bp, Artificial Sequence
  Probe
agtacatcaagtgtatcatatgccaagtacgccc

SEQ ID NO: 14 - DNA, 1617 bp, Artificial Sequence
  CAG-Promoter
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaacatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagacaattgtactaaccttcttctctttcctctcctgacaggttggtgtacagtagcttcca

SEQ ID NO: 15 - DNA, 221 bp, Simian virus 40
  polyadenylation sequence
cagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggt

SEQ ID NO: 16 - DNA, 140 bp, Adeno-associated virus - 2
  ITR
cctgcaggcagctgcgcgctcgctcgctcactgaggccgcccgggcaccgcccgggcgtcgggcgacctttggtcgcccggcctcagtgagcgagcgagcgcgcagagagggagtggccaactccatcactaggggttcc

SEQ ID NO: 17 - DNA, 140 bp, Adeno-associated virus - 2
  ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcag

SEQ ID NO: 18 - protein, 173 aa, Homo sapiens
  huMydgf protein sequence with signal peptide
MAAPSGGWNGVGASLWAALLLGAVALRPAEAVSEPTTVAFDVRPGGVVHSFSHNVGPGDKYTCMFTYASQGGTNEQWQMSLGTSEDHQHFTCTIWRPQGKSYLYFTQFKAEVRGAEIEYAMAYSKAAFERESDVPLKTEEFEVTKTAVAHRPGAFKAELSKLVIVAKASRTEL

SEQ ID NO: 19 - DNA, 522 bp, Homo sapiens
  huMydgf gene sequence
atggcggcgcccagcggagggtggaacggcgtcggcgcgagcttgtgggccgcgctgctcctaggggccgtggcgctgaggccggcggaggcggtgtccgagcccacgacggtggcgtttgacgtgcggcccggcggcgtcgtgcattccttctcccataacgtgggcccgggggacaaatatacgtgtatgttcacttacgcctctcaaggagggaccaatgagcaatggcagatgagtctggggaccagcgaagaccaccagcacttcacctgcaccatctggaggccccaggggaagtcctatctgtacttcacacagttcaaggcagaggtgcggggcgctgagattgagtacgctatggcctactctaaagccgcatttgaaagggaaagtgatgtccctctgaaaactgaggaatttgaagtgaccaaaacagcagtggctcacaggcccggggcattcaaagctgagctgtccaagctggtgattgtggccaaggcatcgcgcactgagctgtga

SEQ ID NO: 20 - protein, 169 aa, Artificial Sequence
  mutant huMydgf protein sequence with signal peptide
MAAPSGGWNGVGASLWAALLLGAVALRPAEAVSEPTTVAFDVRPGGVVHSFSHNVGPGDKYTCMFTYASQGGTNEQWQMSLGTSEDHQHFTCTIWRPQGKSYLYFTQFKAEVRGAEIEYAMAYSKAAFERESDVPLKTEEFEVTKTAVAHRPGAFKAELSKLVIVAKAS

SEQ ID NO: 21 - DNA, 510 bp, Artificial Sequence
  mutant huMydgf gene sequence
atggcggcgcccagcggagggtggaacggcgtcggcgcgagcttgtgggccgcgctgctcctaggggccgtggcgctgaggccggcggaggcggtgtccgagcccacgacggtggcgtttgacgtgcggcccggcggcgtcgtgcattccttctcccataacgtgggcccgggggacaaatatacgtgtatgttcacttacgcctctcaaggagggaccaatgagcaatggcagatgagtctggggaccagcgaagaccaccagcacttcacctgcaccatctggaggccccaggggaagtcctatctgtacttcacacagttcaaggcagaggtgcggggcgctgagattgagtacgctatggcctactctaaagccgcatttgaaagggaaagtgatgtccctctgaaaactgaggaatttgaagtgaccaaaacagcagtggctcacaggcccggggcattcaaagctgagctgtccaagctggtgattgtggccaaggcatcgtga

SEQ ID NO: 22 - DNA, 2757 bp, Artificial Sequence
  pAAV plasmids harbouring huMydgf
cctgcaggcagctgcgcgctcgctcgctcactgaggccgcccgggcaccgcccgggcgtcgggcgacctttggtcgcccggcctcagtgagcgagcgagcgcgcagagagggagtggccaactccatcactaggggttcctgcggcccgataggctggactctgcaccataacacacaatcaacaggggagtgagctggatcgagctagagtccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaacatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagacaattgtactaaccttcttctctttcctctcctgacaggttggtgtacagtagcttccaccatggcggcgcccagcggagggtggaacggcgtcggcgcgagcttgtgggccgcgctgctcctaggggccgtggcgctgaggccggcggaggcggtgtccgagcccacgacggtggcgtttgacgtgcggcccggcggcgtcgtgcattccttctcccataacgtgggcccgggggacaaatatacgtgtatgttcacttacgcctctcaaggagggaccaatgagcaatggcagatgagtctggggaccagcgaagaccaccagcacttcacctgcaccatctggaggccccaggggaagtcctatctgtacttcacacagttcaaggcagaggtgcggggcgctgagattgagtacgctatggcctactctaaagccgcatttgaaagggaaagtgatgtccctctgaaaactgaggaatttgaagtgaccaaaacagcagtggctcacaggcccggggcattcaaagctgagctgtccaagctggtgattgtggccaaggcatcgcgcactgagctgtgaaagcttgcctcgaggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcag

SEQ ID NO: 23 - DNA, 2745 bp, Artificial Sequence
  pAAV plasmids harbouring huMydgf-RTEL
cctgcaggcagctgcgcgctcgctcgctcactgaggccgcccgggcaccgcccgggcgtcgggcgacctttggtcgcccggcctcagtgagcgagcgagcgcgcagagagggagtggccaactccatcactaggggttcctgcggcccgataggctggactctgcaccataacacacaatcaacaggggagtgagctggatcgagctagagtccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaacatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagacaattgtactaaccttcttctctttcctctcctgacaggttggtgtacagtagcttccaccatggcggcgcccagcggagggtggaacggcgtcggcgcgagcttgtgggccgcgctgctcctaggggccgtggcgctgaggccggcggaggcggtgtccgagcccacgacggtggcgtttgacgtgcggcccggcggcgtcgtgcattccttctcccataacgtgggcccgggggacaaatatacgtgtatgttcacttacgcctctcaaggagggaccaatgagcaatggcagatgagtctggggaccagcgaagaccaccagcacttcacctgcaccatctggaggccccaggggaagtcctatctgtacttcacacagttcaaggcagaggtgcggggcgctgagattgagtacgctatggcctactctaaagccgcatttgaaagggaaagtgatgtccctctgaaaactgaggaatttgaagtgaccaaaacagcagtggctcacaggcccggggcattcaaagctgagctgtccaagctggtgattgtggccaaggcatcgtgaaagcttgcctcgaggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcag

SEQ ID NO: 24 - protein, 9 aa, Artificial Sequence
  Peptide 1 with stuffer
GNRGTEWDA

SEQ ID NO: 25 - protein, 9 aa, Artificial Sequence
  Peptide 3 with stuffer
GESGHGYFA

SEQ ID NO: 26 - protein, 11 aa, Artificial Sequence
  Peptide 1 with stuffer
QRGNRGTEWDA

SEQ ID NO: 27 - protein, 11 aa, Artificial Sequence
  Peptide 3 with stuffer
QRGESGHGYFA

SEQ ID NO: 28 - protein, 1042 aa, Homo sapiens
  AT2A2 - Sarcoplasmic/endoplasmic reticulum calcium ATPase 2 - Isoform 1
MENAHTKTVEEVLGHFGVNESTGLSLEQVKKLKERWGSNELPAEEGKTLLELVIEQFEDLLVRILLLAACISFVLAWFEEGEETITAFVEPFVILLILVANAIVGVWQERNAENAIEALKEYEPEMGKVYRQDRKSVQRIKAKDIVPGDIVEIAVGDKVPADIRLTSIKSTTLRVDQSILTGESVSVIKHTDPVPDPRAVNQDKKNMLFSGTNIAAGKAMGVVVATGVNTEIGKIRDEMVATEQERTPLQQKLDEFGEQLSKVISLICIAVWIINIGHFNDPVHGGSWIRGAIYYFKIAVALAVAAIPEGLPAVITTCLALGTRRMAKKNAIVRSLPSVETLGCTSVICSDKTGTLTTNQMSVCRMFILDRVEGDTCSLNEFTITGSTYAPIGEVHKDDKPVNCHQYDGLVELATICALCNDSALDYNEAKGVYEKVGEATETALTCLVEKMNVFDTELKGLSKIERANACNSVIKQLMKKEFTLEFSRDRKSMSVYCTPNKPSRTSMSKMFVKGAPEGVIDRCTHIRVGSTKVPMTSGVKQKIMSVIREWGSGSDTLRCLALATHDNPLRREEMHLEDSANFIKYETNLTFVGCVGMLDPPRIEVASSVKLCRQAGIRVIMITGDNKGTAVAICRRIGIFGQDEDVTSKAFTGREFDELNPSAQRDACLNARCFARVEPSHKSKIVEFLQSFDEITAMTGDGVNDAPALKKAEIGIAMGSGTAVAKTASEMVLADDNFSTIVAAVEEGRAIYNNMKQFIRYLISSNVGEVVCIFLTAALGFPEALIPVQLLWVNLVTDGLPATALGFNPPDLDIMNKPPRNPKEPLISGWLFFRYLAIGCYVGAATVGAAAWWFIAADGGPRVSFYQLSHFLQCKEDNPDFEGVDCAIFESPYPMTMALSVLVTIEMCNALNSLSENQSLLRMPPWENIWLVGSICLSMSLHFLILYVEPLPLIFQITPLNVTQWLMVLKISLPVILMDETLKFVARNYLEPGKECVQPATKSCSFSACTDGISWPFVLLIMPLVIWVYSTDTNFSDMFWS

SEQ ID NO: 29 - protein, 997 aa, Homo sapiens
  AT2A2 - Sarcoplasmic/endoplasmic reticulum calcium ATPase 2 - Isoform 2
MENAHTKTVEEVLGHFGVNESTGLSLEQVKKLKERWGSNELPAEEGKTLLELVIEQFEDLLVRILLLAACISFVLAWFEEGEETITAFVEPFVILLILVANAIVGVWQERNAENAIEALKEYEPEMGKVYRQDRKSVQRIKAKDIVPGDIVEIAVGDKVPADIRLTSIKSTTLRVDQSILTGESVSVIKHTDPVPDPRAVNQDKKNMLFSGTNIAAGKAMGVVVATGVNTEIGKIRDEMVATEQERTPLQQKLDEFGEQLSKVISLICIAVWIINIGHFNDPVHGGSWIRGAIYYFKIAVALAVAAIPEGLPAVITTCLALGTRRMAKKNAIVRSLPSVETLGCTSVICSDKTGTLTTNQMSVCRMFILDRVEGDTCSLNEFTITGSTYAPIGEVHKDDKPVNCHQYDGLVELATICALCNDSALDYNEAKGVYEKVGEATETALTCLVEKMNVFDTELKGLSKIERANACNSVIKQLMKKEFTLEFSRDRKSMSVYCTPNKPSRTSMSKMFVKGAPEGVIDRCTHIRVGSTKVPMTSGVKQKIMSVIREWGSGSDTLRCLALATHDNPLRREEMHLEDSANFIKYETNLTFVGCVGMLDPPRIEVASSVKLCRQAGIRVIMITGDNKGTAVAICRRIGIFGQDEDVTSKAFTGREFDELNPSAQRDACLNARCFARVEPSHKSKIVEFLQSFDEITAMTGDGVNDAPALKKAEIGIAMGSGTAVAKTASEMVLADDNFSTIVAAVEEGRAIYNNMKQFIRYLISSNVGEVVCIFLTAALGFPEALIPVQLLWVNLVTDGLPATALGFNPPDLDIMNKPPRNPKEPLISGWLFFRYLAIGCYVGAATVGAAAWWFIAADGGPRVSFYQLSHFLQCKEDNPDFEGVDCAIFESPYPMTMALSVLVTIEMCNALNSLSENQSLLRMPPWENIWLVGSICLSMSLHFLILYVEPLPLIFQITPLNVTQWLMVLKISLPVILMDETLKFVARNYLEPAILE

SEQ ID NO: 30 - protein, 999 aa, Homo sapiens
  AT2A2 - Sarcoplasmic/endoplasmic reticulum calcium ATPase 2 - Isoform 3
MENAHTKTVEEVLGHFGVNESTGLSLEQVKKLKERWGSNELPAEEGKTLLELVIEQFEDLLVRILLLAACISFVLAWFEEGEETITAFVEPFVILLILVANAIVGVWQERNAENAIEALKEYEPEMGKVYRQDRKSVQRIKAKDIVPGDIVEIAVGDKVPADIRLTSIKSTTLRVDQSILTGESVSVIKHTDPVPDPRAVNQDKKNMLFSGTNIAAGKAMGVVVATGVNTEIGKIRDEMVATEQERTPLQQKLDEFGEQLSKVISLICIAVWIINIGHFNDPVHGGSWIRGAIYYFKIAVALAVAAIPEGLPAVITTCLALGTRRMAKKNAIVRSLPSVETLGCTSVICSDKTGTLTTNQMSVCRMFILDRVEGDTCSLNEFTITGSTYAPIGEVHKDDKPVNCHQYDGLVELATICALCNDSALDYNEAKGVYEKVGEATETALTCLVEKMNVFDTELKGLSKIERANACNSVIKQLMKKEFTLEFSRDRKSMSVYCTPNKPSRTSMSKMFVKGAPEGVIDRCTHIRVGSTKVPMTSGVKQKIMSVIREWGSGSDTLRCLALATHDNPLRREEMHLEDSANFIKYETNLTFVGCVGMLDPPRIEVASSVKLCRQAGIRVIMITGDNKGTAVAICRRIGIFGQDEDVTSKAFTGREFDELNPSAQRDACLNARCFARVEPSHKSKIVEFLQSFDEITAMTGDGVNDAPALKKAEIGIAMGSGTAVAKTASEMVLADDNFSTIVAAVEEGRAIYNNMKQFIRYLISSNVGEVVCIFLTAALGFPEALIPVQLLWVNLVTDGLPATALGFNPPDLDIMNKPPRNPKEPLISGWLFFRYLAIGCYVGAATVGAAAWWFIAADGGPRVSFYQLSHFLQCKEDNPDFEGVDCAIFESPYPMTMALSVLVTIEMCNALNSLSENQSLLRMPPWENIWLVGSICLSMSLHFLILYVEPLPLIFQITPLNVTQWLMVLKISLPVILMDETLKFVARNYLEPVLSSEL

SEQ ID NO: 31 - protein, 1015 aa, Homo sapiens
  AT2A2 - Sarcoplasmic/endoplasmic reticulum calcium ATPase 2 - Isoform 4
MENAHTKTVEEVLGHFGVNESTGLSLEQVKKLKERWGSNELPAEEGKTLLELVIEQFEDLLVRILLLAACISFVLAWFEEGEETITAFVEPFVILLILVANAIVGVWQERNAENAIEALKEYEPEMGKVYRQDRKSVQRIKAKDIVPGDIVEIAGESVSVIKHTDPVPDPRAVNQDKKNMLFSGTNIAAGKAMGVVVATGVNTEIGKIRDEMVATEQERTPLQQKLDEFGEQLSKVISLICIAVWIINIGHFNDPVHGGSWIRGAIYYFKIAVALAVAAIPEGLPAVITTCLALGTRRMAKKNAIVRSLPSVETLGCTSVICSDKTGTLTTNQMSVCRMFILDRVEGDTCSLNEFTITGSTYAPIGEVHKDDKPVNCHQYDGLVELATICALCNDSALDYNEAKGVYEKVGEATETALTCLVEKMNVFDTELKGLSKIERANACNSVIKQLMKKEFTLEFSRDRKSMSVYCTPNKPSRTSMSKMFVKGAPEGVIDRCTHIRVGSTKVPMTSGVKQKIMSVIREWGSGSDTLRCLALATHDNPLRREEMHLEDSANFIKYETNLTFVGCVGMLDPPRIEVASSVKLCRQAGIRVIMITGDNKGTAVAICRRIGIFGQDEDVTSKAFTGREFDELNPSAQRDACLNARCFARVEPSHKSKIVEFLQSFDEITAMTGDGVNDAPALKKAEIGIAMGSGTAVAKTASEMVLADDNFSTIVAAVEEGRAIYNNMKQFIRYLISSNVGEVVCIFLTAALGFPEALIPVQLLWVNLVTDGLPATALGFNPPDLDIMNKPPRNPKEPLISGWLFFRYLAIGCYVGAATVGAAAWWFIAADGGPRVSFYQLSHFLQCKEDNPDFEGVDCAIFESPYPMTMALSVLVTIEMCNALNSLSENQSLLRMPPWENIWLVGSICLSMSLHFLILYVEPLPLIFQITPLNVTQWLMVLKISLPVILMDETLKFVARNYLEPGKECVQPATKSCSFSACTDGISWPFVLLIMPLVIWVYSTDTNFSDMFWS

SEQ ID NO: 32 - protein, 997 aa, Homo sapiens
  AT2A2 - Sarcoplasmic/endoplasmic reticulum calcium ATPase 2 - Isoform 5
MENAHTKTVEEVLGHFGVNESTGLSLEQVKKLKERWGSNELPAEEGKTLLELVIEQFEDLLVRILLLAACISFVLAWFEEGEETITAFVEPFVILLILVANAIVGVWQERNAENAIEALKEYEPEMGKVYRQDRKSVQRIKAKDIVPGDIVEIAVGDKVPADIRLTSIKSTTLRVDQSILTGESVSVIKHTDPVPDPRAVNQDKKNMLFSGTNIAAGKAMGVVVATGVNTEIGKIRDEMVATEQERTPLQQKLDEFGEQLSKVISLICIAVWIINIGHFNDPVHGGSWIRGAIYYFKIAVALAVAAIPEGLPAVITTCLALGTRRMAKKNAIVRSLPSVETLGCTSVICSDKTGTLTTNQMSVCRMFILDRVEGDTCSLNEFTITGSTYAPIGEVHKDDKPVNCHQYDGLVELATICALCNDSALDYNEAKGVYEKVGEATETALTCLVEKMNVFDTELKGLSKIERANACNSVIKQLMKKEFTLEFSRDRKSMSVYCTPNKPSRTSMSKMFVKGAPEGVIDRCTHIRVGSTKVPMTSGVKQKIMSVIREWGSGSDTLRCLALATHDNPLRREEMHLEDSANFIKYETNLTFVGCVGMLDPPRIEVASSVKLCRQAGIRVIMITGDNKGTAVAICRRIGIFGQDEDVTSKAFTGREFDELNPSAQRDACLNARCFARVEPSHKSKIVEFLQSFDEITAMTGDGVNDAPALKKAEIGIAMGSGTAVAKTASEMVLADDNFSTIVAAVEEGRAIYNNMKQFIRYLISSNVGEVVCIFLTAALGFPEALIPVQLLWVNLVTDGLPATALGFNPPDLDIMNKPPRNPKEPLISGWLFFRYLAIGCYVGAATVGAAAWWFIAADGGPRVSFYQLSHFLQCKEDNPDFEGVDCAIFESPYPMTMALSVLVTIEMCNALNSLSENQSLLRMPPWENIWLVGSICLSMSLHFLILYVEPLPLIFQITPLNVTQWLMVLKISLPVILMDETLKFVARNYLEPDIIK

SEQ ID NO: 33 - protein, 142 aa, Homo sapiens
  human Mydgf without signal peptide (mature protein)
VSEPTTVAFDVRPGGVVHSFSHNVGPGDKYTCMFTYASQGGTNEQWQMSLGTSEDHQHFTCTIWRPQGKSYLYFTQFKAEVRGAEIEYAMAYSKAAFERESDVPLKTEEFEVTKTAVAHRPGAFKAELSKLVIVAKASRTEL

SEQ ID NO: 34 - protein, 138 aa, Homo sapiens
  human Mydgf without signal peptide without golgi retention signal (-RTEL)
VSEPTTVAFDVRPGGVVHSFSHNVGPGDKYTCMFTYASQGGTNEQWQMSLGTSEDHQHFTCTIWRPQGKSYLYFTQFKAEVRGAEIEYAMAYSKAAFERESDVPLKTEEFEVTKTAVAHRPGAFKAELSKLVIVAKAS

SEQ ID NO: 35 - protein, 143 aa, Homo sapiens
  Mydgf (WT) protein activity test probe compound protein sequence
MVSEPTTVAFDVRPGGVVHSFSHNVGPGDKYTCMFTYASQGGTNEQWQMSLGTSEDHQHFTCTIWRPQGKSYLYFTQFKAEVRGAEIEYAMAYSKAAFERESDVPLKTEEFEVTKTAVAHRPGAFKAELSKLVIVAKASRTEL

SEQ ID NO: 36 - protein, 173 aa, Homo sapiens
  Mydgf-RTEL protein activity test probe compound pre-protein sequence with signal peptide
MGWSLILLFLVAVATRVLSHHHHHHAGSENLYFQGVSEPTTVAFDVRPGGVVHSFSHNVGPGDKYTCMFTYASQGGTNEQWQMSLGTSEDHQHFTCTIWRPQGKSYLYFTQFKAEVRGAEIEYAMAYSKAAFERESDVPLKTEEFEVTKTAVAHRPGAFKAELSKLVIVAKAS

SEQ ID NO: 37 - protein, 154 aa, Homo sapiens
  Mydgf-RTEL protein activity test probe compound protein sequence without signal peptide
HHHHHHAGSENLYFQGVSEPTTVAFDVRPGGVVHSFSHNVGPGDKYTCMFTYASQGGTNEQWQMSLGTSEDHQHFTCTIWRPQGKSYLYFTQFKAEVRGAEIEYAMAYSKAAFERESDVPLKTEEFEVTKTAVAHRPGAFKAELSKLVIVAKAS